(19) United States
(12) Patent Application Publication
(10) Pub. No.: US 2021/0268028 A1
(43) Pub. Date: Sep. 2, 2021

(54) CHIMERIC ANTIGEN RECEPTORS (CAR)-EXPRESSING CELLS AND COMBINATION TREATMENT FOR IMMUNOTHERAPY OF PATIENTS WITH RELAPSE REFRACTORY ADVERSE GENETIC RISK AML

(71) Applicant: CELLECTIS, Paris (FR)

(72) Inventors: Stéphane André DEPIL, Lyon (FR); Ghulam MUFTI, Greater London (GB); David SOURDIVE, Levallois-Perret (FR)

(21) Appl. No.: 17/256,434

(22) PCT Filed: Jun. 14, 2019
(86) PCT No.: PCT/EP2019/065761

§ 371 Date: Dec. 28, 2020

(30) Foreign Application Priority Data
Jul. 2, 2018 (EP) PCT/EP2018/067857
Nov. 28, 2018 (EP) PCT/EP2018/082913

(51) Int. Cl. A61K 35/17; C07K 14/705; C07K 14/725; C12N 5/10; C12N 5/0783; A61K 31/7076; A61K 31/675

(52) U.S. Cl. CPC A61K 35/17; C07K 14/70596; C07K 14/7051; C12N 2510/00; C12N 5/0636; A61K 31/7076; A61K 31/675; C12N 5/10

(57) ABSTRACT

The present invention relates to compositions comprising engineered allogenic immune cells endowed with Chimeric Antigen Receptors (CAR), in particular a CAR specific for CD123 and CLL1 for treating AML patients with adverse genetic risk.

### FIELD OF THE INVENTION

The present invention relates to the field of cell immunotherapy and more particularly to an engineered immune cell expressing an anti-tumor antigen specific chimeric antigen receptor (such as an anti-CD123 CAR) and composition comprising the same, for the treatment of patients suffering AML with adverse genetic risk and/or with less than 20% over total cells blast in bone marrow. Thus, the present invention encompasses a composition comprising, optionally, a debulking treatment for reducing blasts in the bone marrow to less than 20%, a lymphodepleting treatment, and at least one dose of engineered immune cells expressing specific chimeric antigen receptors, which advantageously originate from the same donor as the cells for the eventual bone marrow transplant. These compositions comprising engineered immune cells and expressing an anti-tumor antigen specific chimeric antigen receptor are particularly efficient in patients with remaining bone marrow blast content, preferably less than 20%, obtained after 1 or 2 courses of standard intensive induction chemotherapy as a debulking treatment and a lymphodepletion. The methods of the present invention resulted in no or mild CRS higher than grade 1 and optimal condition for bone marrow transplantation. The invention thereby provides with compositions comprising CAR immune cells highly efficient and significantly increasing the survival of patients with adverse genetic risk AML.

### BACKGROUND OF THE INVENTION

Acute Myeloid Leukaemia (AML) is a devastating clonal hematopoietic stem cell neoplasm characterized by uncontrolled proliferation and accumulation of leukemic blasts in the bone marrow, peripheral blood, and occasionally in other tissues. These cells disrupt normal haematopoiesis and rapidly cause bone marrow failure and death (Estey, 2014). AML is the most common type of acute leukaemia in adults with an annual incidence rate of 4.2/100000, and a 5 years survival of only 26.9% and a median age at diagnosis of 68 in the United States (www.seer.cancer.gov.accessed in 2018).

While outcomes for younger patients have improved somewhat during the last three decades, mostly due to advances in supportive care, the dismal outcomes for older patients have remained essentially unchanged. Unfortunately, the majority of patients with AML experience disease relapses, including those who achieve initial complete remission. Allogeneic hematopoietic stem cell transplantation in second remission offers the only chance for long-term survival, but this option is not available to most relapsed AML patients, either because they do not achieve second remission, or they cannot tolerate the procedure (Estey and Döhner, 2006).

While there are multiple multi-agent chemotherapy salvage regimens for relapsed AML (e.g. MEC (mitoxantrone, etoposide, and cytarabine) and FLAG-IDA (fludarabine, cytarabine, idarubicin, granulocyte colony-stimulating factor), there is no accepted standard of care because none of these regimens is superior to the others and none results in long-term survival (Roboz, 2012; Roboz et al., 2014).

Clinical trials are recommended in the NCCN (National Comprehensive Cancer Network) and other guidelines, and relapsed AML is widely recognized as an urgent unmet medical need. AML patients with complex cytogenetic abnormalities and/or TP53 mutations (i.e. classified into the ELN Adverse genetic risk group (Döhner et al., 2010 and 2017; Röllig et al., 2011) specifically fall into the category of urgent unmet medical need, as these patients have especially dismal outcomes with all existing treatment modalities, including allogeneic transplantation (Middeke et al., 2016; Rücker et al., 2012; Yanada et al., 2016).

Knowledge of disease biology allows determination of factors that confer a particularly bad outcome to AML with adverse genetic risk. These include complex karyotype, monosomal karyotype and molecular abnormalities including FLT3-ITD, DNMT3A and TP53 mutations. Recent research has resulted in an explosion of clinical and genetic data that have significantly advanced knowledge of disease prognostication (Metzeler et al., 2016; Papaemmanuil et al., 2016). Despite these advances in knowledge no real advances in the therapy of AML have occurred in the past thirty years (Döhner et al., 2017). Mutations in TP53 are of particular importance with adverse prognosis being observed even at a low variant allele frequency of >6% (Goel et al., 2016). Current standard of care for patients with adverse genetic risk AML is to progress to haematopoietic stem cell transplantation (HSCT), however despite this attempt at curative therapy, only a maximum of 20% of patients survive in the longer term. Similar data is reported for those with monosomal karyotype with survival after transplantation of at best 10% (Della Porta et al., 2014). Lindsley (Lindsley et al., 2017) has recently detailed the poor prognosis of TP53 mutation in an analysis of 1514 patients receiving HSCT. Responses were poor in this group even at ages of <40 years, and myeloablative conditioning did not improve prognosis. The median survival of patients with TP53 mutation in this cohort was dismal at <10 months. Yoshizato (Yoshizato et al., 2017) confirms these findings and has published that those with TP53 and complex karyotype have a survival of only 4.8 months after transplantation with more than 80% dying within 2 years of HSCT. These dismal figures raise the question as to whether HSCT should be offered given the poor outcomes, with many authors arguing for the development of novel therapies for these groups of patients.

Once relapse occurs re-induction of remission is usually not obtainable with these patients subsequently dying as a result of progressive AML (Forman et al., 2013).

The adverse features identified in the literature with resultant poor outcomes indicate an urgent need to provide alternate therapies for this patient group. This is particularly true for patients who do not achieve remission after the first course of induction chemotherapy. Further attempts to induce remission with traditional chemotherapy are complicated by toxicity associated with chemotherapy and significant infections rendering patients ineligible for further attempts at curative therapy with HSCT. In addition to those who fail to achieve morphological remission, multiple studies now show that the presence of cytogenetic or minimal residual disease prior to transplantation results in inferior survival post HSCT. As an example, Araki (Araki et al., JCO 2015) identified that patients with minimal residual disease had post-transplant relapse rates of >60% and 3-year overall survival of <20%. These results were the same as for those who had active disease. Identifying treatment options that have the ability to achieve remission and deepen or even eliminate the leukaemia clone prior to HSCT would be of significant advantage in terms of optimizing post-transplant outcomes.

The IL3-Receptor (IL3-R) is a heterodimer which contains two chains: alpha and beta. This heterodimer, along with IL-5 and GM-CSF receptors, all share a common beta subunit, with the alpha chain being unique to each of the three similar cytokine receptors. The IL3 Receptor alpha (IL3Rα), also known as CD123, is overexpressed in patients with hematologic malignancies, particularly myeloid leukaemia [Testa et al., (2014) CD 123 is a membrane biomarker and a therapeutic target in hematologic malignancies. Biomarker Research 2:4].

CD123 is constitutively expressed on normal, committed haematopoietic progenitor cells, and is also expressed in a variety of haematological neoplasms, including AML and myelodysplastic syndrome [Munoz et al., (2001) Haematologica 86: 1261-1269]. The majority of AML blasts express surface CD123, irrespective of AML subtype, and CD123 expression is at a higher density than observed in normal CD34⁺ cells. High levels of CD123 expression are found on CD34⁺ CD38⁻ leukaemia stem cells (LSCs), in contrast to minimal or absent expression on HSCs in normal bone marrow [Taussig et al., (2005) Hematopoietic stem cells express multiple myeloid markers: implications for the origin and targeted therapy of acute myeloid leukemia. Blood. 106:4086-4092]. Leukaemia stem cells are resistant to conventional cytotoxic chemotherapy and are believed to be responsible for disease relapse and expression of CD123 on >1% AML LSCs is associated with a poor prognosis [Vergez et al., (2011) High levels of CD34+CD38low/−CD123+ blasts are predictive of an adverse outcome in acute myeloid leukemia: a Groupe Ouest-Est des Leucémies Aiguës et Maladies du Sang (GOELAMS) study. Haematologica 96: 1792-1798).

Among others, CLL1 (C-Type Lectin-Like Molecule-1) appears to be an interesting tumoral antigen target as it is expressed by leukemic blasts at diagnosis from 85-92% of AML patients analysed It is a 75 kDa member of the group V C-type lectin-like receptor family of molecules. Group V molecules have a lectin-like domain that binds to non-sugar ligands. CLL1 is a 265 aminoacid type II transmembrane glycoprotein (Uniprot database: Q5QGZ9 for human protein encoded by gene n° 160364 in “Entrez Gene” database) that contains a 200 AA extracellular domain. CLL1 is also referred to in the literature and databases as MICL, CLEC12 and KLRL1.

Bakker et al. [C-Type Lectin-Like Molecule-1: A Novel Myeloid Cell Surface Marker Associated with Acute Myeloid Leukemia (2004) Cancer Research 64, 8443-8450] have shown that the CLL1 antigen is associated with AML stem cells. Like some other antigens (such as CD33), CLL1 is a cell surface protein that is specifically expressed on most malignant lymphoid stem cells (AML LSC), while not being expressed on normal HSC. Meanwhile, CLL1 was revealed to be a diagnostic marker in AML [Larsen et al, (2012) Recent advances in acute myeloid leukemia stem cell biology. Haematologica. 97:966-974]. Anti-CLL-1 antibodies enable both AML-specific stem-cell detection and possibly antigen-targeting as distinguishing malignant cells from normal stem cells both at diagnosis and in remission [van Rhenen et al., (2007) The novel AML stem cell-associated antigen CLL-1 aids in discrimination between normal and leukemic stem cells. Blood 110(7):2659-66].

Preclinical investigations using antibodies targeting CD123 for the treatment of AML have been described and have demonstrated promising antileukemic activity in murine models (Majeti, 2011). However, to date, Phase I clinical trials targeting CD123 using monoclonal antibodies and immunotoxins have shown good safety profiles, although only modest responses (Frankel et al., 2008; He et al., 2015), suggesting that alternative and more potent therapies targeting CD123 may be required to observe better antileukemic activity.

Chimeric antigen receptors as well as artificial T-cell receptors (TCRs) are designed to convey an MHC-independent target recognition to a T-cell and trigger the killing of cells harbouring this antigen at their surface. Chimeric antigen receptors are synthetic transmembrane constructs composed of an extracellular single-chain variable fragment (scFv) linked to intracellular T-cell signalling domains, usually CD3ζ chain, with one or more co-stimulatory domains, such as CD28, 4-1BB (CD137), or ICOS (CD278). The signalling properties of CARs are determined by the properties of the signalling domains incorporated into their cytoplasmic tails. It is now well established that CARs that combine both activating and co-stimulatory signalling domains create much more robust anti-tumor effects than an activation domain alone. Combination between the CD137 (also known as 4-1BB) and TCR CD3 zeta cytoplasmic signalling domains were shown to support efficient lysis of tumor cells, as well as sustained T-cell proliferation in vitro and memory formation in vivo (Carpenito et al., 2009; Imai et al., 2004).

Highly promising clinical data have been obtained using T-cells expressing chimeric antigen receptors (CARs). Several groups have developed CARs targeting various antigens for the treatment of B-cell malignancies, and demonstrated that T-cells engineered with anti-CD19 CARs show potent and durable anti-tumor activity in B-cell malignancies (Brentjens et al., 2013; Davila et al., 2014a; Kochenderfer et al., 2015; Lee et al., 2015; Maude et al., 2014a; Park et al., 2016). From first to fourth generation, CAR T-cell technology has developed rapidly. To date, the construction of CAR T-cells has primarily focused on the use of autologous cells in which a patient's own T-cells are modified to express a CAR targeting a tumor surface antigen. Various CARs have been designed for lymphomas and solid tumours. The most promising data have been seen in B-cell acute lymphoblastic leukaemia (B-ALL), in which prolonged CRs have been observed (Davila et al., 2014a; Lee et al., 2015; Park et al., 2016). The majority of patients who achieve CR after treatment with CAR T-cells proceed to allogeneic stem cell transplantation because this is the standard of care in second remission or beyond, but prolonged responses have also been seen in patients unable to undergo transplant, and it is possible that with further development, CAR-T therapy could stand “on its own,” without being followed by allogeneic transplant.

Cells endowed with CAR or with recombinant TCR may be isolated from the patient himself (for autologous transfer) or from a healthy donor (for allogenic transfer). In this later case, cells should be engineered at least for inhibiting the expression of the alpha beta T Cell Receptor at the cell surface (UCART cells, for universal CART) and abrogate the TCR-mediated graft versus host disease (GVHD). Thus, in T cells Major histocompatibility Complex (MHC) class I molecules may also be inactivated by gene editing, to reduce Host Versus Graft Disease during immunotherapy, and reduce or delay elimination of engineered cells by host.

Methods for producing genetically engineered immune cells for allogeneic cell immunotherapy purposes by using rare-cutting endonucleases have been described by the applicant in several prior patent applications, in particular WO2013176915. Such engineered immune cells originating from donors and endowed with CAR have been successfully used in patients for treating CD19 positive tumors, referred to as “UCART19” product, have shown therapeutic potential in at least two infants who had refractory leukemia [Leukaemia success heralds wave of gene-editing therapies (2015) Nature 527:146-147].

A distinct pattern of adverse reactions has been observed with CAR T-cells. On-target on-tumor reactions, including cytokine release syndrome (CRS) remain the main threat to patients treated with CAR T-cells.

Cytokine release syndrome (CRS), is an acute inflammatory process characterized by a substantial but transient elevation of serum cytokines. CRS is often observed in patients treated with CAR T cell therapy whether after adoptive transfer of autologous or allogenic cells. The grade of CRS is directly related to the tumor mass that may reach a kilogram of cells in AML patients with adverse cytogenetic risk [Giavridis, T. et al. (2018) CAR T cell-induced cytokine release syndrome is mediated by macrophages and abated by IL-1 blockade, Nature Medicine. 24:731-738]. To reduce the severity of CRS, several treatments have been proposed, including the use of the IL-6R antagonist tocilizumab. However, CRS can be sudden and is difficult to overcome remaining a main cause of toxicity. The present invention provides means for overcoming the problems related to GVHD, CRS, lack of persistence of CART cells and discloses a treatment allowing a surprisingly very efficient bone marrow transplantation.

Thus, the inventors developed a combination for treating AML with adverse genetic risk cancer by using engineered “off-the-shelf” allogeneic therapeutic cells in conjunction with chemotherapies. The therapeutic benefits afforded by this strategy enhanced by the synergistic effects between chemotherapies and immunotherapy, greatly improved engraftment during bone marrow transplant and survival while reducing side effects (CRS and GVHD).

### SUMMARY OF THE INVENTION

The inventors have generated a composition greatly improving survival of patients suffering AML with adverse genetic risk, whereas these patients had received an induction chemotherapy treatment that was only partially effective (up to 20% blasts remaining in bone marrow) thereby impairing chances of a successful remission.

More specifically, they have used a therapeutic composition comprising a dose of allogeneic engineered immune cells expressing an exogenous recombinant TCR or a chimeric antigen receptor (CAR) CAR+_TCRαβ−_T-, specific for a tumoral antigen present on patient's blasts, for the sequential treatment of said patients with adverse genetic risk AML. These compositions have shown to be particularly effective after a first induction chemotherapy, in view of performing a bone marrow transplant in said patients.

In particular, they have designed a reengineered T-cells expressing a CAR with a specificity to CD123 antigen. These anti-CD123 specific CAR are designated CD123 specific CAR or “anti-CD123 CAR”, or more simply “123 CAR”, or “CAR of the invention” indiscriminately. When the engineered cells expressing said CAR targeting CD123 do not originate from the patient himself by from a donor, they are referred as “UCART 123” and are designed for allogeneic use. Such immune cells generally comprise at least an alpha TCR KO gene (resulting in undetectable level of alpha betaTCR at the cell surface) to make them less alloreactive.

These UCART 123 can be given one or twice after an induction chemotherapy or debulking treatment (and preceded by a lymphodepletion) to reduce the risk of graft versus host disease (GVHD) and other side effects due to the activation of the engineered immune cells, thereby improving the rate of HSCT and the overall rate of survival to 50% at one year.

The Inventors have more particularly developed CD123 specific CAR comprising a scFV derived from the antibody Klon43 and identified highly specific and a very selective CARs construction that bind CD123 expressing tumor cells and selectively destroy such cancer cells, while sparing most normal hematopoietic cells.

Primary cells, in general obtained from peripheral blood mononuclear cells (PBMC), are engineered following activation in vitro (e.g. with anti CD3/CD28 coated beads and recombinant IL2), and transduced with polynucleotides expressing these CARs and with reagents, such as specific rare-cutting endonuclease to create non-alloreactive T-cells, (UCART 123) more especially by disruption of a component of TCR (αβ− T-Cell receptors) to prevent Graft versus host reaction. Other attributes deepening the persistence of cells in host, and providing a favorable state for HSCT could also be obtained by gene editing, for instance, to create immune cells resistant to chemotherapy of lymphodepletion drugs. In specific embodiments such T-cells can exhibit an inactivated CD52 gene and/or inactivated beta 2 microglobulin gene (B2M).

The combination treatment of the invention reduces the risk of CRS, allowed preparing patients for bone marrow transplant and favors engraftment and recovery resulting in a higher survival as compared to patients with classical treatments.

The engineered T-cells of the invention are designed to display in-vivo reactivity and selectivity against CD123 positive cells, is used in concomitance with anti-cancer drugs. Further UCART123 are better tolerated than cells having an intact beta-2-microglobulin gene (β2m) when administered twice. In a particular embodiment, the engineered T-cells of the invention remain efficient even after several administrations, making them useful for immunotherapy as a first treatment (induction), as a consolidation treatment, as a treatment in combination with classical anticancer chemotherapy. The polypeptides and polynucleotide sequences encoding the CARs of the present invention are detailed in the present specification.

### BRIEF DESCRIPTION OF THE FIGURES

FIG. 1: Schema of the vector allowing CAR and RQR8 expression (upper panel); CAR and RQR8 structure (lower panel). CAR=chimeric antigen receptor; CD=cluster of differentiation; RQR8=Suicide/Marker/depletion domain; TM=Transmembrane Domaine; anti-CD123 scFv=Single-chain variable fragment recognizing Cluster of Differentiation 123 (also known as IL3RA); 41BB=signaling domain of TNFRSF9; CD3ζ=signaling domain of cluster of differentiation 3 zeta (also known as CD247).

FIG. 2: A) Representations of different allogeneic CART engineering. Left panel un-modified allogeneic T cells, these cells are sensitive to Host T cells (Allo T cells). Middle panel: insertion of the CAR and concomitant inhibition of the TRAC and β2m loci results in TCRαβ⁻, CAR+, MHCclassI⁻ cells that are no longer sensitive to Host T-cells (Allo T cells) but potentially sensitive to Host NK-cell attack. Right Panel: Insertion of NK-inhibitor and CAR with concomitant inactivation of TRAC and β2m results in TCRαβ⁻, CAR⁺, NK inhibitor cells; these cells are insensitive to Host T- and NK-cell attack. B) Designs of the constructs for targeted integration of CAR at TCR alpha (TRAC) locus (left panel) and NK inhibitor at β2m locus (right panel). These designs allow expression of the CAR or the NK inhibitor (such as HLA-E-B2M fusion peptide) under TRAC or B2M promoter, while inactivating TRAC and B2M expression. These constructs are used with site specific endonuclease (such as TALEN®) targeting TRAC and B2M endogenous loci. C) NK inhibitors effect on NK-cell attack on TCRαβ⁻, CAR⁺, NK inhibitor cells. Normalized quantification of MHC negative cells of UCART bearing the tested NK inhibitors after co-culture with NK cells.

FIG. 3: UCART123 possible attributes. 4-1BB=signaling domain of TNFRSF9 (for TNF receptor superfamily member 9); Ab=antibody; CAR=chimeric antigen receptor; CD123=cluster of differentiation 123 (also known as IL3RA for interleukin 3 receptor subunit alpha); CD3ζ=signalling domain of cluster of differentiation 3 zeta (also known as CD247); CD52=cluster of differentiation 52; KO=knockout; RQR8=Marker/depletion polypeptide; scFv=Single-chain variable fragment; TCR=T-Cell Receptor; UCART123=Universal Chimeric Antigen Receptor T-cells targeting CD123.

FIG. 4: Schematic representation of Dose-Escalation (or de scalation) phase to identify the effective dose

FIG. 5: Study design. FC: fludarabine/cyclophosphamide based; FLAG: Fludarabine cytarabine G-CSF; Ida idarubicin; DA: cytarabine+daunorubicin; HSCT: human stem cell transplantation.

FIG. 6: Study Schedule. The DLT (Dose Limiting Toxicity) observation period is 28 days. ¹ Proceed to HSCT after single UCART123 if DLT observed during the DLT observation period, or if Complete Remission (CR) with Minimal Residual Disease (MRD) below 0.01% (by flow cytometry or molecular methods) is achieved. All other patients are considered for a second UCART123 administration following a second Lymphodepletion (LD). ² Proceed to HSCT after second UCART123 infusion, from 2D28 (28 days after second administration). LTFU (Long Term Follow Up)

FIG. 7: UCART123 in vitro activity against primary AML with adverse cytogenetic risk. Estimation of the percentage of dead primary AML cells with adverse cytogenetic risk or not, after co-incubation with UCART123 cells and control cells (TCRαβ KO) at different Effector to Target ratio (E:T).

FIG. 8: In vivo UCART123 activity against primary AML with cytogenetic risk. 8A) Survival curve in PDX-AML2 model treated with control (vehicle), classical treatment (Ara-C); TCRαβ KO T-cells (negative control) or UCART123 cells. 8B) Survival curve in PDX-AML37 model treated with control (vehicle), classical treatment (Ara-C); TCRαβ KO T-cells (negative control) or two different doses of UCART123 cells. The start of the different treatment is indicated by an arrow. T0 correspond to primary AML cells injection.

FIG. 9: Toxicity evaluation of UCART123 product against hematopoietic stem and progenitor cells in vivo

FIG. 10: UCART123 toxicity evaluation using an in vivo competition model. 10A: Quantification in the blood of normal or AML with adverse genetic risk cells (AML) at different time point after injection of PBS, TCRαβ KO T-cells or UCART123 product in humanized NSG mice. 10B) Quantification in the Bone Marrow of normal cells or AML with adverse genetic risk cells (AML) 3 weeks after injection of PBS, TCRαβ KO T-cells or UCART123 product in humanized NSG mice. 10C) Quantification in the Bone Marrow of CD33+(left panel) or CD34+ (right panel) populations 3 weeks after the treatment with PBS, TCRαβ KO T-cells or UCART123 product in humanized NSG mice.

TABLE 1
Sequences of the different CAR components

| Functional | SEQ ID | |
|---|---|---|
| domains | # | Raw amino acid sequence |
| CD8α | SEQ ID | MALPVTALLLPLALLLHAARP |
| signal | NO. 1 | |
| peptide | | |
| Alternative | SEQ ID | METDTLLLWVLLLWVPGSTG |
| signal | NO. 2 | |
| peptide | | |
| FcYRIIIα | SEQ ID | GLAVSTISSFFPPGYQ |
| hinge | NO. 3 | |
| CD8α hinge | SEQ ID | TTTPAPRPPTPAPTIASQPLSLRPE |
| | NO. 4 | ACRPAAGGAVHTRGLDFACD |
| IgG1 hinge | SEQ ID | EPKSPDKTHTCPPCPAPPVAGPSV |
| | NO. 5 | FLFPPKPKDTLMIARTPEVTCVVVD |
| | | VSHEDPEVKFNWYVDGVEVHNAK |
| | | TKPREEQYNSTYRVVSVLTVLHQD |
| | | WLNGKEYKCKVSNKALPAPIEKTIS |
| | | KAKGQPREPQVYTLPPSRDELTKN |
| | | QVSLTCLVKGFYPSDIAVEWESNG |
| | | QPENNYKTTPPVLDSDGSFFLYSK |
| | | LTVDKSRWQQGNVFSCSVMHEAL |
| | | HNHYTQKSLSLSPGK |
| CD8α | SEQ ID | IYIWAPLAGTCGVLLLSLVITLYC |
| transmembrane | NO. 6 | |
| domain | | |
| 41BB | SEQ ID | IISFFLALTSTALLFLLFFLTLRFSVV |
| transmembrane | NO. 7 | |
| domain | | |
| 41BB | SEQ ID | KRGRKKLLYIFKQPFMRPVQTTQE |
| intracellular | NO. 8 | EDGCSCRFPEEEEGGCEL |
| domain | | |
| CD3ζ | SEQ ID | RVKFSRSADAPAYQQGQNQLYNE |
| intracellular | NO. 9 | LNLGRREEYDVLDKRRGRDPEMG |
| domain | | GKPRRKNPQEGLYNELQKDKMAE |
| | | AYSEIGMKGERRRGKGHDGLYQG |
| | | LSTATKDTYDALHMQALPPR |
| Linker | SEQ ID | GGGGSGGGGSGGGGS |
| | NO. 10 | |

TABLE 2
Sequences of different CDRs, VH and VL in the CARs of the invention

| ScFv sequences | SEQ ID # | Raw amino acid sequence |
|---|---|---|
| Klon43 heavy chain variable | SEQ ID NO. 11 | MADYKDIVMTQSHKFMSTSVGDRV |
| region | | NITCKASQNVDSAVAWYQQKPGQS |
| | | PKALIYSASYRYSGVPDRFTGRGSG |
| | | TDFTLTISSVQAEDLAVYYCQQYYS |
| | | TPWTFGGGTKLEIKR |
| Klon43 light chain variable | SEQ ID NO.12 | EVKLVESGGGLVQPGGSLSLSCAA |
| region | | SGFTFTDYYMSWVRQPPGKALEWL |
| | | ALIRSKADGYTTEYSASVKGRFTLS |
| | | RDDSQSILYLQMNALRPEDSATYYC |
| | | ARDAAYYSYYSPEGAMDYWGQGT |
| | | SVTVSS |
| KLON 43 CDR1 | SEQ ID NO. 13 | GFTFTDYY |
| KLON 43 CDR2 | SEQ ID NO. 14 | RSKADGYTT |
| KLON 43 CDR3 | SEQ ID NO. 15 | ARDAAYYSYYSPEGAMDY |
| KLON 43 CDR4 | SEQ ID NO. 16 | QNVDSA |
| KLON 43 CDR5 | SEQ ID NO. 17 | SAS |
| KLON 43 CDR6 | SEQ ID NO. 18 | QQYYSTPWT |
| Humanized scFv Klon43 | SEQ ID NO. 20 | MADYKDIVMTQSPSSVSASVGDRV |
| Variant VL1 | | TITCRASQNVDSAVAWYQQKPGKA |
| | | PKALIYSASYRYSGVPSRFSGRGSG |
| | | TDFTLTISSLQPEDFATYYCQQYYST |
| | | PWTFGQGTKVEIKR |
| Humanized scFv Klon43 | SEQ ID NO. 21 | MADYKDIQMTQSPSSVSASVGDRV |
| Variant VL2 | | TITCRASQNVDSAVAWYQQKPGKA |
| | | PKALIYSASYRYSGVPSRFSGRGSG |
| | | TDFTLTISSLQPEDFATYYCQQYYST |
| | | PWTFGQGTKVEIKR |
| Humanized scFv Klon43 | SEQ ID NO. 22 | MADYKDIQMTQSPSSVSASVGDRV |
| Variant VL3 | | TITCRASQNVDSAVAWYQQKPGKA |
| | | PKALIYSASYRYSGVPSRFSGSGSG |
| | | TDFTLTISSLQPEDFATYYCQQYYST |
| | | PWTFGQGTKVEIKR |
| Humanized scFv Klon43 | SEQ ID NO. 23 | MADYKDIQMTQSPSSVSASVGDRV |
| Variant VL4 | | TITCRASQNVDSAVAWYQQKPGKA |
| | | PKLLIYSASYRYSGVPSRFSGSGSG |
| | | TDFTLTISSLQPEDFATYYCQQYYST |
| | | PWTFGQGTKVEIKR |
| Humanized scFv Klon43 | SEQ ID NO. 24 | MADYKDIQMTQSPSSVSASVGDRV |
| Variant VL5 | | TITCRASQNVDSAVAWYQQKPGKA |
| | | PKLLIYSASYRQSGVPSRFSGSGSG |
| | | TDFTLTISSLQPEDFATYYCQQYYST |
| | | PWTFGQGTKVEIKR |
| Humanized scFv Klon43 | SEQ ID NO. 25 | MADYKDIQMTQSPSSVSASVGDRV |
| Variant VL6 | | TITCRASQNVDSAVAWYQQKPGKA |
| | | PKLLIYSASYGQSGVPSRFSGSGSG |
| | | TDFTLTISSLQPEDFATYYCQQYYST |
| | | PWTFGQGTKVEIKR |
| Humanized scFv Klon43 | SEQ ID NO. 26 | EVKLVESGGGLVQPGRSLRLSCTAS |
| Variant VH1 | | GFTFTDYYMSVWRQAPGKGLEWV |
| | | CLIRSKADGYTTEYSASVKGRFTISR |
| | | DDSKSILYLQMNSLKTEDTAVYYCA |
| | | RDAAYYSYYSPEGAMDYWGQGTLV |
| | | TVSS |
| Humanized scFv Klon43 | SEQ ID NO. 27 | EVQLVESGGGLVQPGRSLRLSCTA |
| Variant VH2 | | SGFTFTDYYMSWVRQAPGKGLEW |
| | | VGLIRSKADGYTTEYSASVKGRFTIS |
| | | RDDSKSILYLQMNSLKTEDTAVYYC |
| | | ARDAAYYSYYSPEGAMDYWGQGTL |
| | | VTVSS |
| Humanized scFv Klon43 | SEQ ID NO. 28 | EVQLVESGGGLVQPGRSLRLSCTA |
| Variant VH3 | | SGFTFTDYYMSWVRQAPGKGLEW |
| | | VGLIRSKADGYTTEYSASVKGRFTIS |
| | | RDDSKSIAYLQMNSLKTEDTAVYYC |
| | | ARDAAYYSYYSPEGAMDYWGQGTL |
| | | VTVSS |
| Humanized scFv Klon43 | SEQ ID NO. 29 | EVQLVESGGGLVQPGRSLRLSCTA |
| Variant VH4 | | SGFTFTDYYMSWVRQAPGKGLEW |
| | | VGFIRSKADGYTTEYSASVKGRFTIS |
| | | RDDSKSIAYLQMNSLKTEDTAVYYC |
| | | ARDAAYYSYYSPEGAMDYWGQGTL |
| | | VTVSS |
| Humanized scFv Klon43 | SEQ ID NO. 30 | EVQLVESGGGLVQPGRSLRLSCTA |
| Variant VH5 | | SGFTFTDYYMSWVRQAPGKGLEW |
| | | VGFIRSKADGYTTEYAASVKGRFTIS |
| | | RDDSKSIAYLQMNSLKTEDTAVYYC |
| | | ARDAAYYSYYSPEGAMDYWGQGTL |
| | | VTVSS |
| Humanized scFv Klon43 | SEQ ID NO. 31 | EVQLVESGGGLVQPGRSLRLSCTA |
| Variant VH6 | | SGFTFTDYYMSWVRQAPGKGLEW |
| | | VGLIRSKADGYTTEYAASVKGRFTIS |
| | | RDDSKSIAYLQMNSLKTEDTAVYYC |
| | | ARDAAYYSYYSPEGAMDYWGQGTL |
| | | VTVSS |
| Humanized scFv Klon43 | SEQ ID NO. 32 | EVQLVESGGGLVQPGRSLRLSCTA |
| Variant VH7 | | SGFTFTDYYMSWVRQAPGKGLEW |
| | | VGFIRSKADGYTTEYAASVKGRFTIS |
| | | RDDSKSIAYLQMNSLKTEDTAVYYC |
| | | TRDAAYYSYYSPEGAMDYWGQGTL |
| | | VTVSS |
| Anti-CLL1 VH | SEQ ID NO. 33 | EVQLQQSGPELVKPGASVKMSCKA |
| | | SGYTFTSYFIHWVKQKPGQGLEWIG |
| | | FINPYNDGSKYNEKFKGKATLTSDK |
| | | SSSTAYMELSSLTSEDSAVYYCTRD |
| | | DGYYGYAMDYWGQGTSVTVSS |
| Anti CLL1 VL | SEQ ID NO. 34 | DIQMTQSPSSLSASLGERVSLTCRA |
| | | TQELSGYLSWLQQKPDGTIKRLIYA |
| | | ASTLDSGVPKRFSGNRSGSDYSLTI |
| | | SSLESEDFADYYCLQYAIYPYTFGG |
| | | GTKLEIKR |

TABLE 3
CAR of structure V-3

| | CAR Structure | | | | | | |
|---|---|---|---|---|---|---|---|
| | signal | | | | | | |
| CAR Designation | peptide | | | CD8α | CD8α | 41BB | CD3ζI |
| V-3 | (optional) | VH | VL | hinge | TM | IC | D |
| Klon43CAR | SEQ ID | SEQ ID | SEQ ID | SEQ ID | SEQ ID | SEQ ID | SEQ ID |
| (SEQ ID NO: 19) | NO: 1 | NO: 11 | NO: 12 | NO: 4 | NO: 6 | NO: 8 | NO: 9 |
| CLL1-CAR | SEQ ID | SEQ ID | SEQ ID | SEQ ID | SEQ ID | SEQ ID | SEQ ID |
| | NO: 1 | NO: 33 | NO: 34 | NO: 4 | NO: 6 | NO: 8 | NO: 9 |

### DETAILED DESCRIPTION OF THE INVENTION

Unless specifically defined herein, all technical and scientific terms used have the same meaning as commonly understood by a skilled artisan in the fields of gene therapy, biochemistry, genetics, molecular biology and medicine.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention is drawn to a method for treating adverse genetic risk AML patient by cell immunotherapy by using composition of engineered immune cells in support of an induction chemotherapy that may initially fail to achieve minimal residual disease (MRD).

This method allows conditioning patients with adverse genetic risk AML in view of obtaining more successful bone marrow transplant.

Adverse genetic risk is defined as per ELN guidelines (below, Döhner et al., 2017) by any of the following genetic signatures:

a. t(6;9)(p23;q34.1); DEK-NUP214; or

b. t(v;11q23.3); KMT2A rearranged; or

c. t(9;22)(q34.1;q11.2); BCR-ABL1; or

d. inv(3)(q21.3q26.2) or t(3;3)(q21.3;q26.2); GATA2, MECOM(EVI1); or

e. -5 or del(5q); -7; -17/abn(17p) or Complex karyotype comprising three or more unrelated chromosome abnormalities in the absence of one of the World Health Organization-designated recurring translocations or inversions, i.e., t(8;21), inv(16) or t(16;16), t(9;11), t(v;11)(v;q23.3), t(6;9), inv(3) or t(3;3); AML with BCR-ABL1; or

f. Monosomal karyotype comprising presence of one single monosomy (excluding loss of X or Y) in association with at least one additional monosomy or structural chromosome abnormality (excluding core-binding factor AML); or

h. Wild-type NPM1 and FLT3-ITD high or

i. Mutated RUNX1 (except if co-occur with favorable-risk AML subtypes) or

j. Mutated ASXL1 (except if co-occur with favorable-risk AML subtypes) or

k. Mutated TP53.

According to some embodiments, the method of the present invention comprises one or several of the following steps of:

i) Induction chemotherapy treatment to reduce blasts in the bone marrow to lower than 20%, although not achieving minimal residual disease (MRD);

ii) lymphodepleting treatment to eliminate, at least partially, patient's own immune cells;

iii) Immunotherapy treatment comprising administering a dose of engineered immune cells expressing a chimeric antigen receptor (CAR) or a recombinant TCR specific for a tumoral antigen marker at the surface membrane of said remaining blasts to achieve MRD;

iv) optionally, administering a second dose of engineered immune cells expressing a chimeric antigen receptor (CAR) until reaching actual MRD;

v) optionally, treating patient with a pre-conditioning regimen prior to bone marrow transplant.

vi) optionally, performing a bone marrow transplant.

Any of these steps can be performed according to the protocols detailed in the examples and clinical trials presented in this specification.

By “induction chemotherapy treatment” is meant an initial systemic treatment using a combination of cytotoxic drugs (i.e. absent any immune cells) to achieve the elimination of maximum cancer cells.

The induction chemotherapy treatments in the present invention can be selected from a combination of an anthracycline (such as daunorubicin [Ex:Cerubidine®], doxorubicin [Ex:Adriamycin® PFS, Adriamycin®] or idarubicin [Ex: Idamycin®]) and cytarabine (also called cytosine arabinoside or ara-C [Ex:Cytosar-U®]);
a combination of an anthracycline, such as daunorubicin [Ex:Cerubidine®], doxorubicin [Ex:Adriamycin® PFS, Adriamycin®] or idarubicin [Idamycin®]), and cytarabine (also called cytosine arabinoside or ara-C [Cytosar-U®]);
Anti CD33 antibody, such as Gemtuzumab ozogamicin (Mylotarg™) for diagnosed AML whose tumors express the CD33 antigen (CD33-positive AML), generally in combination with one of the above drugs;
A protein kinase inhibitor, such as Midostaurin (Rydapt®) especially approved for the treatment of newly diagnosed adult patients with AML that is FLT3 mutation positive, in combination with standard cytarabine and daunorubicin induction and cytarabine consolidation;
A combination of Venetoclax (Venclexta®) with azacitidine or decitabine or low-dose cytarabine for the treatment of newly-diagnosed acute myeloid leukemia (AML) in adults who are age 75 years or older, or who have comorbidities that preclude use of intensive induction chemotherapy. [DiNardo, C. D. et al. (2018) Venetoclax combined with decitabine or azacitidine in treatment-naive, elderly patients with acute myeloid leukemia, Blood; doi: https://doi.org/10.1182/blood-2018-08-868752]
A combination of Glasdegib (Daurismo™) with low-dose cytarabine, especially for the treatment of adult patients who are ≥75 years old or who have comorbidities that preclude use of intensive induction chemotherapy.

The chemotherapy drug cladribine may be added for some patients who can tolerate this compound. Patients with poor heart function, who may not be able to be treated with anthracyclines, may be treated with another chemotherapy drug, such as fludarabine (Fludara) or etoposide.

With respect to adverse genetic risk AML, it has been observed that the above standard front-line treatments do not always achieve complete blast elimination (<0,1% i.e. minimal residual disease), leaving a sub-population of patients, in which 1 to 20% blasts remain after treatment with a very poor diagnosis. This invention aims to solve the situation of these patients, in which MRD cannot be achieved, especially in view of bone marrow transplant.

In general, minimal or, more appropriately, measurable residual disease (MRD) denotes the presence of leukemia cells down to levels of 1:104 to 1:106 white blood cells (WBCs), compared with 1:20 in morphology-based assessments. For the detection of MRD, a comprehensive panel characterized by early marker(s) like CD34 and CD117, myeloid-lineage associated markers, and differentiation antigens like CD2, CD7, CD19, or CD56, are used to track aberrant AML blast cells and analyzed by flow cytometry. MRD determination is preferably performed according to the consensus document from the European LeukemiaNet MRD Working Party [Schuurhuis, G. J. et al. (2018) Minimal/measurable residual disease in AML: a consensus document from the European LeukemiaNet MRD Working Party, Blood 131:1275-1291; doi: https://doi.org/10.1182/blood-2017-09-801498].

According to the invention, a lymphodepleting treatment is generally performed before administering the engineered immune cells to the patients with adverse genetic risk AML.

Such lymphodepleting treatment generally combines fludarabine and cyclophosphamide. Preferably, AML patients with residual cytogenetic or morphological disease with less than 20% blasts are treated with a lymphodepleting regimen comprising fludarabine, preferably between 20 and 40 mg/m2/day and preferably by IV, generally for 3 to 5 days, followed by a higher dose of fludarabine, preferably more than 50 mg combined with cyclophosphamide, preferably more than 0.5 g/m2/day for 2 to 4 days before the immunotherapy starts.

As a preferred embodiment of the present invention, the lymphodepleting treatment can comprise an anti-CD52 antibody, such as alemtuzumab, alone or in combination. The lymphodepletion regimen may for instance combine cyclophosphamide, typically for 1 to 3 days, fludarabine for 1 to 5 days, and alemtuzumab from 1 to 5 days. More specifically, the lymphodepletion regimen can combine between cyclophosphamide 50 and 70 mg/kg/day, fludarabine between 20 and 40 mg/m2/day, and alemtuzumab 0.1 to 0.5 mg/kg/day.

According to the present methods, the above induction chemotherapy treatment and lymphodepletion steps are usually followed by a cell Immunotherapy treatment using engineered immune cells. In general, such engineered immune cells are engineered ex-vivo to modify their immune specificity in order to perform adoptive immunotherapy. They can be genetically modified by using viral vectors and/or transient expression of rare-cutting endonucleases to introduce transgenes or inactivating endogenous genes as further described in the present specification. These techniques have been extensively reviewed in the art, like for instance by Maeder, M. L. and Gersbach, C. A. (2016) Genome-editing Technologies for Gene and Cell Therapy, Molecular Therapy. 24(3): 430-446.

The immune cells may originate from the patients (autologous engineered cells) or from donors (allogenic engineered immune cells). They are generally primary cells obtainable from leukapheresis or derived from iPS cells or cell lines. These Immune cells are generally population of lymphocytes, preferably NK or T-cells. The engineered immune cells of the present invention preferably express recombinant TCR or a chimeric antigen receptor (CAR) specific for an AML tumoral antigen marker. By “recombinant TCR” is meant that an exogenous TCR with a different specificity is introduced or expressed into the cell that partially or completely replace the expression of the endogenous TCR.

By “chimeric antigen receptor” is meant an artificial recombinant receptors that provide both antigen-binding and T-cell-activating functions typically resulting from the fusion of an extracellular domain from the antigen binding regions of both heavy and light chains of a monoclonal antibody, a transmembrane domain, and an endodomain with a signaling domain derived from CD3-ζ. Most CARs further include co-stimulatory signalling endodomains, such as from CD28 or 4-1BB [Dotti, G., Gottschalk, S., Savoldo, B., & Brenner, M. K. (2014). Design and development of therapies using chimeric antigen receptor-expressing T cells. Immunological reviews, 257(1), 107-126]

According to preferred embodiments of the invention said engineered immune cells expressing a chimeric antigen receptor (CAR) or recombinant TCR is specific for a tumoral antigen selected from CD25, CD30, CD37, CD38, CD33, CD47, CD98, CD123, FLT3, CLL-1, CD56, CD117, CD133, CD157, c-kit, CD34, MUC1, CXCR4, VEGF, NKG2D_F, folate receptor beta (FR beta), hepatocyte growth factor (HGF), HLA-A2 and Lewis Y.

According to more preferred embodiments said engineered immune cells express chimeric antigen receptor (CAR) specific for CD123 and/or CLL1 tumoral antigen(s).

The present invention more particularly discloses methods involving specific chimeric antigen receptor (“123 CAR” or “CAR”) expressed at the cell surface of an alpha beta TCR-negative cell in combination with a lymphodepleting treatment for treating patients of AML with adverse cytogenetic risk, in particular of AML with adverse cytogenetic risk in patients with less than 20% blasts in the bone marrow.

The invention encompasses a limited number of CAR specific for tumor antigens expressed by cancer cells in AML, such as CD123,

The CAR of the present invention comprises an extra cellular ligand binding-domain comprising VH and VL from a monoclonal anti-CD123 antibody, such as Klon43, a hinge, a transmembrane domain, a cytoplasmic domain including a CD3 zeta signaling domain and a co-stimulatory domain from 4-1BB, for use in a treatment of AML with adverse cytogenetic risk, in particular of AML with adverse cytogenetic risk in patients with less than 20% blasts in the bone marrow.

The present invention discloses a specific chimeric antigen receptor (123 CAR) having comprising has at least 80% sequence identity with SEQ ID NO: 19, preferably humanized.

The present invention discloses a CD123 specific chimeric antigen receptor (CAR) comprising an extra cellular ligand binding-domain VH and VL from the monoclonal anti-CD123 antibody klon43 comprising the following CDR sequences:

(SEQ ID NO: 13)
GFTFTDYY,
(SEQ ID NO: 14)
RSKADGYTT,
(SEQ ID NO: 15)
ARDAAYYSYYSPEGAMDY,
and
(SEQ ID NO: 16)
QNVDSA,
(SEQ ID NO: 17)
SAS,
(SEQ ID NO: 18)
QQYYSTPWT,

In particular embodiments, the VH and VL are humanized.

The sequence of the CAR CD123 in the preferred invention is preferably as follows:

(SEQ ID NO: 1 + SEQ ID NO: 19)

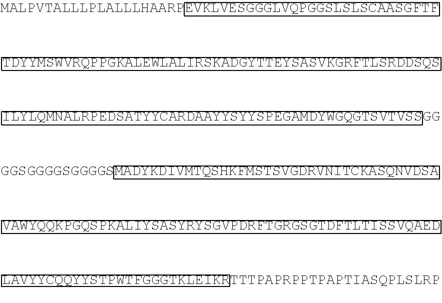

EACRPAAGGAVHTRGLDFACDIYIWAPLAGTCGVLLLSLVITLYCKRGRK
KLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR

Once at the cell surface said CAR is specific for CD123 and is preferably as follows:

(SEQ ID NO: 19)

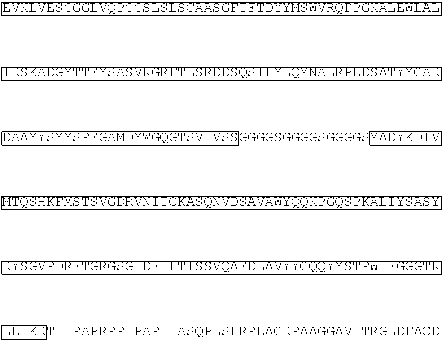

IYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGK
GHDGLYQGLSTATKDTYDALHMQALPPR.

The present invention also discloses a specific chimeric antigen receptor targeting the antigen CLL1 (CLL1 CAR) having preferably the structure presented in Table 3, and more preferably comprising a polypeptide sequence that has at least 80%, 90 or 95% sequence identity with the following one:

(SEQ ID NO: 35)
MALPVTALLLPLALLLHAARPEVQLQQSGPELVKPGASVKMSCKASG
YTFTSYFIHWVKQKPGQGLEWIGFINPYNDGSKYNEKFKGKATLTSD
KSSSTAYMELSSLTSEDSAVYYCTRDDGYYGYAMDYWGQGTSVTVSS
GGGGSGGGGSGGGGSDIQMTQSPSSLSASLGERVSLTCRATQELSGY
LSWLQQKPDGTIKRLIYAASTLDSGVPKRFSGNRSGSDYSLTISSLE
SEDFADYYCLQYAIYPYTFGGGTKLEIKSDPGSGGGGSCPYSNPSLC
SGGGGSCPYSNPSLCSGGGGSTTTPAPRPPTPAPTIASQPLSLRPEA
CRPAAGGAVHTRGLDFACDIYIWAPLAGTCGVLLLSLVITLYCKRGR
KKLLYIFKQPFMRPVQTTQEEDGCSCRFPEEEEGGCELRVKFSRSAD
APAYQQGQNQLYNELNLGRREEYDVLDKRRGRDPEMGGKPRRKNPQE
GLYNELQKDKMAEAYSEIGMKGERRRGKGHDGLYQGLSTATKDTYDA
LHMQALPPR.

Such CARs preferably comprise humanized versions of VH and VL. According to a preferred embodiment said CAR according to the invention comprises an additional sequence comprising an epitope referred to as R2, specifically recognized by an antibody allowing the immune depletion of the engineered CAR positive immune cells as described for instance in WO2016120216.

Cells or population of cells endowed with said CAR may be hematopoietic stem cells (HSC) to be derived into T cells or T cells, and comprise an intact alpha TCR gene if said cells is from the patient intended to be treated or a alpha TCR KO, preferably a TALEN®-mediated alpha TCR KO, even more preferably a TALEN® as disclosed below—mediated alpha TCR KO.

Cells or population of cells endowed with said CAR may be primary hematopoietic stem cells (HSC) to be derived into primary T cells or primary T cells, and comprise an intact alpha TCR gene if said cells is from the patient intended to be treated or comprise an alpha TCR KO gene, preferably a TALEN®—mediated alpha TCR KO gene, even more preferably a TALEN® as disclosed below—mediated alpha TCR KO genes (the two alleles are KO).

T cells or population of cells of the invention are functional memory T cells and/or non exhausted T cells (as defined in Wherry E J, Kurachi M. Molecular and cellular insights into T cell exhaustion. Nat Rev Immunol. 2015; 15(8):486-99.).

Engineered cells of the invention comprise at the cell surface a CAR as above, and at least one suicide domain, (R)n n is 0 to 10 and/or (Q)m m is 0 to 10 or RQR8. Preferably, engineered cells of the invention comprise a CAR at the cell surface and an exogenous sequence stably inserted into its genome encoding said CAR.

Compositions:

The present invention discloses a method of impairing a hematologic cancer comprising contacting said hematologic cancer with an engineered cell according to the present invention in an amount effective to cause impairment of said cancer cell, preferably at a dose selected from 2.5×10⁵/kg, 6.25×10⁵/kg, 5.05×10⁶/kg.

The term “extracellular ligand-binding domain” as used herein is defined as an oligo- or polypeptide that is capable of binding a ligand at the cell surface. Preferably, the domain will be capable of interacting with a cell surface molecule on cancer cells. For example, the extracellular ligand-binding domain may be chosen to recognize a ligand that acts as a cell surface marker on target cells associated with a particular disease state.

In a preferred embodiment, said extracellular ligand-binding domain comprises a single chain antibody fragment (scFv) comprising the light (V_{L}) and the heavy (V_{H}) variable fragment of a target antigen specific monoclonal anti CD-123 antibody Klon 43 joined by a flexible linker. Said V_{L} and V_{H} are preferably from Klon43 linked together by a flexible linker comprising the sequence SEQ ID NO.10.

By the term “recombinant antibody” as used herein, is meant an antibody or antibody fragment which is generated using recombinant DNA technology, such as, for example, an antibody or antibody fragment expressed by a bacteriophage, a yeast expression system or a mammalian cell expression system. The term should also be construed to mean an antibody or antibody fragment which has been generated by the synthesis of a DNA molecule encoding the antibody or antibody fragment and which DNA molecule expresses an antibody or antibody fragment protein, or an amino acid sequence specifying the antibody or antibody fragment, wherein the DNA or amino acid sequence has been obtained using recombinant or synthetic DNA or amino acid sequence technology which is available and well known in the art.

As used herein, the term “conservative sequence modifications” or “humanization” is intended to refer to amino acid modifications that do not significantly affect or alter the binding characteristics of the CAR and/or that do not significantly affect the activity of the CAR containing the modified amino acid sequence and reduce or abolish a human antimouse antibody (HAMA) response. Such conservative modifications include amino acid substitutions, additions and deletions in said antibody fragment in said CAR and/or any of the other parts of said CAR molecule. Modifications can be introduced into an antibody, into an antibody fragment or in any of the other parts of the CAR molecule of the invention by standard techniques known in the art, such as site-directed mutagenesis, PCR-mediated mutagenesis or by employing optimized germline sequences.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested for the ability to bind its target (eg: CD 123, CLL1 as non-limitatively described in the experimental section) using the functional assays described herein.

The signal transducing domain or intracellular signaling domain of a CAR according to the present invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector signal function signal and directs the cell to perform a specialized function.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the CAR can comprise the CD3zeta signaling domain which has amino acid sequence with at least 70%, preferably at least 80%, more preferably at least 90%, 95% 97% or 99% or 100% sequence identity with amino acid sequence selected from the group consisting of SEQ ID NO:9.

In particular embodiment the signal transduction domain of the CAR of the present invention comprises a co-stimulatory signal molecule. A co-stimulatory molecule is a cell surface molecule other than an antigen receptor or their ligands that is required for an efficient immune response. “Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83. A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83.

In a preferred embodiment, the signal transduction domain of the CAR of the present invention comprises a part of co-stimulatory signal molecule selected from the group consisting of fragment of 4-1BB (GenBank: AAA53133.) and CD28 (NP_006130.1). In particular the signal transduction domain of the CAR of the present invention comprises amino acid sequence which comprises amino acid sequence of SEQ ID NO: 8.

A CAR according to the present invention is expressed on the surface membrane of the cell. Thus, such CAR further comprises a transmembrane domain. The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T-cell receptor such as α, β, γ or □, polypeptide constituting CD3 complex, IL2 receptor p55 (α chain), p75 (β chain) or γ chain, subunit chain of Fc receptors, in particular Fcγ receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1) The transmembrane domain can further comprise a hinge region between said extracellular ligand-binding domain and said transmembrane domain. The term “hinge region” used herein generally means any oligo- or polypeptide that functions to link the transmembrane domain to the extracellular ligand-binding domain. In particular, hinge region is used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence, or may be an entirely synthetic hinge sequence. In a preferred embodiment said hinge domain comprises a part of human CD8 alpha chain, FcγRIIIα receptor or IgG1 respectively

A CAR according to the invention generally further comprises a transmembrane domain (TM) more particularly from CD8a, showing identity with the polypeptides of SEQ ID NO. 6 or 7.

Downregulation or mutation of target antigens is commonly observed in cancer cells, creating antigen-loss escape variants. Thus, to offset tumor escape and render immune cell more specific to target, the CD123 specific CAR according to the invention can comprise another extracellular ligand-binding domain, to simultaneously bind different elements in target thereby augmenting immune cell activation and function. In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the CAR. In another embodiment, the present invention relates to a population of CARs comprising each one different extracellular ligand binding domains. In a particular, the present invention relates to a method of engineering immune cells comprising providing an immune cell and expressing at the surface of said cell a population of CAR each one comprising different extracellular ligand binding domains. In another particular embodiment, the present invention relates to a method of engineering an immune cell comprising providing an immune cell and introducing into said cell polynucleotides encoding polypeptides composing a population of CAR each one comprising different extracellular ligand binding domains. By population of CARs, it is meant at least two, three, four, five, six or more CARs each one comprising different extracellular ligand binding domains. The different extracellular ligand binding domains according to the present invention can preferably simultaneously bind different elements in target thereby augmenting immune cell activation and function. The present invention also relates to an isolated immune cell which comprises a population of CARs each one comprising different extracellular ligand binding domains.

According to the invention, the immune cells expressing the anti-CD123 CAR and/or anti-CLL1 CAR of the invention trigger an anti-cancer immune response. In a preferred embodiment, the immune cells expressing the CAR of the invention endowed with the anti-CD123 CAR and/or anti-CLL1 CAR of the invention does trigger an immune response which does not comprise a human anti-mouse antibody (HAMA) response.

According to the invention, an efficient amount of the engineered immune cell can be administered to a patient in need thereof at least once, twice, or several times, in combination with a lymphodepleting treatment.

Polynucleotides, Vectors:

The present invention also relates to polynucleotides, vectors encoding the above described CAR according to the invention.

The polynucleotide may consist in an expression cassette or expression vector (e.g. a plasmid for introduction into a bacterial host cell, or a viral vector such as a recombinant lentivirus vector or an adeno associated vector for transfection of a mammalian host cell and stable integration of exogenous gene into their genome).

In particular embodiments, the different nucleic acid sequences can be included in one polynucleotide or vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptide into the secretory pathway of a host cell, a secretory signal sequence (also known as a leader sequence, pre- or pro-sequence or pre sequence) is provided in polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). In a preferred embodiment the signal peptide comprises the amino acid sequence SEQ ID NO: 1 and 2 or at least 90%, 95% 97% or 99% sequence identity with SEQ ID NO: 1 and/or 2.

Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

Cells

Cell according to the present invention refers to a cell of hematopoietic origin functionally involved in the initiation and/or execution of innate and/or adaptative immune response. Cell according to the present invention is preferably a T-cell obtained from a donor. Said T cell according to the present invention can be derived from a stem cell. The stem cells can be adult stem cells, embryonic stem cells, more particularly non-human stem cells, cord blood stem cells, progenitor cells, bone marrow stem cells, totipotent stem cells or hematopoietic stem cells. In a preferred embodiment, cells are human cells, in particular human stem cells.

Representative human stem cells are CD34+ cells. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+T-lymphocytes and CD8+T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T-cell lines available and known to those skilled in the art, may be used. In another embodiment, said cell is preferably derived from a healthy donor. In another embodiment, said cell is part of a mixed population of cells which present different phenotypic characteristics.

Preferably, isolation and preparation of stem cells does not require the destruction of at least one human embryo. The immune cells can originate from the patient, in view of operating autologous treatments, or from donors in view of producing allogeneic cells, which can be used in allogeneic treatments.

More preferably the immune cell of the invention express an anti-CD123 CAR corresponding to SEQ ID NO:19 and/or an anti-CLL1 CAR corresponding to SEQ ID NO: 35.

Methods of Engineering Immune Cells Endowed with CARs:

The present invention encompasses the method of preparing immune cells for immunotherapy comprising introducing ex-vivo into said immune cells the polynucleotides or vectors encoding the CAR (CD123CAR) previously described in WO2014/130635, WO2013176916, WO2018/073391, WO2013176915 and incorporated herein by reference.

In a preferred embodiment, said polynucleotides are included in lentiviral vectors in view of being stably expressed in the immune cells.

According to further embodiments, said method further comprises the step of genetically modifying said cell to make them more suitable for allogeneic transplantation.

In another preferred embodiment, said polynucleotides are included in AAV6 vectors in view of being stably expressed in the immune cells at the TCR alpha locus resulting in inactivation of the TCR and expression of CAR in the same cell.

### Modifying T-Cell by Inactivating at Least One Gene Encoding a T-Cell Receptor (TCR) Component.

According to a first aspect, the immune cell can be made less allogeneic, for instance, by inactivating at least one gene expressing one or more component of T-cell receptor (TCR) as described in WO 2013/176915, which can be combined with the inactivation of a gene encoding or regulating HLA or β2m protein expression. Accordingly, the risk of graft versus host syndrome and graft rejection is significantly reduced.

Accordingly, when the immune cells are T-cells, the present invention also provides methods to engineer T-cells that are less allogeneic.

Methods of making cells less allogenic comprise a step of inactivating at least one gene encoding a T-Cell Receptor (TCR) component, in particular TCRalpha, TCRbeta genes.

Methods disclosed in WO2013/176915 to prepare CAR expressing immune cell suitable for allogeneic transplantation, by inactivating one or more component of T-cell receptor (TCR), are all incorporated herein by reference.

The present invention encompasses an anti-CD123 CAR expressing immune cell wherein at least one gene expressing one or more component of T-cell receptor (TCR) has been inactivated. Thus, the present invention provides an anti-CD123 CAR expressing T cell wherein the CAR is derived from Klon 43, in particular having at least 80% identity with SEQ ID NO:19 and wherein at least one gene expressing one or more component of T-cell receptor (TCR) is inactivated.

According to the invention, anti-CD123 CAR immune cells with one or more component of T-cell receptor (TCR) inactivated, are intended to be used as a medicament for the treatment of hematopoietic cancer in patients with less than 0% blasts in the bone marrow.

By inactivating a TCR gene it is intended that the gene of interest is not expressed in a functional protein form. In particular embodiments, the genetic modification of the method relies on the expression, in provided cells to engineer, of one rare-cutting endonuclease such that said rare-cutting endonuclease specifically catalyzes cleavage in one targeted gene thereby inactivating said targeted gene. The nucleic acid strand breaks caused by the rare-cutting endonuclease are commonly repaired through the distinct mechanisms of homologous recombination or non-homologous end joining (NHEJ). However, NHEJ is an imperfect repair process that often results in changes to the DNA sequence at the site of the cleavage. Mechanisms involve rejoining of what remains of the two DNA ends through direct re-ligation [Critchlow and Jackson (1998) DNA end-joining: from yeast to man. Trends in Biochemical Sciences 23(10):394-398] or via the so-called microhomology-mediated end joining [Ma, J. L., (2003) Yeast Mre11 and Rad1 Proteins Define a Ku-Independent Mechanism. Molecular and Cellular Biology. 23(23): 8820-8828].

To Repair Double-Strand Breaks Lacking Overlapping End SequencesRepair via non-homologous end joining (NHEJ) often results in small insertions or deletions and can be used for the creation of specific gene knockouts. Said modification may be a substitution, deletion, or addition of at least one nucleotide. Cells in which a cleavage-induced mutagenesis event, i.e. a mutagenesis event consecutive to an NHEJ event, has occurred can be identified and/or selected by well-known method in the art. In a particular embodiment, the step of inactivating at least a gene encoding a component of the T-cell receptor (TCR) into the cells of each individual sample comprises introducing into the cell a rare-cutting endonuclease able to disrupt at least one gene encoding a component of the T-cell receptor (TCR). In a more particular embodiment, said cells of each individual sample are transformed with nucleic acid encoding a rare-cutting endonuclease capable of disrupting at least one gene encoding a component of the T-cell receptor (TCR), and said rare-cutting endonuclease is expressed into said cells.

Said rare-cutting endonuclease can be a meganuclease, a Zinc finger nuclease, CRISPR/Cas9 nuclease, Argonaute nuclease, a TALE-nuclease or a MBBBD-nuclease. In a preferred embodiment, said rare-cutting endonuclease is a TALE-nuclease. By TALE-nuclease is intended a fusion protein consisting of a DNA-binding domain derived from a Transcription Activator Like Effector (TALE) and one nuclease catalytic domain to cleave a nucleic acid target sequence [Mussolino & Cathomen (2012) TALE-nucleases: tailored genome engineering made easy. Current Opinion in Biotechnology. 23(5):644-650]. In the present invention new TALE-nucleases have been designed for precisely targeting relevant genes for adoptive immunotherapy strategies.

Preferred TALE-nucleases recognizing and cleaving the target sequence are described in PCT/EP2014/075317. In particular, additional catalytic domain can be further introduced into the cell with said rare-cutting endonuclease to increase mutagenesis in order to enhance their capacity to inactivate targeted genes. More particularly, said additional catalytic domain is a DNA end processing enzyme. Non limiting examples of DNA end-processing enzymes include 5-3′ exonucleases, 3-5′ exonucleases, 5-3′ alkaline exonucleases, 5′ flap endonucleases, helicases, hosphatase, hydrolases and template-independent DNA polymerases. Non limiting examples of such catalytic domain comprise of a protein domain or catalytically active derivate of the protein domain selected from the group consisting of hExol (EXO1_HUMAN), Yeast Exol (EXO1_YEAST), E. coli Exol, Human TREX2, Mouse TREX1, Human TREX1, Bovine TREX1, Rat TREX1, TdT (terminal deoxynucleotidyl transferase) Human DNA2, Yeast DNA2 (DNA2_YEAST). In a preferred embodiment, said additional catalytic domain has a 3′-5′-1.0 exonuclease activity, and in a more preferred embodiment, said additional catalytic domain is TREX, more preferably TREX2 catalytic domain (WO2012/058458). In another preferred embodiment, said catalytic domain is encoded by a single chain TREX2 polypeptide. Said additional catalytic domain may be fused to a nuclease fusion protein or chimeric protein according to the invention optionally by a peptide linker.

Endonucleolytic breaks are known to stimulate the rate of homologous recombination. Thus, in another embodiment, the genetic modification step of the method further comprises a step of introduction into cells of an exogeneous nucleic acid comprising at least a sequence homologous to a portion of the target nucleic acid sequence, such that homologous recombination occurs between the target nucleic acid sequence and the exogeneous nucleic acid. In particular embodiments, said exogenous nucleic acid comprises first and second portions which are homologous to region 5′ and 3′ of the target nucleic acid sequence, respectively. Said exogenous nucleic acid in these embodiments also comprises a third portion positioned between the first and the second portion which comprises no homology with the regions 5′ and 3′ of the target nucleic acid sequence. Following cleavage of the target nucleic acid sequence, a homologous recombination event is stimulated between the target nucleic acid sequence and the exogenous nucleic acid. Preferably, homologous sequences of at least 50 bp, preferably more than 100 bp and more preferably more than 200 bp are used within said donor matrix. In a particular embodiment, the homologous sequence can be from 200 bp to 6000 bp, preferably from 1000 bp to 2000 bp and more preferably from 300 bp to 1000 bp. Indeed, shared nucleic acid homologies are located in regions flanking upstream and downstream the site of the break and the nucleic acid sequence to be introduced should be located between the two arms.

Immune Check Points

The present invention provides allogeneic T-cells expressing an anti-CD123 CAR, in particular an anti-CD123 CAR of SEQ ID N^{o} 19 or of SEQ ID NO:1+ SEQ ID NO:19, wherein at least one gene expressing one or more component of T-cell receptor (TCR) is inactivated and/or one gene selected from the genes CTLA4, PPP2CA, PPP2CB, PTPN6, PTPN22, PDCD1, LAG3, HAVCR2, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, TGFBRII, TGFBRI, SMAD2, SMAD3, SMAD4, SMAD10, SKI, SKIL, TGIF1, IL10RA, IL10RB, HMOX2, IL6R, IL6ST, CSK, PAG1, SIT1, FOXP3, PRDM1 (orblimp1), BATF, GUCY1A2, GUCY1A3, GUCY1B2, GUCY1B3, is inactivated as referred to in WO2014/184741.

Drug Resistant T-Cells

According to another aspect, the anti-CD123 CAR expressing T-cell of the invention can be further genetically engineered to improve its resistance to immunosuppressive drugs or chemotherapy treatments, which are used as standard care for treating CD123 positive malignant cells.

Several cytotoxic agents (anti-cancer drugs) such as anti-metabolites, alkylating agents, anthracyclines, DNA methyltransferase inhibitors, platinum compounds and spindle poisons have been developed to kill cancer cells. However, the introduction of these agents with novel therapies, such as immunotherapies, is problematic. For example, chemotherapy agents can be detrimental to the establishment of robust anti-tumor immunocompetent cells due to the agents' non-specific toxicity profiles. Small molecule-based therapies targeting cell proliferation pathways may also hamper the establishment of anti-tumor immunity. If chemotherapy regimens that are transiently effective can be combined with novel immunocompetent cell therapies then significant improvement in anti-neoplastic therapy might be achieved (for review [Dasgupta, A. et al. (2012) Treatment of a Solid Tumor Using Engineered Drug-Resistant Immunocompetent Cells and Cytotoxic Chemotherapy. Human Gene Therapy 23(7):711-721].

To improve cancer therapy and selective engraftment of allogeneic T-cells, drug resistance is conferred to said allogeneic T cells to protect them from the toxic side effects of chemotherapy agent. The drug resistance of T-cells also permits their enrichment in or ex vivo, as T-cells which express the drug resistance gene will survive and multiply relative to drug sensitive cells.

Methods for engineering T-cells resistant to chemotherapeutic agents are disclosed in PCT/EP2014/075317 which is fully incorporated by reference herein.

In particular, the present invention relates to a method of engineering allogeneic cells suitable for immunotherapy wherein at least one gene encoding a T-cell receptor (TCR) component is inactivated and one gene is modified to confer drug resistance comprising:
Providing an anti-CD123 and/or anti-CLL1 CAR expressing T-cell; in particular an anti-CD123 CAR of SEQ ID NO:19, expressing T cell, preferably humanized 123 CAR of SEQ ID NO:19,
inactivating at least one gene encoding a T-cell receptor (TCR) component;
inactivating the CD52 gene;
to confer resistance anti-CD52 therapeutic antibody Campath (alemtuzumab) to said anti-CD123 CAR expressing T-cell;
Expanding said engineered anti-CD123 and/or anti-CLL1 CAR expressing T-cell in the presence of said drug Campath (alemtuzumab).

Alternatively, the present invention relates to a method comprising:
Providing an anti-CD123 and/or anti-CLL1 CAR expressing T-cell; in particular an anti-CD123 CAR of SEQ ID NO:19, expressing T cell, preferably humanized
inactivating the CD52 gene to confer resistance to Campath (alemtuzumab)
inactivating at least one gene encoding a T-cell receptor (TCR) component;
Expanding said engineered anti-CD123 and/or anti-CLL1 CAR expressing T-cell in the presence of said drug Campath (alemtuzumab).

Suicide Genes in Anti-CD123 CAR-Expressing Immune Cells

In some instances, since engineered T-cells can expand and persist for years after administration, it can be desirable to include a safety mechanism to allow selective deletion of administrated T-cells. Thus, in some embodiments, the method of the invention can comprise the transformation of said T-cells with a recombinant suicide gene. Said recombinant suicide gene is used to reduce the risk of direct toxicity and/or uncontrolled proliferation of said T-cells once administrated in a subject [Quintarelli C, Vera F, (2007) Co-expression of cytokine and suicide genes to enhance the activity and safety of tumor-specific cytotoxic T lymphocytes Blood. 10(8):2793]. Suicide genes enable selective deletion of transformed cells in vivo. In particular, the suicide gene has the ability to convert a non-toxic pro-drug into cytotoxic drug or to express the toxic gene expression product. In other words, “Suicide gene” is a nucleic acid coding for a product, wherein the product causes cell death by itself or in the presence of other compounds.

One preferred suicide gene system employs in the present invention is a recombinant antigenic polypeptide comprising motifs recognized by the anti-CD20 mAb Rituximab, and by the anti-CD34, QBen10, such as in the so-called RQR8 polypeptide described in WO2013153391.

In other embodiments, the extracellular domain of the CD123 CAR and/or anti-CLL1 comprises at least one epitope recognized by Rituximab, and rituximab can then be used alone or in combination with QBen10, when needed, in combination with the composition of the invention. Also, the extracellular domain of the CD123 CAR and/or anti-CLL1 can comprise at least one epitope recognized by Rituximab as described in WO2016120216, and rituximab can then be used at a dose of 375 mg/m² weekly, or at a dose of 375 mg/m² weekly for up to 4 weeks.

In one embodiment, the present invention provides allogenic anti-CD123 CAR and/or anti-CLL1 expressing T-cell expressing more than one drug resistance gene or wherein more than one drug sensitizing gene is inactivated, and a suicide gene allowing said cells to be destroyed.

### Clofarabine Resistant Anti-CD123 CAR-Expressing Immune Cells

The invention encompasses the manufacture of T cells for therapeutic use, which are resistant a drug such as to Clofarabine. They can be obtained by inactivation of the dCK gene such as previously explained. According to a preferred embodiment, the T-cells are made resistant to chemotherapy and less allogeneic by combining inactivation of dCK and TCR genes as previously described.

Thus, the present invention provides an anti-CD123 CAR and/or anti-CLL1 expressing cell, in particular an anti-CD123 CAR expressing T cell wherein the CAR is derived from Klon 43 (comprising a SEQ ID NO:19, optionally humanized) and wherein the dCK gene is inactivated.

The present invention encompasses also a method for manufacturing target cells which express both a surface receptor specific to the CAR T cells and a resistance gene. These target cells are particularly useful for testing the cytotoxicity of CAR T cells. These cells are readily resistant to clinically relevant dose of clofarabine and harbor luciferase activity. This combination of features enable traking them in vivo in a mice model or destroy them when required.

More particularly, they can be used to assess the cytotoxicity properties drug resistant T cells in mice in the presence of clofarabine or other PNAs. Clofarabine resistant Daudi cells mimick the physiological state of acute lymphoblastic leukemia (ALL) patients relapsing form induction therapy, that harbor drug resistant B cell malignancies. Thus, these cells are of great interest to evaluate the reliability and cytotoxicity of drug resistant CAR T cells. Preferably, these target cells are CD123+ Luciferase+ Daudi cells.

The immune cells of the present invention or cell lines can further comprise exogenous recombinant polynucleotides, in particular CARs or suicide genes or they can comprise altered or deleted genes coding for checkpoint proteins or ligands thereof that contribute to their efficiency as a therapeutic product, ideally as an “off the shelf” product. In another aspect, the present invention concerns the method for treating or preventing cancer in the patient by administrating at least once an engineered immune cell obtainable by the above methods.

Delivery Methods

The different methods described above involve expressing a protein of interest such as drug resistance gene, rare-cutting endonuclease, Chimeric Antigen Receptor (CAR), in particular an anti-CD123 CAR and more particularly, a CAR comprising a SEQ ID NO. 1+ SEQ ID NO. 19, and a suicide gene encoding a RQR8, into a cell.

By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

In a more preferred embodiment of the invention, polynucleotides encoding polypeptides according to the present invention can be mRNA which is introduced directly into the cells, for example by electroporation. The inventors determined the optimal condition for mRNA electroporation in T-cell. The inventor used the cytoPulse technology which allows, by the use of pulsed electric fields, to transiently permeabilize living cells for delivery of material into the cells. The technology, based on the use of PulseAgile (BTX Havard Apparatus, 84 October Hill Road, Holliston, Mass. 01746, USA) electroporation waveforms grants the precise control of pulse duration, intensity as well as the interval between pulses (U.S. Pat. No. 6,010,613 and International PCT application WO2004083379). All these parameters can be modified in order to reach the best conditions for high transfection efficiency with minimal mortality. Basically, the first high electric field pulses allow pore formation, while subsequent lower electric field pulses allow moving the polynucleotide into the cell.

The different methods described above involve introducing CAR into a cell. As non-limiting example, said CAR can be introduced as transgenes encoded by one plasmid vector. Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

Activation and Expansion of T Cells

Whether prior to or after genetic modification of the T cells, even if the genetically modified immune cells of the present invention are activated and proliferate independently of antigen binding mechanisms, the immune cells, particularly T-cells of the present invention can be further activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

Generally, the T cells of the invention are expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, -2, 1L-15, TGFbeta, and TNF- or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics.

Engineered Immune Cells

The present invention relates to genetically modified immune cells (engineered immune cells). Engineered immune cells means cells expressing a CAR, at the cell surface.

In particular embodiments, engineered immune cells were isolated from the patient intended to be treated (autologous transfer). In that case, engineered immune cells may further be engineered to be resistant to particular drugs used in the composition of the invention such as fludarabine or other PNA. Cells may therefore comprise an inactivated dck gene, a CD52 inactivated gene and express a suicide gene.

In the scope of the present invention is also encompassed an isolated immune cell, preferably a T-cell obtained according to any one of the methods previously described. Said immune cell refers to a cell of hematopoietic origin functionally involved in the initiation and/or execution of innate and/or adaptative immune response. Said immune cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

Said isolated cell can also be a dendritic cell, killer dendritic cell, a mast cell, a NK-cell, a B-cell or a T-cell, preferably a T cell selected from the group consisting of inflammatory T-lymphocytes, cytotoxic T-lymphocytes, regulatory T-lymphocytes or helper T-lymphocytes.

In another embodiment, said cell can be derived from the group consisting of CD4+T-lymphocytes and CD8+T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used. In another embodiment, said cell can be derived from a healthy donor, from a patient diagnosed with cancer. In another embodiment, said cell is part of a mixed population of cells which present different phenotypic characteristics.

Modified cells resistant to an immunosuppressive -(alemtuzumab) treatment and susceptible to be obtained by the previous method are encompassed in the scope of the present invention.

The engineered cells in the composition of the invention express a CAR at the cell surface specific for a tumoral antigen selected from CD25, CD30, CD37, CD38, CD33, CD47, CD98, CD123, FLT3, CLL-1, CD56, CD117, CD133, CD157, c-kit, CD34, MUC1, CXCR4, VEGF, NKG2D_F, folate receptor beta (FR beta), hepatocyte growth factor (HGF), HLA-A2, human C-type lectin-like molecule-1 (CLL1), Lewis Y, a combination thereof, preferably specific for CD123 [in Universal CAR T targeting CD123 (UCART123)] and/or human C-type lectin-like molecule-1 (CLL1).

The engineered cells in the composition of the invention express a CAR at the cell surface specific for a tumoral antigen which is any one of the following: CD25, CD30, CD37, CD38, CD33, CD47, CD98, CD123, FLT3, CLL-1, CD56, CD117, CD133, CD157, c-kit, CD34, MUC1, CXCR4, VEGF, NKG2D_F, folate receptor beta (FR beta), hepatocyte growth factor (HGF), HLA-A2, human C-type lectin-like molecule-1 (CLL1), Lewis Y, a combination thereof, preferably specific for CD123[in Universal CAR T targeting CD123 (UCART123)] and/or human C-type lectin-like molecule-1 (CLL1).

The CAR may comprise two tumor antigen binding domains in addition to a domain binding to CD123 selected from CD25, CD30, CD37, CD38, CD33, CD47, CD98, FLT3, CLL-1, CD56, CD117, CD133, CD157, c-kit, CD34, MUC1, CXCR4, VEGF, NKG2D_F, folate receptor beta (FR beta), hepatocyte growth factor (HGF), HLA-A2, human C-type lectin-like molecule-1 (CLL1), Lewis Y.

As a preferred embodiment, the present invention provides T-cells or a population of T-cells endowed with a CD123 CAR and/or anti-CLL1 CAR as described above, that do not express functional TCR and that a reactive towards CD123 and/or CLL1 positive cells, for their allogeneic transplantation into patients (UCART 123 and/or UCART CLL1).

As a preferred embodiment, the present invention provides T-cells or a population of T-cells endowed with a CD123 CAR or CLL1 CAR as described above, expressing a suicide gene RQR8 or R2 or QR3 that do not express functional TCR and that a reactive towards CD123 and/or CLL1 positive cells, for their allogeneic transplantation into patients (UCART 123). A combination of said cells with QBEN10 and/or Rituximab is contemplated.

As a more preferred embodiment, the present invention provides T-cells or a population of T-cells endowed with a CD123 CAR and/or CLL1 CAR and that a reactive towards CD123 positive cells as described above, that do not express a functional TCR and are resistant to alemtuzumab, (that do not express CD52) for their allogeneic transplantation into patients treated with said selected drug. A combination of said cells with alemtuzumab is contemplated.

As another more preferred embodiment, the present invention provides T-cells or a population of T-cells endowed with a CD123 CAR and/r CLL1 CAR and that a reactive towards CD123 positive cells as described above, that do not express a functional TCR and are resistant to alemtuzumab, (e.g. due to impaired expression of CD52) and that express a suicide gene for their allogeneic transplantation into patients treated with said selected drug.

The invention further provides with a combination comprising a lymphodepleting treatment and at one dose of UCART for the treatment of a patient with cancer, especially AML with adverse genetic risk, while the patient has less than 20% blasts over total cells in the bone marrow. The invention also provides with a combination comprising a lymphodepleting treatment and at one dose of 5×10⁶/kg UCART for the treatment of a patient with hematological cancer and less than 20% blasts over total cells in the bone marrow.

The invention provides a combination comprising a lymphodepleting treatment and at one dose of 5.05×106/kg UCART for the treatment of a patient with hematological cancer, especially AML with adverse genetic risk, while the patient has less than 20% blasts over total cells in the bone marrow, and wherein the lymphodepleting treatment comprises fludarabine and Cyclophosphamide, preferably fludarabine 30 mg/m²/day from Day −5 to Day −2 with a maximum daily dose of 60 mg; Cyclophosphamide 1 g/m²/day from Day −4 to Day −2 with a maximum daily dose of 2 grams.

The combination below is especially active in AML with adverse genetic in patient with less than 20% blasts in the bone marrow.

Debulking Treatment

In the combination of the present invention, the UCART can be administered after a debulking treatment. Debulking is meant to be the reduction of as much of the bulk (volume) of the tumor as possible. The criteria set up in the present invention was less than 20% blasts in the bone marrow.

In the present invention the debulking treatment is achieved by cytoreduction “cytoreduction” and refers to reducing the number of tumor cells, with palliative intent to relieve mass effect and prevent cytokine storm or cytokine releasing syndrome during treatment with UCART cells.

A debulking treatment generally comprises cytarabine and optionally idarubicin and/or azacytidine. In a preferred embodiment, a debulking treatment comprises anthracycline, daunorubicin, idarubicin or mitoxantrone and cytarabine, a combination thereof.

FLAG is an acronym for a chemotherapy regimen comprising:
1. Fludarabine
2. cytarabine (Arabinofuranosyl cytidine, or ara-C):
3. Granulocyte colony-stimulating factor (G-CSF): a glycoprotein that shortens the duration and severity of neutropenia.

As an example of debulking treatment: a FLAG-IDA, MITO-FLAG and FLAMSA regimen may be used.

### FLAG-IDA

In the FLAG-IDA regimen (also called FLAG-Ida, IDA-FLAG, or Ida-FLAG), idarubicin—an anthracycline antibiotic that is able to intercalate DNA and prevent cell division (mitosis) is added to the standard FLAG regimen.

### MITO-FLAG

MITO-FLAG (also called Mito-FLAG, FLAG-MITO, or FLAG-Mito) adds mitoxantrone to the standard FLAG regimen. Mitoxantrone is a synthetic anthracycline analogue (an anthracenedione) that, like idarubicin, can intercalate DNA and prevent cell division

### FLAMSA

FLAMSA adds amsacrine (“AMSA”) to the standard FLAG regimen. (G-CSF is still included, even though the “G” is taken out of the acronym). Amsacrine is an alkylating antineoplastic agent that is highly active toward AML, unlike more conventional alkylators like cyclophosphamide.
The FLAMSA protocol may be used as an induction part of a reduced-intensity conditioning regimen for patients eligible to undergo an allogeneic transplant with UCART. In this setting, it may be combined with other agents, such as:
Cyclophosphamide (FLAMSA-CYC), and/or
Busulfan or treosulfan (FLAMSA-BU or FLAMSA-TREO), and/or
Melphalan (FLAMSA-MEL), and/or
Total body irradiation, given shortly after the end of FLAMSA to prepare the patient for transplant.
   Examples of Dosing are as followed

TABLE 4
Standard FLAG

| Drug | Dose | | Mode | Days |
|---|---|---|---|---|
| (FL)udarabine | 30 mg/m2 a | | IV infusion over 30 min, every 12 hours | Days 1-5 |
| | day | | in 2 divided doses | |
| (A)ra-C | 2000 | mg/m2 | IV infusion over 4 hours, every 12 hours | Days 1-5 |
| | | | in 2 divided doses, starting 4 hours after | |
| | | | the end of fludarabine infusion | |
| (G)-CSF | 5 | μg/kg | SC | From day 6 until |
| | | | | neutrophil |
| | | | | recovery |

TABLE 5
FLAG-IDA

| Drug | Dose | | Mode | Days |
|---|---|---|---|---|
| (FL)udarabine | 30 mg/m2 a | | IV infusion over 30 min, every 12 hours in 2 | Days 1-5 |
| | day | | divided doses | |
| (A)ra-C | 2000 mg/m2 | | IV infusion over 4 hours, every 12 hours in 2 | Days 1-5 |
| | a day | | divided doses, starting 4 hours after the end | |
| | | | of fludarabine infusion | |
| (IDA)rubicin | 10 | mg/m2 | IV bolus | Days 1-3 |
| (G)-CSF | 5 | μg/kg | SC | From day 6 until |
| | | | | neutrophil |
| | | | | recovery |

TABLE 6
Mito-FLAG

| Drug | Dose | | Mode | Days |
|---|---|---|---|---|
| (FL)udarabine | 30 | mg/m2 | IV infusion over 30 min, every 12 hours in | Days 1-5 |
| | | | 2 divided doses | |
| (A)ra-C | 2000 | mg/m2 | IV infusion over 3 hours, every 12 hours in | Days 1-5 |
| | | | 2 divided doses, starting 4 hours after the | |
| | | | end of fludarabine infusion | |
| (Mito)xantrone | 7 | mg/m2 | IV infusion | Days 1, 3 and 5 |
| (G)-CSF | 5 | μg/kg | SC | From day 6 until |
| | | | | neutrophil |
| | | | | recovery |

TABLE 7
FLAMSA

| Drug | Dose | | Mode | Days |
|---|---|---|---|---|
| (FL)udarabine | 30 | mg/m2 | IV infusion over 30 min, every | Days 1-4 |
| | | | 12 hours in 2 divided doses | |
| (A)ra-C | 2000 | mg/m2 | IV infusion over 4 hours, every | Days 1-4 |
| | | | 12 hours in 2 divided doses, | |
| | | | starting 4 hours after the end of | |
| | | | fludarabine infusion | |
| (AMSA)crine | 100 | mg/m2 | IV infusion | Days 1-4 |
| Filgrastim | 5 | μg/kg | SC | From transplant day (or Day 5 |
| | | | | if FLAMSA is not a part of |
| | | | | conditioning) until neutrophil |
| | | | | recovery |

In preferred embodiments, the FLAG-Ida regimen comprises, or consists of, fludarabine 30 mg/m² from Day 2 to Day 6, cytarabine 1500-2000 mg/m² IV from Day 2 to Day 6; idarubicin 10 mg/m² from Day 2 to Day 4.

In other embodiments, the debulking treatment may be a “3+7” regimen. 7+3” chemotherapy regimen consists of 7 days of standard-dose cytarabine, and 3 days of an anthracycline antibiotic or an anthracenedione, most often daunorubicin (can be substituted for doxorubicin or idarubicin or mitoxantrone).

This 7+3 regimen generally comprises anthracycline, daunorubicin, idarubicin or mitoxantrone for 3 days and 7 days of continuous infusion cytarabine.

Dosing Regimen

TABLE 8
Standard-dose cytarabine plus daunorubicin
(DA or DAC chemotherapy)

| Drug | Dose | | Mode | Days |
|---|---|---|---|---|
| Cytarabine | 100-200 | mg/m2 | IV continuous infusion | Days 1-7 |
| | | | over 24 hours | |
| Daunorubicin | (45) 60-90 | mg/m2 | IV bolus | Days 1-3 |

TABLE 9
Standard-dose cytarabine plus idarubicin
(IA or IAC chemotherapy)

| Drug | Dose | | Mode | Days |
|---|---|---|---|---|
| Cytarabine | 100-200 | mg/m2 | IV continuous infusion | Days 1-7 |
| | | | over 24 hours | |
| Idarubicin | 12 | mg/m2 | IV bolus | Days 1-3 |

TABLE 10
Standard-dose cytarabine plus mitoxantrone
(MA or MAC chemotherapy)

| Drug | Dose | | Mode | Days |
|---|---|---|---|---|
| Cytarabine | 100-200 | mg/m2 | IV continuous infusion | Days 1-7 |
| | | | over 24 hours | |
| Mitoxantrone | 7 | mg/m2 | IV infusion | Days 1, 3 |
| | | | | and 5 |

Intensified Versions

“7+3” regimen duration and doses can be prolonged or reduced (e.g.: cytarabine for 10 days instead of 7, or daunorubicin/idarubicin for 4-5 days instead of 3).

The addition of vinca alkaloids (vincristine or vinblastine) to the “7+3” regimen, is proscribed Nevertheless because vinca alkaloids in the context of AML cause AML cells to undergo a cell cycle arrest in the phase, vinca may be used to make cells more sensitive to UCART.

Preferably; a “3+7” regimen comprises 3 days of an IV anthracycline: daunorubicin at least 60 mg/m²; idarubicin 12 mg/m²; or mitoxantrone 12 mg/m², and 7 days of continuous infusion cytarabine (100-200 mg/m²)).

The invention provides therefore a combination comprising:
at least one immunotherapy treatment comprising a lymphodepleting treatment followed by a dose of CART cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation

The invention further provides a combination comprising:
at least one debulking treatment or two debulking treatments for reducing the amount of blasts cells to less than 20% in the bone marrow, and
at least one immunotherapy treatment comprising a lymphodepleting treatment followed by a dose of CART cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation

An immunotherapy treatment comprises a lymphodepleting treatment followed by one dose of CART or UCART, in particular CART123 or UCART123

The composition of the present invention is used for the treatment of a patient with hematological cancer and less than 20% blasts over total cells in the bone marrow.

According to an embodiment, the invention provides a combination comprising:
at least one debulking treatment or two debulking treatments for reducing the amount of blasts cells to less than 20% in the bone marrow,
at least one immunotherapy treatment comprising a lymphodepleting treatment followed by a dose of CART cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%,
hematopoietic stem cells for transplantation

According to an embodiment, the invention provides a combination comprising:
at least one debulking treatment or two debulking treatments comprising either a “3+7” regimen (consisting of 3 days of an IV anthracycline: daunorubicin at least 60 mg/m²; idarubicin 12 mg/m²; or mitoxantrone 12 mg/m², and 7 days of continuous infusion cytarabine (100-200 mg/m²)); or a FLAG-Ida regimen (for example consisting of fludarabine 30 mg/m² from Day 2 to Day 6, cytarabine 1500-2000 mg/m² IV, Day 2 to Day 6; idarubicin 10 mg/m², Day 2 to Day 4); for reducing the amount of blasts cells to less than 20% in the bone marrow,
at least one immunotherapy treatment comprising a lymphodepleting treatment followed by a dose of CART cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%,
hematopoietic stem cells for transplantation.

According to an embodiment, the invention provides a combination comprising:
at least one debulking treatment or two debulking treatments comprising either a “3+7” regimen (consisting of 3 days of an IV anthracycline: daunorubicin at least 60 mg/m²; idarubicin 12 mg/m²; or mitoxantrone 12 mg/m², and 7 days of continuous infusion cytarabine (100-200 mg/m²)); or a FLAG-Ida regimen (for example consisting of fludarabine 30 mg/m² from Day 2 to Day 6, cytarabine 1500-2000 mg/m² IV, Day 2 to Day 6; idarubicin 10 mg/m², Day 2 to Day 4); for reducing the amount of blasts cells to less than 20% in the bone marrow,
at least one immunotherapy treatment comprising a lymphodepleting treatment followed by a dose of CART 123 cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation

According to an embodiment, the invention provides a combination comprising:
at least one debulking treatment or two debulking treatments for reducing the amount of blasts cells to less than 20% in the bone marrow,
at least one immunotherapy treatment comprising a lymphodepleting treatment comprising fludarabine 30 mg/m²/day IV for 4 days over 15 to 30 minutes from Day −5 to Day −2 with a maximum daily dose of 60 mg, and cyclophosphamide 1 g/m²/day IV over 1 hour for 3 days from Day −4 to Day −2 with a maximum daily dose of 2 grams, followed by a dose of CART cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation

According to an embodiment, the invention provides a combination comprising:
at least one debulking treatment or two debulking treatments comprising either a “3+7” regimen (consisting of 3 days of an IV anthracycline: daunorubicin at least 60 mg/m²; idarubicin 12 mg/m²; or mitoxantrone 12 mg/m², and 7 days of continuous infusion cytarabine (100-200 mg/m²)); or a FLAG-Ida regimen (for example consisting of fludarabine 30 mg/m² from Day 2 to Day 6, cytarabine 1500-2000 mg/m² IV, Day 2 to Day 6; idarubicin 10 mg/m², Day 2 to Day 4); for reducing the amount of blasts cells to less than 20% in the bone marrow,
at least one immunotherapy treatment comprising a lymphodepleting treatment comprising fludarabine 30 mg/m²/day IV for 4 days over 15 to 30 minutes from Day −5 to Day −2 with a maximum daily dose of 60 mg, and cyclophosphamide 1 g/m²/day IV over 1 hour for 3 days from Day −4 to Day −2 with a maximum daily dose of 2 grams, followed by a dose of CART cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation.

According to an embodiment, the invention provides a combination comprising:
at least one debulking treatment or two debulking treatments comprising either a “3+7” regimen (consisting of 3 days of an IV anthracycline: daunorubicin at least 60 mg/m²; idarubicin 12 mg/m²; or mitoxantrone 12 mg/m², and 7 days of continuous infusion cytarabine (100-200 mg/m²)); or a FLAG-Ida regimen (for example consisting of fludarabine 30 mg/m² from Day 2 to Day 6, cytarabine 1500-2000 mg/m² IV, Day 2 to Day 6; idarubicin 10 mg/m², Day 2 to Day 4); for reducing the amount of blasts cells to less than 20% in the bone marrow,
at least one immunotherapy treatment comprising a lymphodepleting treatment comprising fludarabine 30 mg/m²/day IV for 4 days over 15 to 30 minutes from Day −5 to Day −2 with a maximum daily dose of 60 mg, and cyclophosphamide 1 g/m²/day IV over 1 hour for 3 days from Day −4 to Day −2 with a maximum daily dose of 2 grams, followed by a dose of CART 123 cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation.

According to an embodiment, the invention provides a combination comprising:
at least one debulking treatment or two debulking treatments for reducing the amount of blasts cells to less than 20% in the bone marrow,
at least one immunotherapy treatment comprising a lymphodepleting treatment followed by a dose of CART 123 cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation

According to an embodiment, the invention provides a combination comprising:
at least one debulking treatment or two debulking treatments for reducing the amount of blasts cells to less than 20% in the bone marrow,
at least one immunotherapy treatment comprising a lymphodepleting treatment comprising fludarabine 30 mg/m²/day IV for 4 days over 15 to 30 minutes from Day −5 to Day −2 with a maximum daily dose of 60 mg, and cyclophosphamide 1 g/m²/day IV over 1 hour for 3 days from Day −4 to Day −2 with a maximum daily dose of 2 grams, followed by a dose of CART 123 cells, for reaching Complete Remission with Minimal Residual Disease <0.01% (by flow cytometry or molecular methods); and optionally a second immunotherapy treatment if the first was active but did not allow Complete Remission with Minimal Residual Disease <0.01%, and
hematopoietic stem cells for transplantation

Therapeutic Applications

The composition of the present invention may be used as a medicament.

Patients who can benefit from the composition of the invention may be newly diagnosed with CD123 and/or CLL1 positive adverse genetic risk acute myeloid leukaemia (AML), including patients with CD123 positive AML secondary to MDS, who do not achieve morphologic or cytogenetic complete remission, and whose bone marrow blast content is <20% blasts after no, 1 or 2 courses of standard intensive induction chemotherapy.

Adverse genetic risk is defined as per ELN guidelines (below, Döhner et al., 2017):
i. Inv(3)(q21.3q26.2) or t(3;3)(q21.3;q26.2); GATA2, MECOM(EVI1); or
ii. Complex karyotype (Three or more unrelated chromosome abnormalities in the absence of one of the World Health Organization-designated recurring translocations or inversions, i.e., t(8;21), inv(16) or t(16;16), t(9;11), t(v;11)(v;q23.3); AML with BCR-ABL1); or
iii. Monosomal karyotype (presence of one single monosomy (excluding loss of X or Y) in association with at least one additional monosomy or structural chromosome abnormality (excluding core-binding factor AML); or
iv. Mutated TP53 with VAF >10%.

The invention provides therefore a combination as any one of the above for treating cancer, particularly for the treatment of hematological cancer such as B-cell lymphoma and leukemia in a patient in need thereof with less than 20% blasts over total cells in the bone marrow.

In a particular embodiment, an anti-CD123 CAR expressing T cell CART 123 or UCART 123 is provided as a medicament for the treatment of AML, of an AML with adverse cytogenetic risk as defined in Döhner, H., Estey, E., Grimwade, D., Amadori, S., Appelbaum, F. R., Büchner, T., Dombret, H., Ebert, B. L., Fenaux, P., Larson, R. A., et al. (2017). Diagnosis and management of AML in adults: 2017 ELN recommendations from an international expert panel. Blood. 129, 424-447.

In another embodiment, said medicament can be used for treating a CD123-expressing cell-mediated pathological condition or a condition characterized by the direct or indirect activity of a CD123-expressing cell. In other words, the invention is related to an anti-CD123 CAR expressing T cell comprising 80% to 100% of SEQ ID NO: 19 for its use as a medicament to treat a condition linked to the detrimental activity of CD123-expressing cells, in particular to treat a condition selected from AML, any one of the AML with adverse cytogenetic risk: t(8;21)(q22;q22.1); RUNX1-RUNX1T1, inv(16)(p13.1q22) or t(16;16)(p13.1;q22); CBFB-MYH11, Mutated NPM1 without FLT3-ITD or with FLT3-ITD^{low}, Biallelic mutated CEBPA, Mutated NPM1 and FLT3-ITD^{high}, Wild-type NPM1 without FLT3-ITD or with FLT3-ITD^{low}, t(9;11)(p21.3;q23.3); MLLT3-KMT2A, t(6;9)(p23;q34.1); DEK-NUP214, t(v;11q23.3); KMT2A rearranged, t(9;22)(q34.1;q11.2); BCR-ABL1, inv(3)(q21.3q26.2) or t(3;3)(q21.3;q26.2); GATA2, MECOM(EVI1), Wild-type NPM1 and FLT3-ITD^{high} Mutated RUNX1, Mutated ASXL1, Mutated TP53, Complex karyotype comprising three or more unrelated chromosome abnormalities in the absence of one of the World Health Organization-designated recurring translocations or inversions., t(8;21), inv(16) or t(16;16), t(9;11), t(v;11)(v;q23.3); AML with BCR-ABL1); or Monosomal karyotype comprising one single monosomy (excluding loss of X or Y) in association with at least one additional monosomy or structural chromosome abnormality (excluding core-binding factor AML); or a Mutated TP53 with Variant Allele Frequency (VAF) >10%.

In another aspect, the present invention relies on methods for treating patients in need thereof, said method comprising at least one of the following steps:
(a) providing an immune-cell UCART 123 and/or UCART CLL1
(b) Administrating said transformed immune cells to said patient, after a lymphodepletion regiment and optionally after a debulking treatment and a lymphodepleting treatment.

In one embodiment, said T cells of the invention can undergo robust in vivo T cell expansion and can persist for an extended amount of time such as 1 week, 2 weeks, 3 weeks, 1 month, two months up to 12 months.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

Cells that can be used with the disclosed methods are described in the previous section. Said treatment can be used to treat patients diagnosed wherein a pre-malignant or malignant cancer condition characterized by CD123-expressing cells or CLL1-expressing cells, especially by an overabundance of CD123-expressing cells. Such conditions are found in hematologic cancers, such as AML

Subtypes of AML also include, hairy cell leukemia, philadelphia chromosome-positive acute lymphoblastic leukemia.

AML may be classified as AML with specific genetic abnormalities. Classification is based on the ability of karyotype to predict response to induction therapy, relapse risk, survival.

Accordingly, AML that may be treated using the anti-CD123 and/or anti-CLL1 CAR-expressing cells of the present invention may be AML with a translocation between chromosomes 8 and 21, AML with a translocation or inversion in chromosome 16, AML with a translocation between chromosomes 9 and 11, APL (M3) with a translocation between chromosomes 15 and 17, AML with a translocation between chromosomes 6 and 9, AML with a translocation or inversion in chromosome 3, AML (megakaryoblastic) with a translocation between chromosomes 1 and 22.

The present invention is particularly useful for the treatment of AML associated with these particular cytogenetic markers.

The present invention also provides an anti-CD123 and/or anti-CLL1 CAR expressing T cell for the treatment of patients with specific cytogenetic subsets of AML, such as patients with t(15;17)(q22;q21) identified using all-trans retinoic acid (ATRA)16-19 and for the treatment of patients with t(8;21)(q22;q22) or inv(16)(p13q22)/t(16;16)(p13;q22) identified using repetitive doses of high-dose cytarabine.

Preferably, the present invention provides an anti-CD123 and/or anti-CLL1 CAR expressing T cell for the treatment of patients with aberrations, such as -5/del(5q), -7, abnormalities of 3q, or a complex karyotype, who have been shown to have inferior complete remission rates and survival.

Group of Patients

The invention provides a treatment for newly diagnosed patients with CD123 and/or CLL1 positive adverse genetic risk acute myeloid leukaemia (AML), and whose bone marrow blast content is <20%.

Bone marrow blast content may be <20% after 1 or 2 courses of standard intensive induction chemotherapy or after any debulking therapy to lower blasts in the bone marrow to less than 20%. This is including patients with CD123 and/or CLL1 positive AML secondary to MDS, who do not achieve morphologic or cytogenetic complete remission, and whose bone marrow blast content is <20% after 1 or 2 courses of standard intensive induction chemotherapy. AML with Adverse genetic risk is defined as defined per the ELN guidelines.

As example of debulking treatment patients may have received either a “3+7” regimen (consisting of 3 days of an IV anthracycline: daunorubicin at least 60 mg/m²; idarubicin 12 mg/m²; or mitoxantrone 12 mg/m², and 7 days of continuous infusion cytarabine (100-200 mg/m²)); or a FLAG-Ida regimen (for example consisting of fludarabine 30 mg/m² from Day 2 to Day 6, cytarabine 1500-2000 mg/m² IV, Day 2 to Day 6; idarubicin 10 mg/m², Day 2 to Day 4); and the level of blast in bone marrow must reach less than 20%

In still another embodiment, the present invention is used as a treatment in AML patients with low, poor or unfavorable status that is to say with a predicted survival of less than 20% at 5 years survival rate. In this group, patients suffering AML with the following cytogenetic characteristics Inv(3)(q21.3q26.2) or t(3;3)(q21.3;q26.2); GATA2, MECOM(EVI1); t(8;21), inv(16) or t(16;16), t(9;11), t(v;11)(v;q23.3); AML with BCR-ABL1); Monosomal karyotype (presence of one single monosomy (excluding loss of X or Y) in association with at least one additional monosomy or structural chromosome abnormality (excluding core-binding factor AML); or Mutated TP53 with variant allele frequency (VAF) >10%,

are especially contemplated to be treated according to the present invention or with an object of the present invention, provided that the level of blast in bone marrow is less than 20%.

Composition Comprising an Engineered T Cells According to the Invention for Use as a Medicament and Method Using the Same

The present invention also provides a composition for its use as a medicament or a method for treating AML with Adverse genetic risk.

The present invention also provides a composition for its use or a method for inhibiting the proliferation or reducing a CD123-expressing and/or CLL1-expressing cell population or activity in a patient with AML with Adverse genetic risk. An exemplary method includes administering a lymphodepleting treatment followed by contacting a CD123-expressing and/or CLL1-expressing AML cell with a CD 123 CART and/or CLL1 CART cell of the invention that binds to the CD123-expressing and/or CLL1-expressing AML with adverse genetic risk cells.

In a more specific aspect, the present invention provides a composition for its use or a method for inhibiting the proliferation or reducing the population of cancer cells expressing CD 123 and/or CLL1 in a patient, the methods comprising contacting the CD123-expressing and/or CLL1-expressing cancer cell population with a CD 123 CART and/or CLL1 CART cell of the invention that binds to the CD123-expressing cell, binding of a CD 123 CART cell of the invention to the CD123-expressing cancer cell resulting in the destruction of the CD123-expressing and/or CLL1-expressing cancer cells.

In certain aspects, the CD 123 CART and/or CLL1 CART cell of the invention reduces the quantity, number, amount or percentage of cells and/or cancer cells by at least 25%, at least 30%, at least 40%, at least 50%, at least 65%, at least 75%, at least 85%, at least 95%, or at least 99% (to undetectable level) in a subject with or animal model for myeloid leukemia or another cancer associated with CD123-expressing and/or CLL1-expressing cells, relative to a negative control.

The present invention also provides a composition for its use or a method for preventing, treating and/or managing a disease associated with CD123-expressing and/or CLL1-expressing cells, the method comprising administering to a subject in need a CD 123 and/or CLL1 CART cell of the invention that binds to the CD123-expressing and/or CLL1-expressing cell. In one aspect, the subject is a human.

The present invention provides a composition for its use or a method for treating or preventing AML with adverse genetic risk cells associated with CD123-expressing and/or CLL1-expressing cells, the method comprising administering to a subject in need thereof a CD 123 and/or CLL1 CAR CART cell of the invention that binds to the CD 123-expressing cell and/or CLL1-expressing. In another aspect, the methods comprise administering to the subject in need thereof an effective amount of a CD 123 CART and/or CLL1 CART cell of the invention two times in combination with an effective amount of another therapy.

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

Preferred one or more therapies against cancer comprises a debulking therapy, such as a “3+7” regimen (consisting of 3 days of an IV anthracycline: daunorubicin at least 60 mg/m²; idarubicin 12 mg/m²; or mitoxantrone 12 mg/m², and 7 days of continuous infusion cytarabine (100-200 mg/m²)); or a FLAG-Ida regimen (for example consisting of fludarabine 30 mg/m² from Day 2 to Day 6, cytarabine 1500-2000 mg/m² IV, Day 2 to Day 6; idarubicin 10 mg/m², Day 2 to Day 4);

The treatment with the anti-CD123 and/or anti-CLL1 engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy. Preferred one or more therapies against cancer comprises a debulking therapy, such as a “3+7” regimen (consisting of 3 days of an IV anthracycline: daunorubicin at least 60 mg/m²; idarubicin 12 mg/m²; or mitoxantrone 12 mg/m², and 7 days of continuous infusion cytarabine (100-200 mg/m²)); or a FLAG-Ida regimen (for example consisting of fludarabine 30 mg/m² from Day 2 to Day 6, cytarabine 1500-2000 mg/m² IV, Day 2 to Day 6; idarubicin 10 mg/m², Day 2 to Day 4);

According to a preferred embodiment of the invention, said treatment with engineered UCART123 and/or UCART CLL1 can be administrated into patients undergoing an immunosuppressive treatment. In this aspect, the immunosuppressive treatment should help the selection and expansion of the T-cells according to the invention within the patient.

Preferably, the treatment with the engineered anti-CD123 and/or anti-CLL1 CAR immune cells according to the invention may be administered in combination (e.g., simultaneously or following) with one or more lymphodepleting therapy.

In a preferred embodiment, the lymphodepleting regimen preceding UCART administration consists of fludarabine 30 mg/m²/day IV for 4 days over 15 to 30 minutes from Day −5 to Day −2 with a maximum daily dose of 60 mg, and cyclophosphamide 1 g/m²/day IV over 1 hour for 3 days from Day 4 to Day −2 with a maximum daily dose of 2 grams.

In a preferred embodiment, the lymphodepleting regimen preceding UCART123 administration consists of fludarabine 30 mg/m²/day IV for 4 days over 15 to 30 minutes from Day −5 to Day −2 with a maximum daily dose of 60 mg, and cyclophosphamide 1 g/m²/day IV over 1 hour for 3 days from Day 4 to Day −2 with a maximum daily dose of 2 grams.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermaly, intratumorally, intranodally, intramedullary, intramuscularly, by intravenous or intralymphatic injection, or intraperitoneally.

In one embodiment, the compositions of the present invention are preferably administered by intravenous injection.

The administration of the cells or population of cells of the composition of the invention can consist of the administration of 10⁴-10⁹ cells per kg body weight, preferably 10⁵ to 10⁶ cells/kg body weight including all integer values of cell numbers within those ranges.

In a preferred embodiment the cells or population of cells of the composition of the invention are administered at a dose (Dose/kg up to 80 kg-equivalent) of 2.5×10⁵ or 6.25×10⁵ or 5.05×10⁶ preferably, 5.05×10⁶/kg 4.0×10⁸ or CAR+_TCRαβ⁻_T-cells/80 kg.

TABLE 11
UCART dosages

| | UCART123 and/or | Maximum UCART123 and/or |
|---|---|---|
| | UCART CLL1 (Dose/ | UCART CLL1 Dose/Patient |
| | kg up to 80 kg- | (Based on a Patient Weight |
| Dose-level | equivalent) | equivalent of 80 kg) |
| −1 | 2.5 × 10⁵ | 2.0 × 10⁷ CAR⁺_TCRαβ⁻_T-cells |
| 1 | 6.25 × 10⁵ | |
| 2 | 5.05 × 10⁶ | 4.0 × 10⁸ CAR⁺_TCRαβ⁻_T-cells |

Theses doses correspond to a Maximum UCART 123 and/or UCART CLL1 Dose/Patient (Based on a Patient Weight equivalent of 80 kg) of 2.0×10⁷ CAR⁺_TCRαβ⁻_T-cells, 5.0×10⁷ CAR⁺_TCRαβ⁻_T-cells and 4.0×10⁸ CAR⁺_TCRαβ⁻_T-cells respectively and should be administered at the dose level of −1, 1 and 2 of the escalation dose.

Thus, the cells or population of UCART123 and/or UCART CLL1 of the invention can be administrated in one or more doses. In another embodiment, said effective amount of cells are administrated as a single dose and is enough for Complete remission CR with minimal residual disease (MRD)<0.01% (by flow cytometry or molecular methods) with no Dose Limiting Toxicity DLT.

In that case HSCT is performed.

If CR with MRD <0.01% is not reached, the effect of UCART123 and/or UCART CLL1 is partial and no toxicity is measured, the patient may benefit a second lymphodepletion followed by a second dose of UCART

Thus, the cells or population of UCART123 and/or UCART CLL1 of the invention can be administrated in one or more doses. In another embodiment, said effective amount of cells are administrated as a single dose and is enough for Complete remission CR with minimal residual disease (MRD)<0.01% (by flow cytometry or molecular methods) with no Dose Limiting Toxicity DLT.

In that case HSCT is performed 28-32 days after UCART administration.

If CR with MRD <0.01% is not reached, the effect of UCART is partial and no toxicity is measured, the patient may benefit a second lymphodepletion followed by a second dose of UCART 28-32 days after the first UCART 123 administration.

In another embodiment, said effective amount of cells are administrated as more than one dose over a period time. Timing of administration is within the judgment of managing physician and depends on the clinical condition of the patient.

The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. In a particular embodiment, UCART CD123 and/or UCART CLL1 are administered followed by Rituxan, or rituximab as agents that react with CD20.

The composition according to the invention comprising rituximab, preferably at a dose of 375 mg/m² weekly and more preferably at a dose of 375 mg/m² weekly for up to 4 weeks.

In certain embodiments of the present invention, anti-CD123 CAR and/or anti-CLL1 CAR expressing cells are administered to a patient in conjunction (e.g., before, simultaneously or following) with a drug selected from Aracytine, Cytosine Arabinoside, amsacrine, Daunorubicine, Idarubicine, Novantrone, Mitoxantrone, Vepeside, Etoposide (VP16), arsenic trioxyde, transretinoic acid, mechlorethamine, procarbazine, chlorambucil, and combination thereof. In these embodiments anti-CD123 and/or anti CLL1CAR expressing cells may be resistant to the particular drug or combination of drugs that is (are) administered in conjunction with anti-CD123 CAR expressing cells.

In other embodiments of the present invention, anti-CD123 CAR expressing cells are administered to a patient in conjunction with a drug selected from cytarabine, anthracyclines, 6-thioguanine, hydroxyurea, prednisone, and combination thereof.

Bone Marrow Transplantation

The present invention, although it may not be always necessary to cure the patients, is generally performed in view of proceeding to an autologous or allogeneic hematopoietic stem cells transplant (HSCT), thereby achieving complete and durable remission.

This optional treatment step is performed according to standard protocols and good medical practices. It usually requires treating the patient, previously treated with the engineered immune cells, with a pre-conditioning regimen prior to bone marrow transplant. Such pre-conditioning regimen are well established in the art [Peccatori, J., and Ciceri, F. (2010) Allogeneic stem cell transplantation for acute myeloid leukemia. Haematologica, 95(6), 857-859].

In a specific embodiment, the bone marrow transplantation is an allogeneic HSCT. In a specific embodiment, the allogeneic HSCT is a peripheral blood stem cell transplant. In a more specific embodiment, the population of allogeneic cells is derived from a third-party donor that preferably matches the donor of the engineered immune cells previously used.

In a specific embodiment, the population of allogeneic cells that is administered to the human patient is restricted by an HLA allele shared with the human patient.

In specific embodiments, the population of allogeneic cells comprising WT1-specific allogeneic T cells shares at least 2 out of 8 HLA alleles (for example, two HLA-A alleles, two HLA-B alleles, two HLA-C alleles, and two HLA-DR alleles) with the human patient.

By “Matching” is meant that, preferably, at least 10 HLA markers (alleles) out of 10 (10/10) of UCART cells are matching with HSCT: two A markers, two B markers, two C markers, two DRB1 markers and two DQ, to match, when UCART cells are not from said patient less matching may be permitted with the limit of mismatching being set as 2 mismatching out of 8, or 3 out of 8 and some mismatching to be avoided to be defined as below.

For example, an adult donor should generally match at least 6 of the 8 HLA markers (two A markers, two B markers, two C markers, two DRB1 markers), Preferably, at least a 7 of 8 match. If using cord blood cells as original material, a cord blood unit should generally match at least 4 of 6 ( 4/6) again no more than 2 mismatches) markers at HLA-A, —B, and -DRB1

In the preferred case of matching donors, cells may be from matching twins, siblings, donors with from most preferred to less preferred 10/10, 9/9, 8/8, 7/7, 6/6 HLA matching or any of such cells engineered to match HSCT.

According to a preferred embodiment the HSCs used for the bone marrow transplant are HLA matching the engineered immune cells and preferably originate from the same donor. In particular the methods of the invention proceed with obtaining immune cells, such as T-cells and HSC from the same donor, separately engineering the immune cells in view of performing allogeneic CAR or modified TCR T-cell therapy and the HSCs in view of performing a following-up bone marrow transplantation.

According to a specific aspect of the invention the HSCs may be gene edited to improve HLA matching, such as to obtain gene replacement of HLA alleles.

Methods for HLA testing have dramatically improved over the past 20 years, and today patients receiving a well matched unrelated donor in experienced transplant centers have similar outcome to HLA-identical sibling recipients. Furthermore, the organization of hematopoietic stem cell donor registries has improved dramatically in recent years, resulting in a successful recruitment of a matched donor in 50-80% of patients in an appropriate time according to disease status. However, patients from ethnicities less represented in world-wide registries still have a significantly lower chance of finding a well matched donor. In recent years, a third source of stem cells, umbilical cord blood, has become more and more popular. Cord blood has several potential advantages, including rapid availability and lower risk of graft versus host disease, resulting in less stringent HLA-matching requirements. Nowadays in the US, umbilical cord blood transplants represent around a third of all transplants for children with acute leukemia; the use of umbilical cord blood is also increasing in adults, particularly following the advent of double-unit transplants to augment graft cell dose [Delaney C, et al. Cord blood transplantation for haematological malignancies: conditioning regimens, double cord transplant and infectious complications. Br J Haematol. 2009; 147(2):207-16]. Aversa F, et al. [Full haplotype-mismatched hematopoietic stem-cell transplantation: a phase II study in patients with acute leukemia at high risk of relapse. J Clin Oncol. 2005; 23(15):3447-54] carried out seminal work on profound T-cell depletion in the setting of HLA-haploidentical hematopoietic stem cell transplantation associated to infusion of large numbers of purified CD34⁺ cells resulting in high engraftment rate and low incidence of graft versus host disease. Since then, many achievements have been made in this setting. Different strategies to speed up the immunoreconstitution have been developed, such as the infusion of genetically modified lymphocytes post-transplant [Ciceri F, et al. Infusion of suicide-gene-engineered donor lymphocytes after family haploidentical haemopoietic stem-cell transplantation for leukaemia (the TK007 trial): a non-randomised phase I-II study. Lancet Oncol. 2009; 10(5):489-500] or different strategies of in vivo T-cell depletion i.e. CD3/CD19 negative selection [Bethge W A, et al. Haploidentical allogeneic hematopoietic cell transplantation in adults using CD3/CD19 depletion and reduced intensity conditioning: an update. Blood Cells Mol Dis. 2008; 40(1):13-9] In the last few years, the infusion of un-manipulated haploidentical stem cells has also been investigated, using alternative strategies of post-transplant immunosuppression; among these the administration of rapamycin to promote in vivo T-regulatory cell expansion or the use of cyclophosphamide on day 3 after graft infusion to reduce alloreactive lymphocytes. All these advances in the field of alternative donors and multiple options in stem cell sources and content are now expected to translate into a higher rate of patients undergoing hematopoietic stem cell transplantation according to the present invention.

Regarding the pre-conditioning step, the concomitant use of alemtuzumab and cyclosporine A exposure in the first post-transplant days appears to be a potentially valuable strategy to improve the outcome of very high-risk patients, for example those with adverse cytogenetics at diagnosis. Manipulation of immunosuppressive therapy post-transplant should be performed not only according to patient characteristics, but also considering graft source and quantity of donor T cells infused, modality of T depletion (alemtuzumab, anti-lymphocyte globulins-ATG or others) and HLA matching. High resolution matching of HLA-A, -B, -C, -DRB1 and -DQB1 (10/10) can improve clinical outcome in terms of overall survival, transplant related mortality and acute graft versus host disease; but it is now emerging that also matching at HLA-DPB1 can be important. HLA-DPB1 displays weak linkage disequilibrium with the other class II loci; therefore, only approximately 15% of 10/10 matched pairs are also matched for HLA-DPB1 (12/12). HLA-DPB1 allele-mismatched transplantations permissive according to a new functional algorithm developed by Fleischhauer et al. have better outcome in terms of survival [Crocchiolo R, et al. Nonpermissive HLA-DPB1 disparity is a significant independent risk factor for mortality after unrelated hematopoietic stem cell transplantation. Blood. 2009; 114(7):1437-44] Besides tuning cyclosporine A exposure, new immunosuppressive strategies are becoming available, above all the use of rapamycin as graft versus host disease prophylaxis. Rapamycin is an immunosuppressive drug that arrests cell cycle in G1 through the inhibition of DNA transcription, DNA translation and protein synthesis but, in contrast to calcineurin inhibitors, promotes the generation of T-regulatory cells (Tregs). Besides its intriguing effect on Tregs, rapamycin has also a potential antitumor activity in different hematologic malignancies, rendering it suitable for high-risk patients.

### Other Definitions

Unless otherwise specified, “a,” “an,” “the,” and “at least one” are used interchangeably and mean one or more than one.-Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: an is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
The term “endonuclease” or TAL-endonuclease refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 12 base pairs (bp) in length, more preferably of 14-55 bp. Rare-cutting endonucleases significantly increase HR by inducing DNA double-strand breaks (DSBs) at a defined locus (Perrin, Buckle et al. 1993; Rouet, Smih et al. 1994; Choulika, Perrin et al. 1995; Pingoud and Silva 2007). Rare-cutting endonucleases can for example be a homing endonuclease (Paques and Duchateau 2007), a chimeric Zinc-Finger nuclease (ZFN) resulting from the fusion of engineered zinc-finger domains with the catalytic domain of a restriction enzyme such as FokI (Porteus and Carroll 2005), a Cas9 endonuclease from CRISPR system (Gasiunas, Barrangou et al. 2012; Jinek, Chylinski et al. 2012; Cong, Ran et al. 2013; Mali, Yang et al. 2013) or a chemical endonuclease (Eisenschmidt, Lanio et al. 2005; Arimondo, Thomas et al. 2006). In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer 2005). Such chemical endonucleases are comprised in the term “endonuclease” according to the present invention.
By a “TALE-nuclease” (TALEN® is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-1. In a particular embodiment, the TALE domain can be fused to a meganuclease like for instance I-CreI and 1-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of 1-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALE-nuclease have been already described and used to stimulate gene targeting and gene modifications [Christian, Cermak et al. (2010) Targeting DNA Double-Strand Breaks with TAL Effector Nucleases. Genetics. 186(2):757-761]. Engineered TAL-nucleases are commercially available under the trade name TALEN® (Cellectis, 8 rue de la Croix Jerry, 75013 Paris, France).
By “delivery vector” or “delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adenoassociated viruses AAV6), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lenti-virus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as nonlimiting example, that are able to integrate the genome of a target cell. At the opposite by “non-integrative lentiviral vectors (or NILV)” is meant efficient gene delivery vectors that do not integrate the genome of a target cell through the action of the virus integrase.

By adeno associated vector is meant AAV6 particles comprising AAV2 Inverted terminal repeats and a gene to be inserted into the genome. These particles are used with TAL-proteins (TALEN®, in particular TALEN® targeting the TCR alpha gene, CD25 gene, beta2 microglobulin gene as described in PCT/EP2017/076798.
Delivery vectors and vectors can be associated or combined with any cellular permeabilization techniques such as sonoporation or electroporation or derivatives of these techniques.
By cell or cells is intended any eukaryotic living cells, primary cells and cell lines derived from these organisms for in vitro cultures.
By “primary cell” or “primary cells” are intended cells taken from living tissue (i.e. biopsy material) and established for growth in vitro for a limited amount of time by contrast to continuous cell lines (e.g. tumorigenic or artificially immortalized cell lines). Non-limiting examples of continuous cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells. In general, primary immune cells are provided from a donor or a patient through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284). The primary immune cells according to the present invention can also be differentiated from stem cells, such as cord blood stem cells, progenitor cells, bone marrow stem cells, hematopoietic stem cells (HSC) and induced pluripotent stem cells (iPS).
by “mutation” is intended the substitution, deletion, insertion of up to one, two, three, four, five, six, seven, eight, nine, ten, eleven, twelve, thirteen, fourteen, fifteen, twenty, twenty five, thirty, fourty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “variant(s)”, it is intended a repeat variant, a variant, a DNA binding variant, a TALE-nuclease variant, a polypeptide variant obtained by mutation or replacement of at least one residue in the amino acid sequence of the parent molecule.
by “functional variant” is intended a catalytically active mutant of a protein or a protein domain; such mutant may have the same activity compared to its parent protein or protein domain or additional properties, or higher or lower activity.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means.
“signal-transducing domain” or “co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

A “co-stimulatory molecule” refers to the cognate binding partner on a T cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor.

A “co-stimulatory signal” as used herein refers to a signal, which in combination with primary signal, such as TCR/CD3 ligation, leads to T cell proliferation and/or upregulation or downregulation of key molecules.

The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. Thus examples of cell surface markers that may act as ligands include those associated with viral, bacterial and parasitic infections, autoimmune disease and cancer cells.

The term “subject” or “patient” as used herein includes all members of the animal kingdom including non-human primates and humans, preferably primates, more preferably a human.

Newly diagnosed patients may be part of the present invention especially when their bone marrow has less 20% blasts.

The term “relapsed” refers to a situation where a subject or a mammal, who has had a remission of cancer after therapy has a return of cancer cells.

The term “refractory or resistant” refers to a circumstance where a subject or a mammal, even after intensive treatment, has residual cancer cells in his body.

The term “drug resistance” refers to the condition when a disease does not respond to the treatment of a drug or drugs. Drug resistance can be either intrinsic (or primary resistance), which means the disease has never been responsive to the drug or drugs, or it can be acquired, which means the disease ceases responding to a drug or drugs that the disease had previously responded to (secondary resistance). In certain embodiments, drug resistance is intrinsic. In certain embodiments, the drug resistance is acquired.

The term “hematologic malignancy” or “hematologic cancer” refers to a cancer of the body's blood-bone marrow and/or lymphatic tissue. Examples of hematological malignancies include, for instance, myelodysplasia, leukemia, lymphomas, such as cutaneous Lymphomas, non-Hodgkin's lymphoma, Hodgkin's disease (also called Hodgkin's lymphoma), and myeloma, such as acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM). In a preferred embodiment hematologic malignancy” or “hematologic cancer” refers to AML with adverse genetic risk and patients having less than 20% blasts in bone marrow after no treatment 1 or 2 debulking or first line treatment.

The term “leukemia” refers to malignant neoplasms of the blood-forming tissues, including, but not limited to, chronic lymphocytic leukemia or chronic lymphoid leukemia, chronic myelocytic leukemia, or chronic myelogenous leukemia, acute lymphoblastic leukemia, acute myeloid leukemia or acute myelogenous leukemia (AML) also acute myeloblastic leukemia.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified. All objects described below may be an object of the present invention.

The present invention non imitatively encompasses the following aspects:
1. A composition comprising i) at least one or two immunotherapy composition (s) consisting of a lymphodepleting treatment and a dose of engineered immune cells expressing at the cell surface membrane, a chimeric antigen receptor (CAR) specific for a tumoral antigen (autologous or allogenic CAR+_T-cells, preferably anti-CD123 CAR+_T-cells), for treating a patient suffering an haematological cancer, preferably acute myeloid leukaemia (AML), AML with adverse genetic risk (or adverse cytogenetic risk), AML with adverse genetic risk and with less than 20% blasts in the bone marrow, optionally ii) one or two debulking treatment(s), or
A composition comprising i) at least one or two immunotherapy composition (s) consisting of a lymphodepleting treatment and a dose of engineered immune cells expressing at the cell surface membrane, a chimeric antigen receptor (CAR) specific for a tumoral antigen (autologous or allogenic CAR+_T-cells, preferably anti-CD123 CAR+_T-cells), for treating a patient suffering AML with adverse genetic risk and with less than 20% blasts in the bone marrow, optionally ii) one or two debulking treatment(s), or
A composition comprising i) at least one or two immunotherapy composition (s) consisting of a lymphodepleting treatment and a dose of engineered immune cells expressing at the cell surface membrane, a chimeric antigen receptor (CAR) specific for a tumoral antigen (autologous or allogenic CAR+_T-cells, preferably anti-CD123 CAR+_T-cells), for treating a patient suffering an haematological cancer, preferably acute myeloid leukaemia (AML), AML with adverse genetic risk (or adverse cytogenetic risk), optionally ii) one or two debulking treatment(s).
2. The composition of item 1 wherein engineered immune cells comprise CAR+_TCRαβ− T-cells, preferably anti-CD123 CAR+_TCRαβ—_T-cells, anti-CLL-1 CAR+_TCRαβ−_T-cells, anti-CD123 anti-CLL-1-CAR+_TCRαβ−_T-cells.
3. The composition according to item 1 or 2 wherein said patient is suffering AML with an adverse genetic risk selected from: t(8;21)(q22;q22.1); RUNX1-RUNX1T1, inv(16)(p13.1q22) or t(16;16)(p13.1;q22); CBFB-MYH11, Mutated NPM1 without FLT3-ITD or with FLT3-ITDlow, Biallelic mutated CEBPA, Mutated NPM1 and FLT3-ITDhigh, Wild-type NPM1 without FLT3-ITD or with FLT3-ITDlow, t(9;11)(p21.3;q23.3); MLLT3-KMT2A, t(6;9)(p23;q34.1); DEK-NUP214, t(v;11q23.3); KMT2A rearranged, t(9;22)(q34.1;q11.2); BCR-ABL1, inv(3)(q21.3q26.2) or t(3;3)(q21.3;q26.2); GATA2, MECOM(EVI1), Wild-type NPM1 and FLT3-ITDhigh Mutated RUNX1, Mutated ASXL1, Mutated TP53, Complex karyotype comprising three or more unrelated chromosome abnormalities in the absence of one of the World Health Organization-designated recurring translocations or inversions., t(8;21), inv(16) or t(16;16), t(9;11), t(v;11)(v;q23.3); AML with BCR-ABL1); or Monosomal karyotype comprising one single monosomy (excluding loss of X or Y) in association with at least one additional monosomy or structural chromosome abnormality (excluding core-binding factor AML); or a Mutated TP53 with Variant Allele Frequency (VAF) >10%, preferably an AML with adverse genetic risk selected from Inv(3)(q21.3q26.2), t(3;3)(q21.3;q26.2); GATA2, MECOM(EVI1); a Complex karyotype comprising Three or more unrelated chromosome abnormalities in the absence of one of the World Health Organization-designated recurring translocations or inversions, such as., t(8;21), inv(16), t(16;16), t(9;11), t(v;11)(v;q23.3); AML with BCR-ABL1); a Monosomal karyotype comprising one single monosomy (excluding loss of X or Y) in association with at least one additional monosomy or structural chromosome abnormality (excluding core-binding factor AML); a Mutated TP53 with Variant Allele Frequency (VAF) >10%.
4. The composition according to any one of items 1 to 3 wherein engineered immune cells express at the cell surface membrane, a chimeric antigen receptor (CAR) specific for a tumoral antigen selected from CD25, CD30, CD37, CD38, CD33, CD47, CD98, CD123, FLT3, CLL-1, CD56, CD117, CD133, CD157, c-kit, CD34, MUC1, CXCR4, VEGF, NKG2D_F, folate receptor beta (FR beta), hepatocyte growth factor (HGF), HLA-A2, human C-type lectin-like molecule-1 (CLL1), Lewis Y, a combination thereof, preferably specific for CD123 and/or CLL-1.
5. The composition for treating a patient according to any one of item 1 to 4 wherein a lymphodepleting treatment or regiment comprises fludarabine and Cyclophosphamide, preferably fludarabine at a dose of 30 mg/m²/day from Day −5 to Day −2 with a maximum daily dose of 60 mg; and Cyclophosphamide 1 g/m²/day from Day −4 to Day −2 with a maximum daily dose of 2 grams.
6. The composition for treating a patient according to any one of item 1 to 5 wherein said patient has less than 20% blasts in the bone marrow after 0, 1 or 2 courses of a debulking treatment such as standard intensive induction chemotherapy.
7. The composition according to any one of item 1 to 6 wherein the debulking treatment is selected from a “3+7” regimen and a FLAG-Ida regimen.
8. The composition according to any one of item 1 to 6 wherein said FLAG-Ida regimen comprises fludarabine 30 mg/m² from Day 2 to Day 6, cytarabine 1500-2000 mg/m² IV, Day 2 to Day 6; idarubicin 10 mg/m², Day 2 to Day 4).
9. The composition according to any one of item 1 to 6 wherein said 3+7″ regimen comprises 3 days of an IV anthracycline: daunorubicin at least 60 mg/m²; idarubicin 12 mg/m²; or mitoxantrone 12 mg/m², and 7 days of continuous infusion cytarabine (100-200 mg/m²).
10. The composition according to any one of item 1 to 9 comprising at least two doses of engineered immune cells expressing at the cell surface membrane, a chimeric antigen receptor (UCART) specific for a tumoral antigen, each of the at least two doses being administered after a lymphodepletion treatment, provided that: i) below of basal toxicity (grade 1) was measured after administration of the first dose (from the day of administration of the first dose to at least 7 days after, to at least 14 days after to to at least 28 days after administration of the first dose) ii) the first dose was active and iii) the two doses are the same dose or the second dose is 2 times higher than the first dose, preferably comprised between 104 to 109 UCART cells/kg and more preferably 5.05×106 cells/kg.
11. The composition according to item 10 for patients who did not achieve a morphological Complete Remission with negative Minimal Residual Disease (MRD) (defined as MRD <0.01% by flow cytometry or molecular methods) after the first UCART dose administration, and provided that no Dose Limiting Toxicity (DLT) has been observed after the first UCART dose administration.
12. The composition of any one of item 1 to 11 further comprising haematopoietic stem cells for transplantation (HSCT), optionally HSC are HLA matching the UCART.
13. The composition of any one of item 1 to 12 wherein the lymphodepletion is administered at least 3 days, preferably 5 days before administration of the UCART.
14. The composition of any one of item 1 to 13 wherein the tumoral antigen is CD123 and expressed in Universal (MHC-class I—TCRαβ−_T cells).
15. The composition according to any one of item 1 to 14 comprising Rituximab, preferably a dose of rituximab to eliminate UCART cells through binding to co-expressed RQR8 or (R)n with n is 1 to 3.
16. The composition according to item 15 comprising rituximab, preferably at a dose of 375 mg/m2 weekly and more preferably at a dose of 375 mg/m2 weekly for up to 4 weeks.
17. The composition according to any one of item 1 or 16 wherein engineered immune cells comprise less than 3% TCR-positive cells as determined by flow cytometry analysis using an anti-alpha beta TCR antibody.
18. The composition according to any one of item 1 to 17 wherein engineered immune cells comprise more than 40% and up to 99% TCRalphabeta negative and CD52 negative cells, or more than 40% and up to 99% of TCRalphabeta negative and beta2microglobulin negative cells or more than 40% and up to 88% TCRalpha beta negative, CD52 negative, beta2 microglobulin negative cells.
19. The composition according to any one of item 1 to 18 wherein engineered immune cells comprise more than 40% and up to 99% CAR+/HLAE+ cells.
20. The composition according to any one of item 1 to 19 wherein engineered immune cells are engineered using specific TALEN®.
21. The composition according to any one of item 1 to 20 wherein engineered immune cells are engineered using specific TALEN® and comprise at least one of the following DNA modifications: an exogenous DNA sequence encoding a CAR inserted into the genome, an exogenous sequence encoding an HLA-E-peptide fusion peptide inserted into the B2M gene, an alpha TCR KO gene, A B2M KO gene, a combination thereof.
22. The composition according to any one of item 1 to 21 wherein the engineered immune cell is an engineered immune T cell, preferably an engineered primary T cell derived from T-lymphocytes or from a human stem cell.
23. The composition according to any one of item 1 to 22 wherein the second dose of engineered immune cells is from 2.5×104 cells/kg, to 5.05×108 cells/kg, preferably 2.5×105 cells/kg, 6.25×105 cells/kg, or 5.05×106 cells/kg and the same as the first dose or higher, from 1.5 to 100 times higher.
24. The composition according to any one of item 1 to 23 for the treatment of a hematological cancer in a patient comprising:
a) Identifying a patient with hematological cancer such as leukemia, preferably AML, more preferably AML with adverse cytogenetic risk, even more preferably with adverse cytogenetic risk selected from the group consisting of: t(8;21)(q22;q22.1); RUNX1-RUNX1T1, inv(16)(p13.1q22) or t(16;16)(p13.1;q22); CBFB-MYH11, Mutated NPM1 without FLT3-ITD or with FLT3-ITDlow, Biallelic mutated CEBPA, Mutated NPM1 and FLT3-ITDhigh, Wild-type NPM1 without FLT3-ITD or with FLT3-ITDlow, t(9;11)(p21.3;q23.3); MLLT3-KMT2A, t(6;9)(p23;q34.1); DEK-NUP214, t(v;11q23.3); KMT2A rearranged, t(9;22)(q34.1;q11.2); BCR-ABL1, inv(3)(q21.3q26.2) or t(3;3)(q21.3;q26.2); GATA2, MECOM(EVI1), Wild-type NPM1 and FLT3-ITDhigh Mutated RUNX1, Mutated ASXL1, Mutated TP53, Complex karyotype comprising three or more unrelated chromosome abnormalities in the absence of one of the World Health Organization-designated recurring translocations or inversions., t(8;21), inv(16) or t(16;16), t(9;11), t(v;11)(v;q23.3); AML with BCR-ABL1); or Monosomal karyotype comprising one single monosomy (excluding loss of X or Y) in association with at least one additional monosomy or structural chromosome abnormality (excluding core-binding factor AML); or a Mutated TP53 with Variant Allele Frequency (VAF) >10%,
b) measuring blasts content over total cells in a sample of the bone marrow of said patient, if blast content is less than 20% over total cells in the bone marrow go directly to step (d)
c) administering at least one or two debulking treatment(s) to reach less than 20% blasts in the bone marrow; and eventually minimize Cytokine releasing syndrome (CRS),
d) administering an immunotherapy composition consisting of a lymphoDepleting treatment or regimen and of at least one dose of CART (autologous transfert) or UCART (allogenic transfer), preferably targeting CD123 and/or CLL-1, for reaching complete remission (Minimal residual disease (<0.1%)),
e) Measuring blasts in the bone marrow, and
f) If complete remission (Minimal residual disease (<0.1%)) is not achieved, partial remission is measured and toxicity measured after step (d) is at basal level or of grade 1 level: administering a second immunotherapy composition consisting of a lymphodepleting treatment or regimen and a dose of engineered immune cells [CART (autologous transfer) or UCART (allogenic transfer)].
g) If complete remission is achieved (Minimal residual disease (<0.1%)), at step d) or f) transplanting bone marrow stem cells.
25. The composition according to any one of item 1 to 24 wherein the first dose of CART or of UCART is from 2.5×104/kg, to 5.05×108/kg, preferably 2.5×105/kg, 6.25×105/kg, or 5.05×106/kg.
26. The composition according to any one of item 1 to 25 wherein the second dose of CART or UCART is from 2.5×104/kg, to 5.05×108/kg, preferably 2.5×105/kg, 6.25×105/kg, or 5.05×106/kg and is the same as the first dose or to 1.5 to 100 times higher.
27. The composition according to any one of item 1 to 26 wherein the second dose of engineered cells is administered from Day 15-35, preferably day 28-35 following the first infusion of engineered cells.
28. The composition according to any one of item 1 to 27 wherein cells in the first dose of engineered cell are originally from the patient intended to be treated and engineered to express a CAR targeting the tumor (CART) or from a healthy donor (UCART expressing a CAR targeting the tumor and with at least an inactivated TCR alpha).
29. The composition according to any one of item 1 to 28 wherein cells in said second dose of engineered cell are from the same batch than cells of the first dose.
30. The composition according to any one of item 1 to 28 wherein cells in said second dose of engineered cell are from a different batch than cells of the first dose and from the same individual than cells in said first dose.
31. The composition according to any one of item 1 to 28 wherein cells in the first dose are autologous or allogenic and cells in said second dose are allogenic cells, provided that if cells in the first and second dose are allogenic, they are from the same donor and time between two injections (and two lymphodepletions) is 15-35 days, preferably day 28-35 days following the first infusion of engineered cells.
32. The composition according to any one of item 1 to 28 wherein cells in the first dose are allogenic and cells in said second dose are allogenic cells from another donor provided that MHC molecules of cells of the first dose match MHC molecules of the patient and/or have non common HLA allele with the HLA molecule of the second dose to avoid an anamnestic response.
33. The composition according to any one of item 1 to 28 wherein cells in the first dose are allogenic, cells in said second dose are allogenic cells and MHC molecules of cells of the first dose have non common HLA allele with the HLA molecule of the second dose to avoid an anamnestic response and match cells of the HSCT.
34. A method for achieving remission or even eliminating a hematological cancer in a patient comprising:
a) Identifying a patient with hematological cancer such as leukemia, preferably AML, more preferably AML with adverse cytogenetic risk, even more preferably with adverse cytogenetic risk,
b) measuring blasts content over total cells in a sample of the bone marrow of said patient, if blast content is less than 20% over total cells in the bone marrow go to step (d)
c) if blast content is more than 20% over total cells in the bone marrow: administering at least one or two debulking treatment(s) to reach less than 20% blasts in the bone marrow; and minimize CRS,
d) lymphoDepleting said patient and administering one dose of CART (autologous) or UCART (allogenic transfer).
e) Measuring blasts in the bone marrow,
f) If complete remission (Minimal residual disease <0.1%), is not achieved but partial remission is measured and no or basal level toxicity was observed after step (d) administering a second lymphoDepleting treatment and administering a second dose of engineered cell (CART or UCART),
g) If complete remission is achieved (Minimal residual disease <0.1%), transplanting bone marrow stem cells.
35. The method according to item 34 wherein the dose of engineered cells is from 2.5×104/kg, to 5.05×108/kg, preferably 2.5×105/kg, 6.25×105/kg, or 5.05×106/kg.
36. The method according to item 34 or 35 wherein the second dose of engineered cells is from 2.5×104/kg, to 5.05×108/kg, preferably 2.5×105/kg, 6.25×105/kg, or 5.05×106/kg and is the same as the first dose or 1.5 to 100 times higher.
37. The method according to item 34 to 36, wherein said UCART cell is from a healthy donor.
38. The method according to item 34 to 36, wherein said CART cell is from the patient.
39. A composition comprising:
At least one debulking treatment comprising fludarabine 30 mg/m² from Day 2 to Day 6, cytarabine 1500-2000 mg/m² IV, Day 2 to Day 6; idarubicin 10 mg/m², Day 2 to Day 4), for reducing blast content in the bone marrow to less than 20%
two treatments by immunotherapy, said treatment by immunotherapy comprising a combination of a lymphodepleting treatment and of a dose alpha beta-TCR-negative anti-CD123 CAR+_T-cells to be given successively,
said lymphodepleting treatment comprising fludarabine at a dose of 30 mg/m²/day from Day −5 to Day −2 with a maximum daily dose of 60 mg and Cyclophosphamide ate a dose of 1 g/m²/day from Day −4 to Day −2 with a maximum daily dose of 2 grams and
engineered immune cells expressing at the cell surface membrane, a chimeric antigen receptor (CAR) specific for CD123 (alpha beta-TCR-negative anti-CD123 CAR+_T-cells), being administered at a dose of a dose of 5.05×106/kg,
hematopoietic stem cells for transplantation.
   40. A composition comprising:
At least one debulking treatment comprising 3 days of an IV anthracycline: daunorubicin at least 60 mg/m²; idarubicin 12 mg/m²; or mitoxantrone 12 mg/m², and 7 days of continuous infusion cytarabine (100-200 mg/m²), for reducing blast content in the bone marrow to less than 20%
two treatments by immunotherapy, said treatment by immunotherapy comprising a combination of a lymphodepleting treatment and of a dose alpha beta-TCR-negative anti-CD123 CAR+_T-cells to be given successively,
said lymphodepleting treatment comprising fludarabine at a dose of 30 mg/m²/day from Day −5 to Day −2 with a maximum daily dose of 60 mg and Cyclophosphamide ate a dose of 1 g/m²/day from Day −4 to Day −2 with a maximum daily dose of 2 grams and
engineered immune cells expressing at the cell surface membrane, a chimeric antigen receptor (CAR) specific for CD123 (alpha beta-TCR-negative anti-CD123 CAR+_T-cells), being administered at a dose of a dose of 5.05×106/kg,
hematopoietic stem cells for transplantation.
   for treating a patient suffering AML with adverse genetic risk (or adverse cytogenetic risk).
   41. A composition comprising:
A first debulking treatment comprising 3 days of an IV anthracycline: daunorubicin at least 60 mg/m²; idarubicin 12 mg/m²; or mitoxantrone 12 mg/m², and 7 days of continuous infusion cytarabine (100-200 mg/m²), a second debulking treatment fludarabine 30 mg/m² from Day 2 to Day 6, cytarabine 1500-2000 mg/m² IV, Day 2 to Day 6; idarubicin 10 mg/m², Day 2 to Day 4), for reducing blast content in the bone marrow to less than 20%
two treatments by immunotherapy, said treatment by immunotherapy comprising a combination of a lymphodepleting treatment and of a dose alpha beta-TCR-negative anti-CD123 CAR+_T-cells to be given successively,
said lymphodepleting treatment comprising fludarabine at a dose of 30 mg/m²/day from Day −5 to Day −2 with a maximum daily dose of 60 mg and Cyclophosphamide ate a dose of 1 g/m²/day from Day −4 to Day −2 with a maximum daily dose of 2 grams and
engineered immune cells expressing at the cell surface membrane, a chimeric antigen receptor (CAR) specific for CD123 (alpha beta-TCR-negative anti-CD123 CAR+_T-cells), being administered at a dose of a dose of 5.05×106/kg,
hematopoietic stem cells for transplantation.
   for treating a patient suffering AML with adverse genetic risk (or adverse cytogenetic risk).
   42. A composition comprising:
At least one debulking treatment comprising fludarabine 30 mg/m² from Day 2 to Day 6, cytarabine 1500-2000 mg/m² IV, Day 2 to Day 6; idarubicin 10 mg/m², Day 2 to Day 4), for reducing blast content in the bone marrow to less than 20%
one treatment by immunotherapy, said treatment by immunotherapy comprising a combination of a lymphodepleting treatment and of a dose alpha beta-TCR-negative anti-CD123 CAR+_T-cells to be given successively,
said lymphodepleting treatment comprising fludarabine at a dose of 30 mg/m²/day from Day −5 to Day −2 with a maximum daily dose of 60 mg and Cyclophosphamide ate a dose of 1 g/m²/day from Day −4 to Day −2 with a maximum daily dose of 2 grams and
engineered immune cells expressing at the cell surface membrane, a chimeric antigen receptor (CAR) specific for CD123 (alpha beta-TCR-negative anti-CD123 CAR+_T-cells), being administered at a dose of a dose of 5.05×106/kg,
hematopoietic stem cells for transplantation.
   43. A composition comprising:
At least one debulking treatment comprising 3 days of an IV anthracycline: daunorubicin at least 60 mg/m²; idarubicin 12 mg/m²; or mitoxantrone 12 mg/m², and 7 days of continuous infusion cytarabine (100-200 mg/m²), for reducing blast content in the bone marrow to less than 20%
one treatments by immunotherapy, said treatment by immunotherapy comprising a combination of a lymphodepleting treatment and of a dose alpha beta-TCR-negative anti-CD123 CAR+_T-cells to be given successively,
said lymphodepleting treatment comprising fludarabine at a dose of 30 mg/m²/day from Day −5 to Day −2 with a maximum daily dose of 60 mg and Cyclophosphamide ate a dose of 1 g/m²/day from Day −4 to Day −2 with a maximum daily dose of 2 grams and
engineered immune cells expressing at the cell surface membrane, a chimeric antigen receptor (CAR) specific for CD123 (alpha beta-TCR-negative anti-CD123 CAR+_T-cells), being administered at a dose of a dose of 5.05×106/kg,
hematopoietic stem cells for transplantation.
   for treating a patient suffering AML with adverse genetic risk (or adverse cytogenetic risk).
   44. A composition comprising:
A first debulking treatment comprising 3 days of an IV anthracycline: daunorubicin at least 60 mg/m²; idarubicin 12 mg/m²; or mitoxantrone 12 mg/m², and 7 days of continuous infusion cytarabine (100-200 mg/m²), a second debulking treatment fludarabine 30 mg/m² from Day 2 to Day 6, cytarabine 1500-2000 mg/m² IV, Day 2 to Day 6; idarubicin 10 mg/m², Day 2 to Day 4), for reducing blast content in the bone marrow to less than 20%
one treatment by immunotherapy, said treatment by immunotherapy comprising a combination of a lymphodepleting treatment and of a dose alpha beta-TCR-negative anti-CD123 CAR+_T-cells to be given successively,
said lymphodepleting treatment comprising fludarabine at a dose of 30 mg/m²/day from Day −5 to Day −2 with a maximum daily dose of 60 mg and Cyclophosphamide ate a dose of 1 g/m²/day from Day −4 to Day −2 with a maximum daily dose of 2 grams and
engineered immune cells expressing at the cell surface membrane, a chimeric antigen receptor (CAR) specific for CD123 (alpha beta-TCR-negative anti-CD123 CAR+_T-cells), being administered at a dose of a dose of 5.05×106/kg,
hematopoietic stem cells for transplantation.
   for treating a patient suffering AML with adverse genetic risk (or adverse cytogenetic risk).
   45. The composition according to any one of item 39 to 44 for the treatment of AML with adverse cytogenetic risk selected from the group consisting of: t(8;21)(q22;q22.1); RUNX1-RUNX1T1, inv(16)(p13.1q22) or t(16;16)(p13.1;q22); CBFB-MYH11, Mutated NPM1 without FLT3-ITD or with FLT3-ITDlow, Biallelic mutated CEBPA, Mutated NPM1 and FLT3-ITDhigh, Wild-type NPM1 without FLT3-ITD or with FLT3-ITDlow, t(9;11)(p21.3;q23.3); MLLT3-KMT2A, t(6;9)(p23;q34.1); DEK-NUP214, t(v;11q23.3); KMT2A rearranged, t(9;22)(q34.1;q11.2); BCR-ABL1, inv(3)(q21.3q26.2) or t(3;3)(q21.3;q26.2); GATA2, MECOM(EVI1), Wild-type NPM1 and FLT3-ITDhigh Mutated RUNX1, Mutated ASXL1, Mutated TP53, Complex karyotype comprising three or more unrelated chromosome abnormalities in the absence of one of the World Health Organization-designated recurring translocations or inversions., t(8;21), inv(16) or t(16;16), t(9;11), t(v;11)(v;q23.3); AML with BCR-ABL1); or Monosomal karyotype comprising one single monosomy (excluding loss of X or Y) in association with at least one additional monosomy or structural chromosome abnormality (excluding core-binding factor AML); or a Mutated TP53 with Variant Allele Frequency (VAF) >10%,
   46. A composition according to any one of item 39 to 45, wherein engineered cells are autologous cells and TCR KO and B2M KO.
   The invention also provides a method for identifying evaluating the toxicity of an engineered cells expressing a chimeric antigen receptor comprising two alternatives descalating or escalating a dose based on the occurrence of Dose Limiting Toxicities (DLTs).
   In another embodiment, is provided a method comprising a Dose-Level “1” (DL1), in which a group of patients are to receive a dose X of engineered T cells per kilogram of body weight, a “Dose-Level “2” (DL2), in which patients are to receive about 10 times more cells than for DL1 and a Dose-Level “−1” (DL-1), in which patients receive between 2 to 5 times less cells than in DL1.
   The method as above wherein the following dose-escalation or dose de-escalation rules applied:
The dose escalation is guided by the toxicities observed, according to the modified TPI 2 design.
Optionally a greater dose increments up to 50 is tested
Patients may be included by cohorts of 2 to 4;
For each previously untested dose-level, only one patient is initially treated to check the absence of life-threatening toxicity at this dose. Optionally, after a minimum period of 2 weeks, subsequent patients were treated;
Decision to escalate or descalate to the next dose-level is based upon DLTs (Dose-Limiting Toxicity) that occurred, when the last patient of a cohort was terminated the DLT observation period.

### EXAMPLES

### Example 1: Production of TCRalpha Inactivated Cells Expressing a CD123-CAR (UCART123)

Heterodimeric TALE-nuclease targeting two 17-bp long sequences (called half targets) separated by a 15-bp spacer within T-cell receptor alpha constant chain region (TRAC) gene were designed and produced. Each half target is recognized by repeats of the half TALE-nucleases listed in Table 12.

TABLE 12
TAL-nucleases targeting TCRalpha gene

| | Target | Repeat | Half TALE- |
|---|---|---|---|
| Target | sequence | sequence | nuclease |
| TRAC_ | TTGTCCCACA | Repeat | TRAC_T01- |
| T01 | GATATCCaga | TRAC_ | L TALEN |
| | accctgacc | T01-L | (SEQ ID |
| | ctgCCGTGTA | NG-NN-NG-HD- | NO: 40) |
| | CCAGCTGAGA | HD-HD-NI-HD- | |
| | (SEQ ID | NI-NN-NI-NG- | |
| | NO: 46) | NI-NG-HD | |
| | | Repeat | TRAC_T01- |
| | | TRAC_ | R TALEN |
| | | T01-R | (SEQ ID |
| | | HD-NG-HD-NI- | NO: 41) |
| | | NN-HD-NG-NN- | |
| | | NN-NG-NI-HD- | |
| | | NI-HD-NN | |

Each TALE-nuclease construct was subcloned using restriction enzyme digestion in a mammalian expression vector under the control of the T7 promoter. mRNA encoding TALE-nuclease cleaving TRAC genomic sequence were synthesized from plasmid carrying the coding sequence downstream from the T7 promoter.

Cryopreserved PBMC are thawed at 37° C., washed and re-suspended in Optimizer medium supplemented with AB human serum (5%) for overnight incubation at 37° C. in 5% CO2 incubator. Cells are then activated with antiCD3/CD28 coated beads in OpTmizer medium supplemented with AB human serum (5%) and recombinant human interleukin-2 (rhIL-2, 350 IU/mL) in a CO2 incubator. Three days after activation the amplified T-cells are transduced at MOI 5 with lentiviral particles vectorizing the CAR targeting CD123 (SEQ ID NO: 19). 24 hours post transduction cells are cultured in OpTmizer medium supplemented with AB human serum (5%), CTS™ Immune Cell SR (5%) rhIL-7 and rhIL-15 (culture medium). Cells are electroporated, 48 hours post transduction, with each of the 2 mRNAs encoding both half TRAC_T01 TALE-nucleases (SEQ ID NO: 40 and SEQ ID NO: 41) using AgilePulse™ Max complete system. Cells are then expanded adjusting cell concentration by adding culture medium, from time to time. On the final day of culture TCRαβ negative cells are isolated using TCRαβ biotin and anti-biotin magnetic bead system (CliniMACS TCRα/β kit) with automated and closed magnetic support cell separation system (CliniMACS Plus Instrument and CliniMACS depletion Tubing set). After depletion, cells are resuspended in culture medium. The next day cells are counted and centrifuged and resuspended in freezing medium (NaCl 0.45%, 20% human serum albumin solution, 22.5% dPBS and 7.5% DMSO).

### Example 2: Production of TCRalpha and CD52 Inactivated Cells Expressing a CD123-CAR (CD52-KO UCART123)

Heterodimeric TALE-nuclease targeting two 17-bp long sequences (called half targets) separated by a 15-bp spacer within CD52 gene were designed and produced. Each half target is recognized by repeats of the half TALE-nucleases listed in Table 13.

TABLE 13
TAL-nucleases targeting CD52 gene

| | Target | Repeat | Half TALE- |
|---|---|---|---|
| Target | sequence | sequence | nuclease |
| CD52_ | TTCCTCCTAC | Repeat | CD52_T01- |
| T01 | TCACCATcag | CD52_ | L TALEN |
| | cctcctggtt | T01-L | (SEQ ID |
| | atGGTACAGG | NG-HD-HD-NG- | NO: 42) |
| | TAAGAGCAA | HD-HD-NG-NI- | |
| | (SEQ ID | HD-NG-HD-NI- | |
| | NO: 47) | HD-HD-NI | |
| | | Repeat | CD52_T01- |
| | | CD52_ | R TALEN |
| | | T01-R | (SEQ ID |
| | | NG-NN-HD-NG- | NO: 43) |
| | | HD-NG-NG-NI- | |
| | | HD-HD-NG-NN- | |
| | | NG-NI-HD | |

Each TALE-nuclease construct was subcloned using restriction enzyme digestion in a mammalian expression vector under the control of the T7 promoter. mRNA encoding TALE-nuclease cleaving CD52 genomic sequence were synthesized from plasmid carrying the coding sequence downstream from the T7 promoter.

Cryopreserved PBMC are thawed at 37° C., washed and re-suspended in Optimizer medium supplemented with AB human serum (5%) for overnight incubation at 37° C. in 5% CO2 incubator. Cells are then activated with antiCD3/CD28 coated beads in OpTmizer medium supplemented with AB human serum (5%) (or 5% CTS™ Immune Cell SR) and recombinant human interleukin-2 (rhIL-2, 350 IU/mL) in a CO2 incubator (culture medium). Three days after activation the amplified T-cells are transduced with lentiviral particles expressing CAR targeting CD123 at MOI 5 (SEQ ID NO: 19). 24 hours post transduction cells are cultured in OpTmizer medium supplemented with AB human serum (5%), CTS™ Immune Cell SR (5%) rhIL-7 and rhIL-15 (culture medium). 48 hours post transduction, cells are electroporated with each of the 4 mRNAs encoding both half TRAC_T01 TALE-nucleases (SEQ ID NO: 40 and SEQ ID NO: 41) and both half CD52_T01 TALE-nucleases (SEQ ID NO: 42 and SEQ ID NO: 43) using AgilePulse™ Max complete system. Cells are then expanded in culture medium adjusting cell concentration, from time to time. On the final day of culture TCRαβ negative cells are isolated using TCRαβ biotin and anti-biotin magnetic bead system (CliniMACS TCRα/β kit) with automated and closed magnetic support cell separation system (CliniMACS Plus Instrument and CliniMACS depletion Tubing set). After depletion, cells are resuspended in culture medium. The next day cells are counted and centrifuged and resuspended in freezing medium (NaCl 0.45%, 20% human serum albumin solution, 22.5% dPBS and 7.5% DMSO).

### Example 3: Production of TCRalpha and B2M Inactivated Cells Expressing a CD123-CAR (B2M-KO UCART123)

Heterodimeric TALE-nuclease targeting two 17-bp long sequences (called half targets) separated by a 15-bp spacer within beta-2-microglobulin (B2M) gene were designed and produced. Each half target is recognized by repeats of the half TALE-nucleases listed in Table 14.

TABLE 14
TAL-nucleases targeting B2M gene

| | Target | Repeat | Half TALE- |
|---|---|---|---|
| Target | sequence | sequence | nuclease |
| B2M_ | TTAGCTGTGC | Repeat | B2M_T02- |
| T02 | TCGCGCTact | B2M_ | L4 TALEN |
| | ctctctttct | T02-L4 | (SEQ ID |
| | GGCCTGGAGG | NG-NI-NN-HD- | NO: 44) |
| | CTATCCA | NG-NN-NG-NN- | |
| | (SEQ ID | HD-NG-HD-NN- | |
| | NO: 48) | HD-NN-HD | |
| | | Repeat | CD52_T02- |
| | | B2M_ | R4 TALEN |
| | | T02-R4 | (SEQ ID |
| | | NN-NN-NI-NG- | NO: 45) |
| | | NI-NN-HD-HD- | |
| | | NG-HD-HD-NI- | |
| | | NN-NN-HD | |

Each TALE-nuclease construct was subcloned using restriction enzyme digestion in a mammalian expression vector under the control of the T7 promoter. mRNA encoding TALE-nuclease cleaving B2M genomic sequence were synthesized from plasmid carrying the coding sequence downstream from the T7 promoter.

Cryopreserved PBMC are thawed at 37° C., washed and re-suspended in Optimizer medium supplemented with AB human serum (5%) for overnight incubation at 37° C. in 5% CO2 incubator. Cells are then activated with antiCD3/CD28 coated beads in OpTmizer medium supplemented with AB human serum (5%) (or 5% CTS™ Immune Cell SR) and recombinant human interleukin-2 (rhIL-2, 350 IU/mL) in a CO2 incubator (culture medium). Three days after activation the amplified T-cells are transduced with lentiviral particles expressing CAR targeting CD123 at MOI 5 (SEQ ID NO: 19). 24 hours post transduction cells are cultured in OpTmizer medium supplemented with AB human serum (5%), CTS™ Immune Cell SR (5%) rhIL-7 and rhIL-15 (culture medium). 48 hours post transduction, cells are electroporated with each of the 4 mRNAs encoding both half TRAC_T01 TALE-nucleases (SEQ ID NO: 40 and SEQ ID NO: 41) and both half B2M_T02 TALE-nucleases (SEQ ID NO: 44 and SEQ ID NO: 45) using AgilePulse™ Max complete system. Cells are then expanded in culture medium adjusting cell concentration, from time to time. On the final day of culture TCRαβ negative cells are isolated using TCRαβ biotin and anti-biotin magnetic bead system (CliniMACS TCRα/β kit) with automated and closed magnetic support cell separation system (CliniMACS Plus Instrument and CliniMACS depletion Tubing set). After depletion, cells are resuspended in culture medium. The next day cells are counted and centrifuged and resuspended in freezing medium (NaCl 0.45%, 20% human serum albumin solution, 22.5% dPBS and 7.5% DMSO).

### Example 4: Production of CD123 UCART^{GT} Cells, by Inserting in Frame a CD123 CAR into the TRAC Locus

To disrupt the TRAC locus and place a CD123-specific CAR under its transcriptional control (TRAC-CAR) we used a TRAC TALEN targeting the first exon of TRAC locus and an adeno-associated virus (AAV) vector repair matrix encoding a self-cleaving T2A peptide followed by the CD123 CAR cDNA as previously described (Sachdeva et al., Granulocyte-macrophage colony-stimulating factor inactivation in CAR T-cells prevents monocyte-dependent release of key cytokine release syndrome mediator J Biol Chem. 2019 Apr. 5;294(14):5430-5437. doi: 10.1074/jbc.AC119.007558 Epub 2019 Feb. 25.; MacLeod et al., Integration of a CD19 CAR into the TCR Alpha Chain Locus Streamlines Production of Allogeneic Gene-Edited CAR T Cells, Molecular Therapy (2017), http://dx.doi.org/10.1016/j.ymthe.2017.02.00, Eyquem J, Mansilla-Soto J, Giavridis T, van der Stegen S J, Hamieh M, Cunanan K M, Odak A, Gönen M, Sadelain M, Targeting a CAR to the TRAC locus with CRISPR/Cas9 enhances tumour rejection. 2017 Mar. 2;543(7643):113-117. doi: 10.1038/nature21405. Epub 2017 Feb. 22).

PBMCs were thawed and activated using Transact human T activator CD3/CD28 beads for three days. Amplified T-cells are then transfected by electrotransfer of 1 μg per million cells of mRNA encoding TRAC TALEN (SEQ ID NO: 40 and SEQ ID NO: 41) using an AgilePulse™ Max complete system (Harvard Apparatus). Following electroporation, cells were resuspended in medium (as in example 1) and incubated at 37° C. in the presence of 5% CO2 in presence of a recombinant AAV6 donor vector, comprising in frame with the TRAC gene a self-cleaving peptide followed by CD123 CAR gene (SEQ ID NO: 19) surrounded by homology arms of the TRAC locus targeted. Subsequently, cells were cultured expanded and purified in the standard conditions. 4 days after transfection/transduction TRAC knock-out and CD123 CAR expression were assessed by flow cytometry.

TCR and CAR expressions were assessed by flow cytometry on viable T cells using CD4, CD8, TCRαβ mAb, CD123 recombinant protein fused to mouse Fc fragment in combination with a marker of cell viability.

### Example 5: TALEN®-Mediated Double Targeted Integration of Genes Encoding HLA Class I—NK Inhibitor and CAR at the B2M and TRAC Loci in Primary T-Cells, Respectively

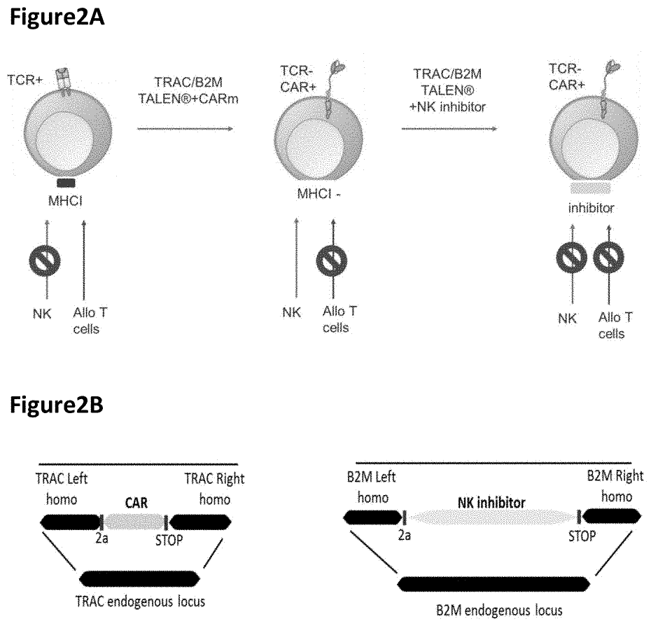

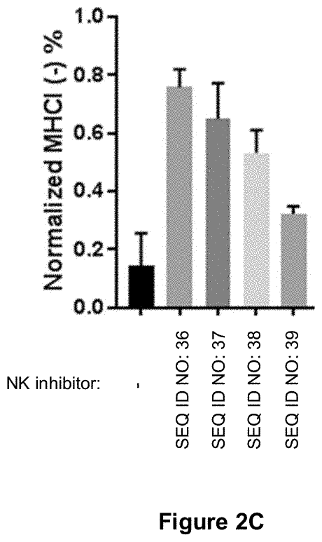

Engineered CD123 CAR T-cell with extended persistence in vivo and reduced GVHD were prepared. The method for preparing said cells consists in a simultaneous TALEN® mediated knock-out of B2M and of the TCRalpha gene (TRAC locus) in the presence of AAV6 repair vectors delivering the CD123 CAR at the TRAC locus and an NK inhibitor (i.e. HLA-E) at the B2M locus. This method prevents UCAR T123-cell to attack host tissues in a non-specific and TCR-mediated manner (TRAC KO) and to divert host T-cells mediated depletion (B2M KO) and NK-cells-mediated depletion (NK inhibitor expression) of CAR T-cells (FIG. 2A).

The method developed to integrate a NK inhibitor at the B2M locus consisted in generating a double-strand break in one of the first B2M exons using TALEN® in the presence of a DNA repair matrix vectorized by AAV6. This matrix consists of two B2M homology arms embedding the NK inhibitor coding sequence separated by a 2A cis acting elements and regulatory elements (stop codon and polyA sequences). NK inhibitors' polypeptide sequences are presented in Table 15. Because expression of B2M at the surface of CAR T-cells is likely to promote their depletion by the host immune system when transferred in an allogeneic setting, insertion of the repair matrix was designed to inactivate B2M and promote expression of the NK inhibitor.

The double targeted insertion in primary T-cells, comprises inserting the anti-CD123 CAR cDNA at the TRAC locus in the presence of TRAC TALEN®. The second matrix or exogenous gene, HLAEm is integrated as a single chain protein consisting of a fusion of B2M, HLAE peptide moiety in the B2M open reading frame using B2M TALEN®. Both matrices contained an additional 2A cis-acting element located upstream expression cassettes to enable co-expression of the single chain B2M-HLAE peptide and the CD123 CAR under endogenous B2M and TRAC promoter control, respectively (FIG. 2B).

The efficiency of double targeted insertion was measured in T-cells after transfecting the TRAC and B2M TALEN® and subsequently transducing the AAV6 repair matrices encoding either the anti-CD123 CAR surrounded by TRAC homology arms or encoding the single chain B2M-HLAE peptide surrounded by B2M homology arms. Such method led to more than 88% of TCR and B2M double knockout, to the expression of more than 68% of anti-CD123 CAR among the double knockout population and to about 68% of HLAE expression among the double knockout CAR expressing T-cells. Overall, this method enabled to generate about more than 40% of TCR/B2M negative, CAR/HLAE positive T-cells.

These engineered cells were assayed for their resistance to NK and alloresponsive T-cells attack in vitro as in PCT/EP2018/053343 and PC T/EP2018/055957, incorporated herein by reference in their entirety. The product generated was resistant to donor-specific alloreactive T cells in vitro and in vivo and to NK resistant to NK cells in vitro.

The same engineering approach was used to generate TCR/B2M negative, CAR positive T-cell bearing different NK-cell inhibitors and assess their ability to resist to NK-cell attack. The SEQ ID NO: 36 to 39 were used as NK inhibitors and tested for NK-cell resistance using an in vitro assay. Briefly, 1 million of UCART cells bearing the different NK inhibitors or not were co-cultured or not with 1 million NK cells. The impact of NK cells on the UCART cells were determined by quantification by flow cytometry of the percentage of MHC negative cells normalized to control (i.e. without NK cells condition). The results demonstrate that the tested NK inhibitors could prevent from NK-cell attack (FIG. 2C).

TABLE 15
NK inhibitors polypeptide sequences

| Product | SEQ ID # | Polypeptide sequences |
|---|---|---|
| NK | SEQ ID | MSRSVALAVLALLSLSGLEAVMAPRTLILG |
| inhibitor | NO: 36 | GGGSGGGGSGGGGSIQRTPKIQVYSRHPAE |
| 1 | | NGKSNFLNCYVSGFHPSDIEVDLLKNGERI |
| | | EKVEHSDLSFSKDWSFYLLYYTEFTPTEKD |
| | | EYACRVNHVTLSQPKIVKWDRDMGGGGSGG |
| | | GGSGGGGSGGGGSGSHSLKYFHTSVSRPGR |
| | | GEPRFISVGYVDDTQFVRFDNDAASPRMVP |
| | | RAPWMEQEGSEYWDRETRSARDTAQIFRVN |
| | | LRTLRGYYNQSEAGSHTLQWMHGCELGPDR |
| | | RFLRGYEQFAYDGKDYLTLNEDLRSWTAVD |
| | | TAAQISEQKSNDASEAEHQRAYLEDTCVEW |
| | | LHKYLEKGKETLLHLEPPKTHVTHHPISDH |
| | | EATLRCWALGFYPAEITLTWQQDGEGHTQD |
| | | TELVETRPAGDGTFQKWAAVVVPSGEEQRY |
| | | TCHVQHEGLPEPVTLRWKPASQPTIPIVGI |
| | | IAGLVLLGSVVSGAVVAAVIWRKKSSGGKG |
| | | GSYYKAEWSDSAQGSESHSL |
| NK | SEQ ID | MSRSVALAVLALLSLSGLEAVMAPRTLFLG |
| inhibitor | NO: 37 | GGGSGGGGSGGGGSIQRTPKIQVYSRHPAE |
| 2 | | NGKSNFLNCYVSGFHPSDIEVDLLKNGERI |
| | | EKVEHSDLSFSKDWSFYLLYYTEFTPTEKD |
| | | EYACRVNHVTLSQPKIVKWDRDMGGGGSGG |
| | | GGSGGGGSGGGGSGSHSLKYFHTSVSRPGR |
| | | GEPRFISVGYVDDTQFVRFDNDAASPRMVP |
| | | RAPWMEQEGSEYWDRETRSARDTAQIFRVN |
| | | LRTLRGYYNQSEAGSHTLQWMHGCELGPDR |
| | | RFLRGYEQFAYDGKDYLTLNEDLRSWTAVD |
| | | TAAQISEQKSNDASEAEHQRAYLEDTCVEW |
| | | LHKYLEKGKETLLHLEPPKTHVTHHPISDH |
| | | EATLRCWALGFYPAEITLTWQQDGEGHTQD |
| | | TELVETRPAGDGTFQKWAAVVVPSGEEQRY |
| | | TCHVQHEGLPEPVTLRWKPASQPTIPIVGI |
| | | IAGLVLLGSVVSGAVVAAVIWRKKSSGGKG |
| | | GSYYKAEWSDSAQGSESHSL |
| NK | SEQ ID | MSRSVALAVLALLSLSGLEAVMAPRTLFLG |
| inhibitor | NO: 38 | GGGSGGGGSGGGGSIQRTPKIQVYSRHPAE |
| 3 | | NGKSNFLNCYVSGFHPSDIEVDLLKNGERI |
| | | EKVEHSDLSFSKDWSFYLLYYTEFTPTEKD |
| | | EYACRVNHVTLSQPKIVKWDRDMGGGGSGG |
| | | GGSGGGGSGGGGSGSHSLKYFHTSVSRPGR |
| | | GEPRFISVGYVDDTQFVRFDNDAASPRMVP |
| | | RAPWMEQEGSEYWDRETRSARDTAQIFRVN |
| | | LRTLRGYYNQSEAGSHTLQWMHGCELGPDR |
| | | RFLRGYEQFAYDGKDYLTLNEDLRSWTAVD |
| | | TAAQISEQKSNDASEAEHQRAYLEDTCVEW |
| | | LHKYLEKGKETLLHLEPPKTHVTHHPISDH |
| | | EATLRCWALGFYPAEITLTWQQDGEGHTQD |
| | | TELVETRPAGDGTFQKWAAVVVPSGEEQRY |
| | | TCHVQHEGLPEPVTLRWKPASQPTIPIVGI |
| | | IAGLVLLGSVVSGAVVAAVIWRKKSSGGKG |
| | | GSYYKAEWSDSAQGSESHSLGSGVKQTLNF |
| | | DLLKLAGDVESNPGPMVVMAPRTLFLLLSG |
| | | ALTLTETWAGSHSMRYFSAAVSRPGRGEPR |
| | | FIAMGYVDDTQFVRFDSDSACPRMEPRAPW |
| | | VEQEGPEYWEEETRNTKAHAQTDRMNLQTL |
| | | RGYYNQSEADPPKTHVTHHPVFDYEATLRC |
| | | WALGFYPAEIILTWQRDGEDQTQDVELVET |
| | | RPAGDGTFQKWAAVVVPSGEEQRYTCHVQH |
| | | EGLPEPLMLRWKQG |
| NK | SEQ ID | MERRRGTVPLGWVFFVLCLSASSSCAVDLG |
| inhibiton | NO: 39 | SKSSNSTCRLNVTELASIHPGETWTLHGMC |
| 4 | | ISICYYENVTEDEIIGVAFTWQHNESVVDL |
| | | WLYQNDTVIRNFSDITTNILQDGLKMRTVP |
| | | VTKLYTSRMVTNLTVGRYDCLRCENGTTKI |
| | | IERLYVRLGSLYPRPPGSGLAKHPSVSADE |
| | | ELSATLARDIVLVSAITLFFFLLALRIPQR |
| | | LCQRLRIRLPHRYQRLRTEDEGRGSLLTCG |
| | | DVEENPGPMRIEWVWWLFGYFVSSVGSERS |
| | | LSYRYHLESNSSTNVVCNGNISVFVNGTLG |
| | | VRYNITVGISSSLLIGHLTIQVLESWFTPW |
| | | VQNKSYNKQPLGDTETLYNIDSENIHRVSQ |
| | | YFHTRWIKSLQENHTCDLTNSTPTYTYQVN |
| | | VNNTNYLTLTSSGWQDRLNYTVINSTHFNL |
| | | TESNITSIQKYLNTTCIERLRNYTLESVYT |
| | | TTVPQNITTSQHATTTMHTIPPNTITIQNT |
| | | TQSHTVQTPSFNDTHNVTKHTLNISYVLSQ |
| | | KTNNTTSPWIYAIPMGATATIGAGLYIGKH |
| | | FTPVKFVYEVWRGQ |

### Example 6: Production of B2M KO CLL1 UCART^{GT} Cells

Engineered CLL1 CAR T-cell with extended persistence in vivo and reduced GVHD were prepared. The method for preparing said cells consists in a simultaneous TALEN® mediated knock-out of B2M and of the TCRalpha gene (TRAC locus) in the presence of AAV6 repair vectors delivering the CLL1 CAR at the TRAC locus. This method prevents CLL1 UCART^{GT} cells to attack host tissues in a non-specific and TCR-mediated manner (graft versus host attack) and to divert host T-cells-mediated depletion of CAR T-cells.

PBMCs were thawed and activated using Transact human T activator CD3/CD28 beads for three days. Amplified T-cells are then transfected by electroporation of 1 μg per million cells of mRNA encoding TRAC TALEN (SEQ ID NO: 40 and SEQ ID NO: 41) and B2M TALEN (SEQ ID NO: 44 and SEQ ID NO: 45) using an AgilePulse™ Max complete system (Harvard Apparatus). Following electroporation, cells were resuspended in medium (as in example 1) and incubated at 37° C. in the presence of 5% CO2 in presence of a recombinant AAV6 donor vector, comprising in frame with the TRAC gene a self-cleaving peptide followed by CLL1 CAR gene (SEQ ID NO: 35) surrounded by homology arms of the TRAC locus targeted. Subsequently, cells were cultured expanded and purified in the standard conditions. 4 days after transfection/transduction TRAC knock-out and CLL1 CAR expression were assessed by flow cytometry.

TCR and CAR expressions were assessed by flow cytometry on viable T cells using CD4, CD8, TCRαβ mAb, CLL1 biotinylated recombinant protein in combination with a marker of cell viability.

### Example 7: In Vitro UCART123 Activity Against AML with Adverse Genetic Risk

Cytotoxicity of UCART123, produced in example 1, was evaluated by multi-parameter flow cytometry at 24 hours after co-cultures of AML primary samples and UCART123 or control cells (TCRα/β KO cells). The characteristics of the AML patient samples tested are indicated in Table 16.

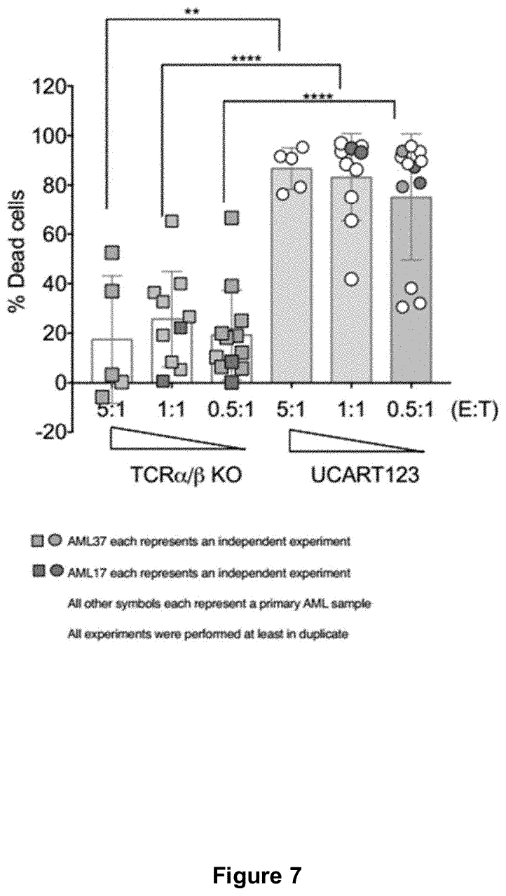

Different co-culture ratios (effector to target (E:T) ratio) were evaluated. The results show a 70% average of cell death of leukemia cells including samples from patients with AML with adverse genetic risk co-cultured with UCART123 at all E:T ratios (FIG. 7). In contrast, TCRαβ KO T-cells control cells induce significantly less cell death when co-cultured with AML cells. This result demonstrates that UCART123 is highly active on leukemia cells and in particular on leukemia cells from patients with AML with adverse genetic risk.

In addition, moderate on-target/off-tumor toxicity of UCART123 on myeloid progenitors was demonstrated using in vitro assays. The results showed that UCART123 had a mild toxicity against normal hematopoietic progenitor cells. Colony formation of erythroid cells was not affected at all, neither was colony formation for myeloid cells at E:T ratio of 0.5:1.

TABLE 16
Characteristics of primary AML samples.

| | | CD123 | |
|---|---|---|---|
| | | pos cells | |
| Sample ID | Type | (%) | AML sample information |
| *AML2 | Leukopheresis | 99.3 | Relapse; 250K WBC count; normal |
| | | | cytogenetics; FLT3-ITD; NPM1 mutant |
| *AML8 | Leukopheresis | 91.1 | NPM1 mutant |
| AML17 | Leukopheresis | 99.7 | 55 y.o. Relapse; 99.9K WBC count; normal |
| | | | cytogenetics; FLT3-ITD |
| *AML20 | Leukopheresis | 47.2 | Diagnosis; 147K WBC count; Translocation |
| | | | 11: 14; FLT3-ITD |
| AML33 | Leukopheresis | 92.8 | 61 y.o. Diagnosis; 254.6K WBC count; |
| | | | normal cytogenetics; FLT3 point mutation |
| *AML34 | Leukopheresis | 89.8 | Diagnosis; Normal cytogenetics, FLT3-ITD, |
| | | | NPM1 mutant |
| *AML37 | Leukopheresis | 86.1 | 60 y.o. Relapse; TP53 mutant, normal |
| | | | cytogenetics |
| AML40 | Bone Marrow | 86.7 | 71 y.o. Diagnosis; 91.6K WBC count; normal |
| | | | cytogenetics; FLT3-ITD |
| AML72 | Leukopheresis | 90.4 | not available |
| AML73 | Leukopheresis | 88.2 | not available |
| AML76 | Leukopheresis | 48 | Monosomy 7; DNMT3A (R882H) |
| *AML95 | Leukopheresis | 96 | 49 y.o. diagnosis. 46, XX, add(1)(p36.1), |
| | | | t(6; 11)(q27; q23)[13]/46, XX[7] |
| AML104 | Bone Marrow | 61.7 | 64 y.o. Diagnosis, 59.1 WBC count. Normal |
| | | | cytogenetics, FLT3-ITD |
| *AML105 | Bone Marrow | 61.3 | 60 y.o. diagnosis. 31.6 WBC count, |
| | | | 47~50, XY, −3, −5, del(5)(q13q33), +8, +2~4 |
| | | | mar[cp20]. TP53 (c.365_366delTG; |
| | | | p.V122Dfs*26) |

*AML with adverse genetic risk

### Example 8: In Vivo UCART123 Activity Against AML with Adverse Genetic Risk is Dependent on the Timing of Injections

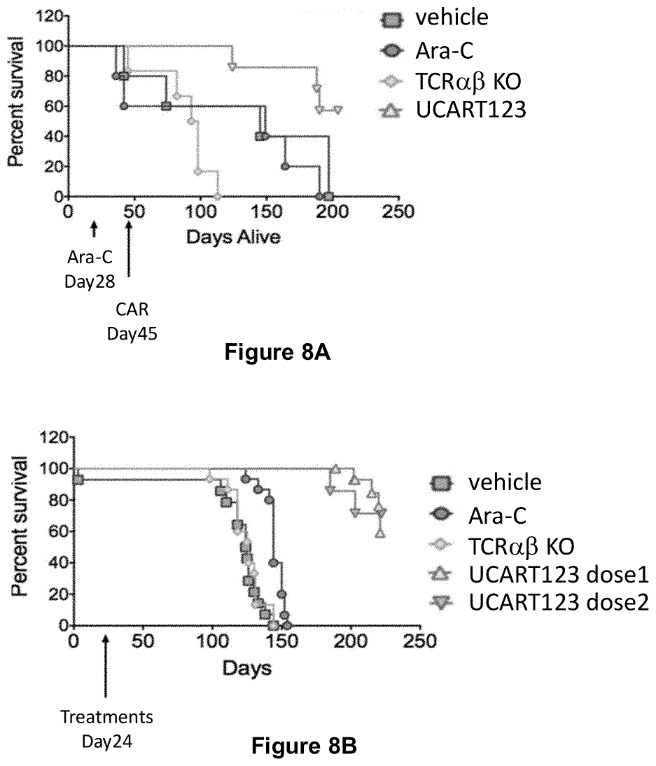

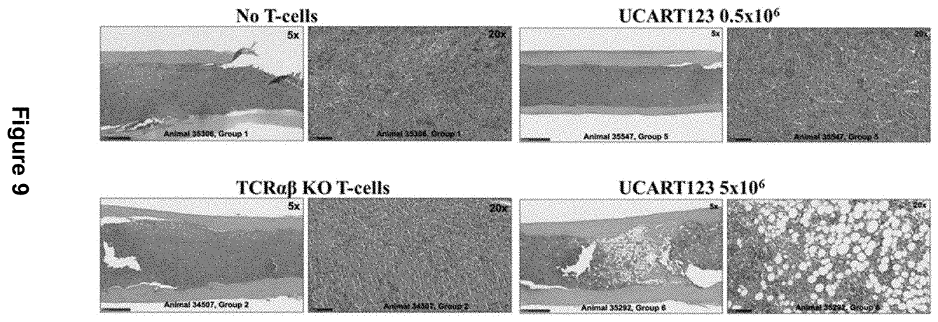

To evaluate in vivo anti-tumor activity, patient-derived xenografts (PDX) from primary AML samples were established with 2 different primary AML samples: AML37 and AML2 (both samples considered to be AML with adverse genetic risk, see Table 16). The activity of UCART123 produced in example 1 was tested in several experiments. TCRαβ KO T-cells were used as a negative control. As a chemotherapy control, a group of mice were treated with cytarabine (Ara-C) at a dose of 60 mg/kg for 5 days. Experiments with PDX-cohorts AML37 (FIG. 8A) and AML2 demonstrated (FIG. 8B) that UCART123 significantly improves overall survival. Importantly, when timing between AML and UCART123 injection was reduced from Day43 down to Day24, an improved efficacy could be observed on PDX-AML2 compared to PDX-AML37 model.

In addition, in another AML-PDX model experiments a long timing between injections of primary AML cells and UCART123 cells led to mice death with possible sign of cytokine storm. By the contrary, UCART123 was able to enhance mice survival without cytokine storm in another experiment where AML-PDX mice (engrafted with the same AML sample) were treated earlier. In this experiment, mice had less than 20% blasts in their bone marrow one day prior to UCART123-treatment.

Activity of UCART123 was also demonstrated using a primary BPDCN-PDX model in NSG mice (BPDCN sample from a 69 years old male patient with refractory/relapsed BPDCN cells). Upon engraftment, either 14 or 21 days after BPDCN injection, mice received a single injection of vehicle, 10×10⁶ TCRαβ KO T-cells (TCRαβ KO), 3×10⁶ or 10×10⁶ UCART123. Surprisingly, when BPDCN were injected 21 days prior UCART123 injection, all UCART123 treated mice died few days after treatment: 5-7 days after treatment with 10×10⁶ UCART123 injection (26-28 days after primary BPDCN sample injection) or 7-10 days after treatment with 3×10⁶ UCART123 (28-31 days after primary BPDCN sample injection). The TCRαβ KO treated mice died on Day 29-34 and the mice in Vehicle group died on Day 31-32. Mice in all groups upon sacrifice or death had very high tumor burden (engraftment of BPDCN tumor cells in peripheral blood (PB) was higher than 95%). Moreover, a high level of IFNγ was detected in peripheral blood samples from mice treated with UCART123 two days following T-cell injection. Thus, cytokine storm and/or extremely high tumor burden may have contributed to the demise of UCART123 treated mice. In sharp contrast, when BPDCN cells were injected 14 days prior UCART123 injection, all UCART123 treated mice presented an increased and dose dependent survival rate compared to vehicle or TCRαβ KO T-cells groups.

Altogether these results demonstrate the importance of managing and monitoring the tumor burden prior to UCART123 or CD52-KO UCART123 injection. The inventors have established that less than 20% of blasts detected in the bone marrow is an important criterion for a safe and highly efficient UCART123 or CD52-KO UCART123 injection especially for patient presenting AML with adverse genetic risk.

### Example 9: In Vivo Safety UCART123

### Evaluation of the Potential Toxicity of UCART123 Against Normal Hematopoietic Stem and Progenitor Cells In Vivo

NSG mice were humanized with cord blood CD34⁺ cells and 12 weeks after, were injected once or twice either with UCART123 at two different cell doses (0.5×10⁶ and 5×10⁶ cells/mouse), or TCRαβ KO T-cells from the same donor. 28 days post T-cell injection, mice were sacrificed. Histological analysis of the bone marrow samples indicates no major differences between control mice and the low dose of UCART123, while a mild (10-15%) hypocellularity in 1 out of 4 mice was observed at the high dose of UCART123 (FIG. 9). UCART cells were detected in these mice.

In Vivo Competitive BM/AML with Adverse Genetic Risk

A model where leukemic cells compete with normal hematopoietic cells as is the case in patients with leukemia, was established for testing UCART123 product for efficacy and safety. The UCART123 produced in example 1 where tested.

The xenograft model established contains both normal and leukemic cells: mixed human bone marrow T-cell depleted cells and an AML primary sample (AML2 sample from Table 16, an AML with adverse genetic risk) in sub-lethally irradiated NSG mice. Once human chimerism was confirmed, mice were injected with PBS, 1×10⁶ UCART123 cells or 1×10⁶ TCRαβ KO T-cells (Day0).

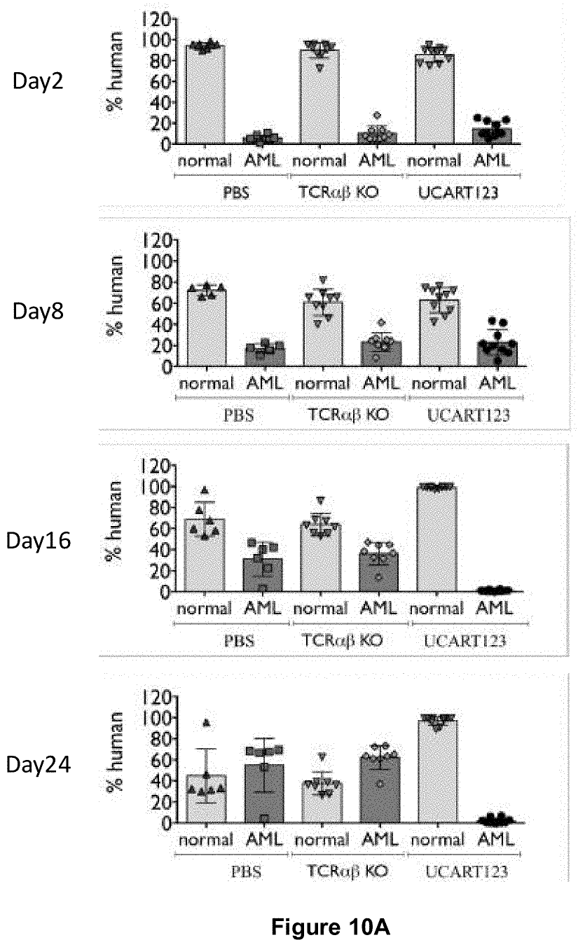

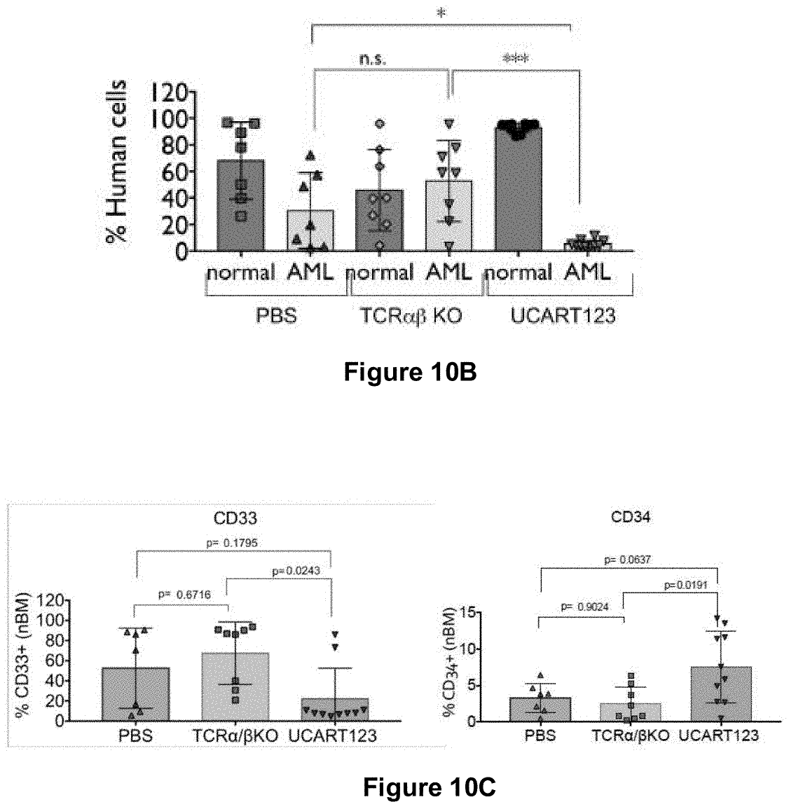

In non-treated or in control treated (with TCRαβ KO T-cells) animals, AML cells outcompete with normal cells. Indeed, AML cells could be detected in the blood with an increasing quantity from Day2 to Day24. In sharp contrast, the UCART123 treated mice showed that AML cells could be detected only at Day2 or Day8 but were almost undetectable at Day16 or Day24 whereas only normal cells could be detected in the blood (FIG. 10A).

After 5 weeks of treatment, mice were sacrificed and the Bone Marrow was evaluated by flow cytometry. Leukemic cells were selectively eliminated by UCART123 and most of the normal BM human cells were spared (FIG. 10B), while in nontreated or control treated mice, AML cells could be detected from 30% up to 50% of all human cells detected. In addition, in UCART123 treated mice, a two-fold decrease in CD33+ cells compared to control groups was detected, whereas a two-fold increase in CD34+ cells was measured (FIG. 10C).

In conclusion, in this in vivo competitive BM/AML model treated with UCART123 cells, AML cells were eliminated in the UCART123-treated mice five weeks after treatment, while progenitor cells (CD34+) were spared and only a close to 2-fold decrease of the myeloid lineage (CD33+ cells) was observed in the Bone Marrow compared to untreated or control treated mice.

Altogether these results are important for the safety of the UCART123 products but also clearly demonstrate that these products can be used in combination with a Hematopoietic Stem Cell Transplantation procedure as describe in the present invention.

### Example 11: Clinical Trial Protocol

UCART123 is a readily available, allogeneic, non-alloreactive T-cell preparation designed to become active, proliferate, secrete cytokines, and kill CD123+ blast cells following administration to lymphodepleted patients with AML.

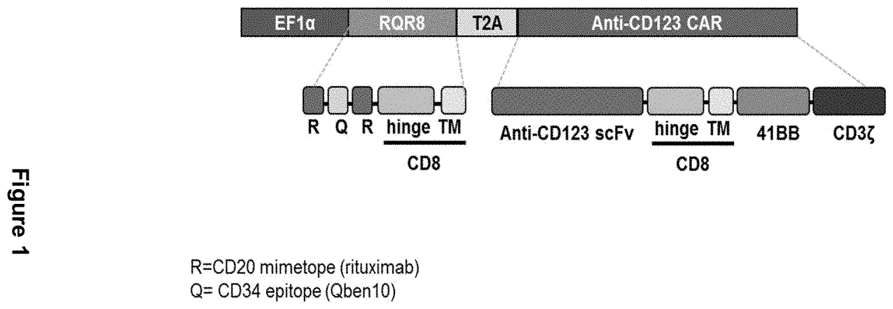

The CAR construct (in rLV or AAV6) selected for use in the present study is the following: Chimeric Antigen Receptor T-cells targeting CD123 (CD123 CAR) combining a scFv derived from an anti-CD123 antibody, from Klon43 hybridoma, the CD8 hinge and CD8 transmembrane domain, and a cytoplasmic tail composed of 4-1BB co-stimulatory and CD3 zeta signaling domains; it also comprises a 2A peptide and RQR8 motif (FIG. 1). RQR8 is a 136 amino acid artificial cell surface protein combining target epitopes from both human CD34 (to detect RQR8 using the QBend10 antibody) and human CD20 antigens to detect RQR8 using the Rituximab antibody. The expression of RQR8 on UCART123 cells permits targeted destruction of RQR8+ UCART123 cells through administration of rituximab.

Another CAR construct (in rLV or AAV6) selected for use as Chimeric Antigen Receptor T-cells targeting CD123 (CD123 CAR) combines a scFv derived from an anti-CD123 antibody, from Klon43 hybridoma humanized, a CD8 hinge and CD8 transmembrane domain, optionally at least one epitope recognized by a therapeutic antibody, rituximab and/or QBEN10, accessible extracellularly and a cytoplasmic tail composed of 4-1BB co-stimulatory and CD3 zeta signaling domains.

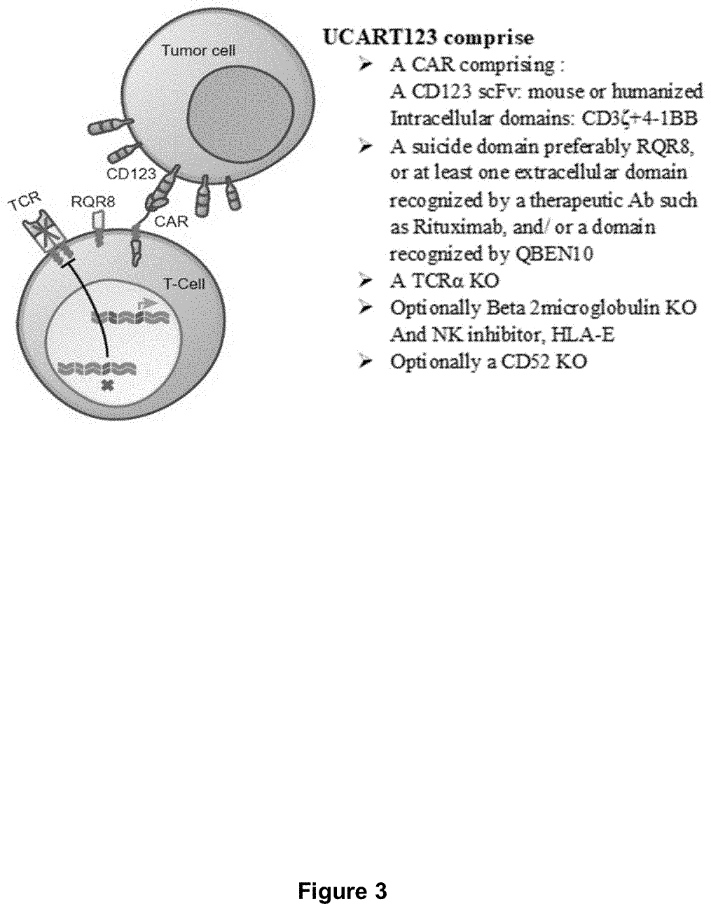

The UCART123 cells are additionally engineered to comprise at least a TALEN-inactivated TCR alpha gene with or without insertion of an exogenous encoding the CD123 CAR, optionally a TALEN-inactivated CD52 gene, and/or a TALEN-inactivated beta2 microglobulin gene and optionally a construct encoding the NK cell inhibitor, such as an HLA-E loaded-linked to a peptide (FIG. 3). TALEN® are artificially engineered nucleases that are capable of generating site-specific DNA double-strand breaks at a desired target site leading to modification (inactivation or inactivation and insertion of coding sequence) of the targeted gene.

The inactivation of the TRAC gene (encoding the TCRα subunit) results in the elimination of a functional TCRαβ at the T-cell surface. This is thought to circumvent the recognition of MHC disparities between donor and recipient through the donor cell's TCR and to prevent the potential development of graft-versus-host disease (GvHD).

The inactivation of the CD52 gene (encoding the CD52 surface molecule) results in the elimination of a CD52 at the T-cell surface. This is to make cells resistant to anti-CD52 mAb, such as alemtuzumab a therapeutic antibody targeting specifically CD52 and used for lymphodepletion.

Findings from Initial Clinical Studies
Four patients have been treated in UCART123 clinical trials, in AML123 (UCART123_01; NCT03190278), one patient in ABC123 (UCART123_02; NCT03203369). One patient was treated with UCART123 in a compassionate use (CHOP IND 17940).
At the dose-level 1 (6.25×105 CAR+ T-cells/kg) one grade 3 CRS UCART123 related and grade 4 Capillary leak syndrome (CLS) non UCART123 related were reported in one AML patient, grade 5 CRS (related to UCART123), grade 2 CLS (not related to UCART123) were reported in the BPDCN patient. One fatality was reported in the BPDCN patient.
Two other dose levels of UCART123 were explored at 6.25×10⁴ CAR+ T-cells/kg and 2.5×10⁵ CAR+ T-cells/kg. At the dose level 6.25×10⁴ CAR+ T-cells/kg, 2 patients were included and treated; no related event was reported. At the dose level 2.5×10⁵ CAR+ T-cells/kg, one grade 2 CRS UCART123 related was reported in an AML patient with recovery in less than 24 hours, after one tocilizumab administration. No GvHD was reported for any of these patients.
No GvHD was reported for any of these patients.
Rationale for UCART123 Administration in Patients with AML

The development of CAR T-cells represents a paradigm shift in the treatment of haematological malignancy; harnessing the immune system to kill leukaemia cells via targeting specific tumour antigens. Results to date have been primarily in the field of ALL with remissions demonstrated in up to 80-90% of patients in the relapsed-refractory setting (Maude et al., 2014a and 2014b). As efficacy has been demonstrated, knowledge has been gained in relation to complications particularly with respect to cytokine release syndrome (CRS). CRS has been demonstrated to correlate with leukaemia burden and severe cases of CRS are life-threatening. CAR T-cells targeting CD123 show promise in pre-clinical studies with early phase studies in the relapsed and refractory setting. The present invention provides UCART123 for use after initial cytoreduction with induction chemotherapy deepening remission. The advantage of this strategy is that UCART123 is administered whilst the patient is fit due to minimal pre-treatment chemotherapy and the low leukaemia burden minimizes the risks associated with CRS.

On the basis of the above, this invention proposes the administration of UCART123 as therapy for adverse genetic risk AML-Thus UCART123 for the treatment of adverse genetic risk AML is an object of the present invention. The strategy disclosed here aimed at delivering an efficacious therapy in patients for whom current treatment is inadequate whilst simultaneously minimizing toxicity due to administering UCART123 once or twice after initial cytoreduction where leukaemia burden has been decreased.

This trial also proposes the delivery of two doses of UCART123 designed to deepen remission prior to attempt at curative therapy with HSCT. A second lymphodepletion followed by UCART123 infusion is administered from Day 15-35 preferably day 28-35 following the first UCART123 infusion.

### Rationale for Dose Selection

The largest series describing the more recent clinical experiences using autologous CD19 CAR T cells, have employed wide ranges of infused CAR T-cell doses, from 2×10⁵/kg to 11×10⁶/kg (Kochenderfer et al., 2015; Lee et al., 2015; Maude et al., 2014a; Park et al., 2016; Porter et al., 2015).

There is limited experience with the clinical use of UCART products (allogeneic engineered T cell products). The first clinical trial with UCART19 started its recruitment in June 2016 (UCART19 PALL study, Eudract 2015-004293-15).

Three patients (two pediatric patients and one adult patient) have been treated with UCART19 supplied under an unlicensed medicinal products authorization (“specials”) granted by the MHRA. A flat dose of 4×10⁷ UCART19 CAR⁺ cells (equivalent to 4.5×10⁶/kg UCART19) was first administered, under a special authorization granted by the MHRA, to a pediatric patient (an 11 month-old infant) with refractory CD19⁺ ALL, without significant toxicity during the 28-day period following the infusion (Qasim et al., 2015). The second pediatric patient received a single dose (4×10⁶/kg) of UCART19 without any significant toxicity. Bone marrow after 28 days showed CR and was MRD negative (Qasim, ASGCT 2016).

No clinical data pertaining to CD123 targeted CAR-T therapy have been published so far; however, Budde et al., reported in February 2019 results from a first-in-human dose-escalation study (NCT02159495) of a CD123 CAR T therapy. Nine AML patients and three BPDCN patients were dosed at the flat dose levels interval between 5×10⁷ and 2×10⁸ CAR⁺ T-cells showing a rapid response consistent in 3 AML patients and in both BPDCN patients with an acceptable safety profile (Budde et al., 2019).

In the UCART123 clinical studies (AML and BPDCN) a couple of dose levels have been tested: 6.25×10⁵ CD123CAR+_T-cells/kg, 6.25×10⁴ CD123CAR+_T-cells/kg and 2.5×10⁵ CD123CAR+_T-cells/kg. Given the published data with autologous CAR T-cells and the doses investigated with UCART19 and UCART123, a dose of 2.5×10⁵ CD123CAR+_T-cells/kg is recommended to start the dose escalation. This dose is below the dose of 1×10⁶ per kg of body weight that was identified as the recommended dose in the series published in Lee et al., 2015.

Considering that the patients targeted in this present UCART123 protocol are at a lower risk of CRS (minimal pre-treatment chemotherapy and low leukaemia burden minimizing the risks associated with CRS) and are selected according to stringent criteria, the dose-level 1 of the dose-escalation phase for this trial is 6.25×10⁵ UCART123/kg which corresponds to a dose sufficient for UCART123 expansion.

### Rationale for a Second UCART123 Administration

This trial proposes the delivery of at least two doses of UCART123 designed to deepen remission prior to attempt at curative therapy with HSCT. Thus, for patients who did not achieve a morphological CR with negative MRD (defined as MRD <0.01% by flow cytometry or molecular methods) after the initial UCART123 administration, a second UCART123 infusion is administered, provided no Dose Limiting Toxicity (DLT) has been observed and clinical safety parameters are met.

### Benefit/Risk Assessment

The data and safety monitoring board (DSMB) review the safety data from each cohort after all patients in the cohort have completed their DLT observation period and recommend to proceed to the next ascending dose-level, to de-escalate or to add a new cohort of patients at the same dose-level to further evaluate the safety of the second dose. For each previously untested dose-level, only one patient was initially treated to check the absence of life-threatening toxicity at this dose. After a minimum period of 2 weeks (to cover for the period in which CRS is most likely to occur), subsequent patients may be treated.

### Potential Risks

The safety risks potentially associated with the administration of UCART123 are those expected from cytotoxic chemotherapy and those described for other CAR T-cells. Chemotherapy is the mainstay of treatment for AML, and expected toxicities include transfusion-dependent myelosuppression, neutropenic infections, bleeding, and multi-organ toxicities. Life-threatening complications are not uncommon with standard AML chemotherapy. Patients who are receiving a second UCART123 administration after Day 28 will be administered a new lymphodepletion regimen, according to the same modalities as for the first administration.

Potential toxicities related to UCART123 include, but are not limited to:
CRS;
TLS;
Neurologic toxicities including obtundation, seizures, aphasia/dysphasia, and mental status changes;
On-target/off-tumor toxicity (e.g., depletion of normal cells such as HSCs expressing CD123 with subsequent myelosuppression or occurrence of a capillary leak syndrome [CLS] due to the expression of CD123 on endothelial cells);
Graft-versus-host disease cannot be completely excluded despite the depletion of TCRαβ expressing T-cells in the UCART123 preparation. In two ongoing Phase I ALL studies with the similar allogeneic CAR T-cell product UCART19, only Grade I skin-restricted GvHD symptoms have been observed to date, in ⅕ pediatric ALL patients and 1/7 adult ALL patients (Qasim et al., 2017; Benjamin et al., 2017);
Generation of replication competent lentivirus (RCL) which is very unlikely; and
Malignant transformation of UCART123 due to genomic modifications associated with the use of lentiviral or adeno vectors and/or TRAC TALEN® alone or in combination with CD52 or B2M TALEN®.

### Benefits

The primary benefit to be observed from UCART123 for participating patients is a high degree of T-cell expansion that induced high and sustained anti-CD123 activity, leading to durable remission in poor-prognosis patients with AML. The delivery of two doses of UCART123 is designed to deepen remission prior to attempt at curative therapy with HSCT. Also, patients are expected to benefit from the immediate availability of UCART123 cells and the higher, more homogenous transduction success rate expected from healthy allogeneic cells, compared to autologous T-cells. The absence of cell-surface expression of the TCR complex on UCART123 eliminates the TCR-recognition of histocompatibility antigens, the primary mechanism of GVHD, and confers a “universal” character to UCART123.

### Study Objectives

The primary objectives of the study were:
To evaluate the safety and tolerability of a multiple infusion schedule of UCART123.
To determine the Maximum Tolerated Dose (MTD) of UCART123.

### Dose-Escalation Procedure

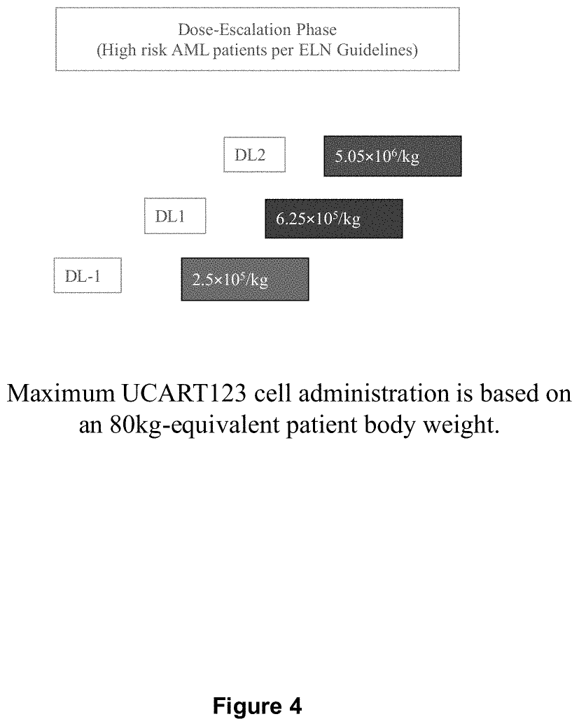

This is a dose escalation study where three UCART123 dose-levels are tested (FIG. 4). The dose allocation starts at the dose-level 1.

### Determination of the MTD

The MTD is the dose with estimated probability of toxicity the closest to the target toxicity rate, among all tested dose-levels not excluded for over toxicity.

### Safety Endpoint

Incidence, nature, and severity of adverse events and serious adverse events (SAEs) throughout the study are monitored according to Lee et al., 2014 for CRS; Cairo and Bishop 2004 for TLS, Harris et al., 2016 for GvHD).

### Efficacy Endpoints

Antileukemic activity, as measured by European Leukaemia Net (ELN) Response Criteria in AML (Döhner et al., 2017). Response is assessed following each UCART123 administration at Day 14 and Day 28, at the end of treatment visit and as clinically relevant.

### The Study Recruitment is Based on ELN Criteria

Patients newly diagnosed with CD123 positive adverse genetic risk acute myeloid leukaemia (AML), including patients with CD123 positive AML secondary to MDS, who do not achieve complete remission, and whose bone marrow blast content is <20% blasts after 1 or 2 courses of standard intensive induction chemotherapy. Adverse genetic risk is defined as per ELN guidelines (Döhner et al., 2017):
a. t(6;9)(p23;q34.1); DEK-NUP214; or
b. t(v;11q23.3); KMT2A rearranged; or
c. t(9;22)(q34.1;q11.2); BCR-ABL1; or
d. inv(3)(q21.3q26.2) or t(3;3)(q21.3;q26.2); GATA2, MECOM(EVI1); or
e. -5 or del(5q); -7; -17/abn(17p) or Complex karyotype
Three or more unrelated chromosome abnormalities in the absence of one of the World Health Organization-designated recurring translocations or inversions, i.e., t(8;21), inv(16) or t(16;16), t(9;11), t(v;11)(v;q23.3), t(6;9), inv(3) or t(3;3); AML with BCR-ABL1; or
f. Monosomal karyotype
Presence of one single monosomy (excluding loss of X or Y) in association with at least one additional monosomy or structural chromosome abnormality (excluding core-binding factor AML); or
h. Wild-type NPM1 and FLT3-ITD high or
i. Mutated RUNX1 (except if co-occur with favorable-risk AML subtypes) or
j. Mutated ASXL1 (except if co-occur with favorable-risk AML subtypes) or
k. Mutated TP53
   Availability of a suitable sibling or unrelated HLA matched donor;

### Study Design and Schedule

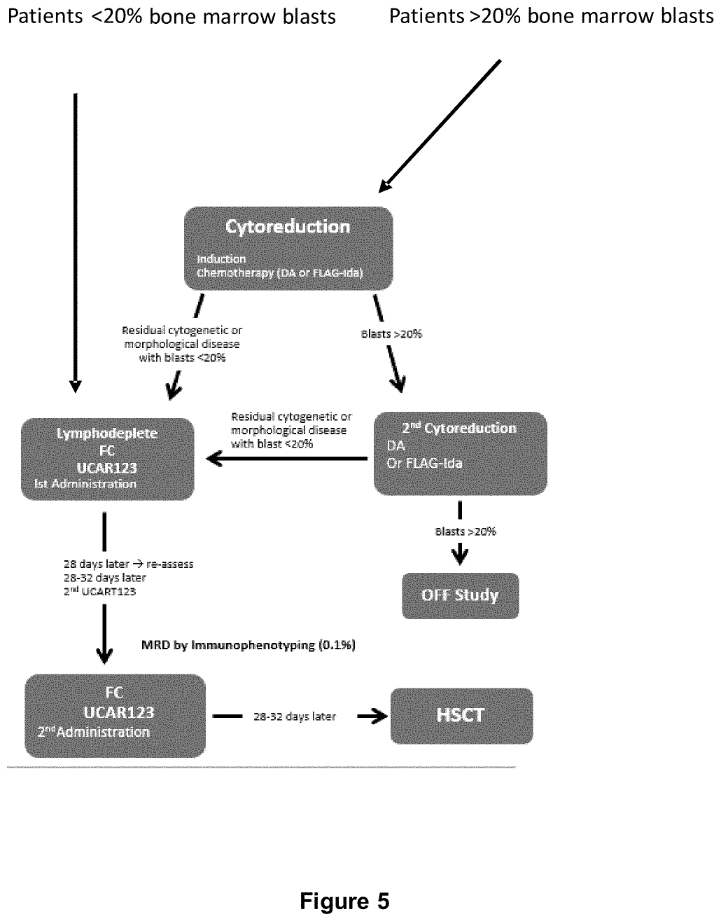

This is a Phase I, open-label, dose-finding study of UCART123 administered intravenously to patients with CD123+ adverse genetic risk AML. The study consists of a dose-escalation phase in patients newly diagnosed with CD123 positive adverse genetic risk acute myeloid leukaemia (AML) defined in the ELN adverse genetic risk group (Döhner et al., 2017); who do not achieve morphologic or cytogenetic complete remission after standard intensive induction chemotherapy (FIG. 5).

Patients with less than 20% blasts are treated with a lymphodepleting regimen. Subsequently, the dose-escalation phase is explored using two doses of UCART123 ranging from 6.25×10⁵ cells/kg to 5.05×10⁶ cells/kg. The lymphodepleting regimen can be modified (either in composition or in doses) and adapted during the study based upon safety, biological, and/or clinical activity observations.

Based on the observed safety, efficacy and cell expansion kinetics of UCART123 the lymphodepletion regimen can be adjusted with the use of an anti-CD52 therapy (through a specific protocol amendment) to increase the depth and duration of lymphodepletion and enhance UCART123 expansion.

Patients are considered for a HSCT after a single UCART123 infusion, if: (i) they experienced any DLTs during the DLT observation period, or (ii) they achieved CR with MRD <0.01% (by flow cytometry or molecular methods). All other patients are considered for a second UCART123 dose after Day 28 at the same dose-level of UCART123 as for their first administration following a second lymphodepletion. For the second UCART123 administration, the patient must have recovered from all acute toxicities of the first lymphodepletion regimen and first infusion of UCART123.

Of note, the safety and efficacy data are analyzed during the overall study duration but the determination of the MTD is based only on the results of DLT observation period.

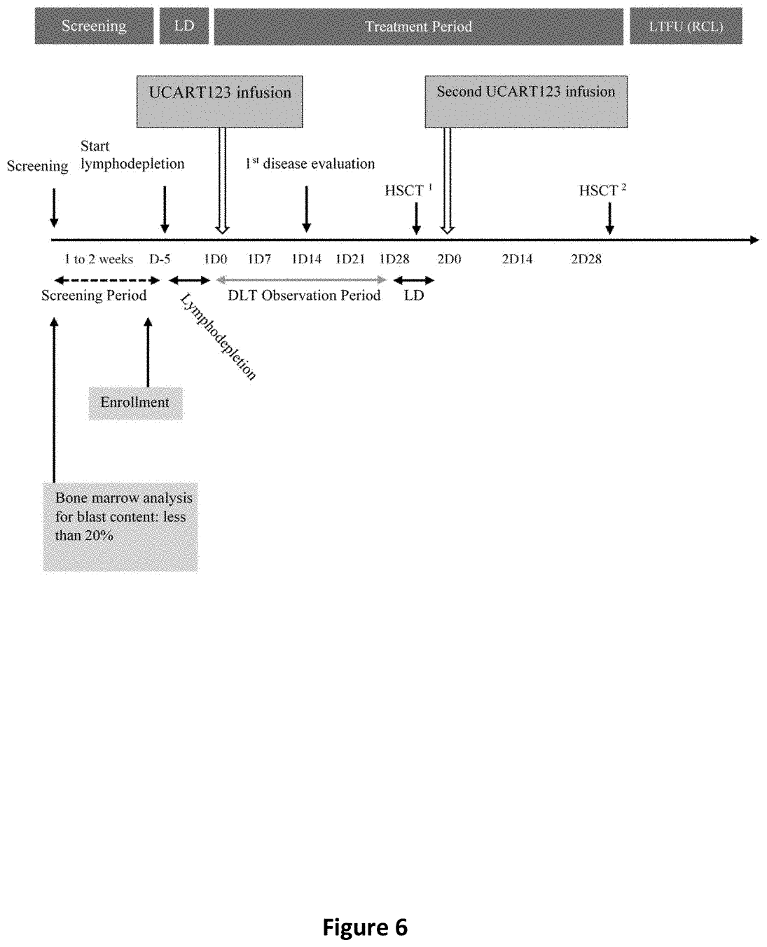

Study Schedule is presented in FIG. 6.

### REFERENCES

Benjamin R., Graham C., Yallop D., Jozwik A., Patten P., Dunlop A., Ellard R., Stewart O., et al. Preliminary results of UCART19, an allogeneic anti-CD19 CAR Tcell product, in a first-in-human trial (CALM) in adult patients with CD19+ relapsed/refractory B-cell acute lymphoblastic leukemia. American Society of Gene and Cell Therapy, 2017. Atlanta.
Budde E., oral presentation, 1st European CART Cell Meeting, 2019. Paris.
Brentjens, R. J., Davila, M. L., Riviere, I., Park, J., Wang, X., Cowell, L. G., Bartido, S., Stefanski, J., Taylor, C., Olszewska, M., et al. (2013). CD19-targeted T cells rapidly induce molecular remissions in adults with chemotherapy-refractory acute lymphoblastic leukemia. Sci. Transl. Med. 5, 177ra38.
Cairo, M. S., and Bishop, M. (2004). Tumour lysis syndrome: new therapeutic strategies and classification. Br. J. Haematol. 127, 3-11.
Carpenito, C., Milone, M. C., Hassan, R., Simonet, J. C., Lakhal, M., Suhoski, M. M., Varela-Rohena, A., Haines, K. M., Heitjan, D. F., Albelda, S. M., et al. (2009). Control of large, established tumor xenografts with genetically retargeted human T cells containing CD28 and CD137 domains. Proc. Natl. Acad. Sci. U.S.A 106, 3360-3365.
Davila, M. L., Bouhassira, D. C. G., Park, J. H., Curran, K. J., Smith, E. L., Pegram, H. J., and Brentjens, R. (2014a). Chimeric antigen receptors for the adoptive T cell therapy of hematologic malignancies. Int. J. Hematol. 99, 361-371.
Della Porta, M. G., Alessandrino, E. P., Bacigalupo, A., van Lint, M. T., Malcovati, L., Pascutto, C., Falda, M., Bernardi, M., et al. Predictive factors for the outcome of allogeneic transplantation in patients with MDS stratified according to the revised IPSS-R. Blood 2014 123:2333-2342.
Döhner, H., Estey, E., Grimwade, D., Amadori, S., Appelbaum, F. R., Büchner, T., Dombret, H., Ebert, B. L., Fenaux, P., Larson, R. A., et al. (2017). Diagnosis and management of AML in adults: 2017 ELN recommendations from an international expert panel. Blood. 129, 424-447.
Döhner, H., Estey, E. H., Amadori, S., Appelbaum, F. R., Büchner, T., Burnett, A. K., Dombret, H., Fenaux, P., Grimwade, D., Larson, R. A., et al. (2010). Diagnosis and management of acute myeloid leukemia in adults: recommendations from an international expert panel, on behalf of the European LeukemiaNet. Blood 115, 453-474.
European Parliament E, Council of the European Union (EC), Directorate-General for Enterprise and I
Estey, E. H. (2014). Acute myeloid leukemia: 2014 update on risk-stratification and management. Am. J. Hematol. 89, 1063-1081.
Estey, E., and Döhner, H. (2006). Acute myeloid leukaemia. Lancet Lond. Engl. 368, 1894-1907.
Forman, S. J., and Rowe, J. M. (2013). The myth of the second remission of acute leukemia in the adult. Blood 121, 1077-1082.
Frankel, A., Liu, J.-S., Rizzieri, D., and Hogge, D. (2008). Phase I clinical study of diphtheria toxin-interleukin 3 fusion protein in patients with acute myeloid leukemia and myelodysplasia. Leuk. Lymphoma 49, 543-553
Goel, S., Hall, J., Pradhan, K., Hirsch, C., Przychodzen, B., Shastri, A., Mantzaris, I., Janakiram, M., Battini, R., Kornblum, N., et al. (2016). High prevalence and allele burden-independent prognostic importance of p53 mutations in an inner-city MDS/AML cohort. Leukemia 30, 1793-1795.
Harris, A. C., Young, R., Devine, S., Hogan, W. J., Ayuk, F., Bunworasate, U., Chanswangphuwana, C., Efebera, Y. A., Holler, E., Litzow, M., et al. (2016). International, Multicenter Standardization of Acute Graft-versus-Host Disease Clinical Data Collection: A Report from the Mount Sinai Acute GVHD International Consortium. Biol. Blood Marrow Transplant. J. Am. Soc. Blood Marrow Transplant. 22, 4-10.
He, S. Z., Busfield, S., Ritchie, D. S., Hertzberg, M. S., Durrant, S., Lewis, I.D., Marlton, P., McLachlan, A. J., Kerridge, I., Bradstock, K. F., et al. (2015). A Phase 1 study of the safety, pharmacokinetics and anti-leukemic activity of the anti-CD123 monoclonal antibody CSL360 in relapsed, refractory or high-risk acute myeloid leukemia. Leuk. Lymphoma 56, 1406-1415.
Kochenderfer, J. N., Dudley, M. E., Kassim, S. H., Somerville, R. P. T., Carpenter, R. O., Stetler-Stevenson, M., Yang, J. C., Phan, G. Q., Hughes, M. S., Sherry, R. M., et al. (2015). Chemotherapy-refractory diffuse large B-cell lymphoma and indolent B-cell malignancies can be effectively treated with autologous T cells expressing an anti-CD19 chimeric antigen receptor. J. Clin. Oncol. Off. J. Am. Soc. Clin. Oncol. 33, 540-549.
Imai, C., Mihara, K., Andreansky, M., Nicholson, I. C., Pui, C.-H., Geiger, T. L., and Campana, D. (2004). Chimeric receptors with 4-1BB signaling capacity provoke potent cytotoxicity against acute lymphoblastic leukemia. Leukemia 18, 676-684.
Kochenderfer, J. N., Dudley, M. E., Kassim, S. H., Somerville, R. P. T., Carpenter, R. O., Stetler-Stevenson, M., Yang, J. C., Phan, G. Q., Hughes, M. S., Sherry, R. M., et al. (2015). Chemotherapy-refractory diffuse large B-cell lymphoma and indolent B-cell malignancies can be effectively treated with autologous T cells expressing an anti-CD19 chimeric antigen receptor. J. Clin. Oncol. Off. J. Am. Soc. Clin. Oncol. 33, 540-549.
Lee, D. W., Gardner, R., Porter, D. L., Louis, C. U., Ahmed, N., Jensen, M., Grupp, S. A., and Mackall, C. L. (2014). Current concepts in the diagnosis and management of cytokine release syndrome. Blood 124, 188-195.
Lee, D. W., Kochenderfer, J. N., Stetler-Stevenson, M., Cui, Y. K., Delbrook, C., Feldman, S. A., Fry, T. J., Orentas, R., Sabatino, M., Shah, N. N., et al. (2015). T cells expressing CD19 chimeric antigen receptors for acute lymphoblastic leukaemia in children and young adults: a phase 1 dose-escalation trial. Lancet Lond. Engl. 385, 517-528.
Lindsley, R. C., Saber, W., Mar, B. G., Redd, R., Wang, T., Haagenson, M. D., Grauman, P. V., Hu, Z.-H., Spellman, S. R., Lee, S. J., et al. (2017). Prognostic Mutations in Myelodysplastic Syndrome after Stem-Cell Transplantation. N. Engl. J. Med. 376, 536-547.
Majeti, R. (2011). Monoclonal antibody therapy directed against human acute myeloid leukemia stem cells. Oncogene 30, 1009-1019.
Maude, S. L., Frey, N., Shaw, P. A., Aplenc, R., Barrett, D. M., Bunin, N.J., Chew, A., Gonzalez, V. E., Zheng, Z., Lacey, S. F., et al. (2014a). Chimeric antigen receptor T cells for sustained remissions in leukemia. N. Engl. J. Med. 371, 1507-1517.
Maude, S. L., Barrett, D., Teachey, D. T., and Grupp, S. A. (2014b). Managing cytokine release syndrome associated with novel T cell-engaging therapies. Cancer J. Sudbury Mass 20, 119-122.
Middeke, J. M., Herold, S., Rücker-Braun, E., Berdel, W. E., Stelljes, M., Kaufmann, M., Schafer-Eckart, K., Baldus, C. D., Stuhlmann, R., Ho, A. D., et al. (2016). TP53 mutation in patients with high-risk acute myeloid leukaemia treated with allogeneic haematopoietic stem cell transplantation. Br. J. Haematol. 172, 914-922.
Park, J. H., Geyer, M. B., and Brentjens, R. J. (2016). CD19-targeted CAR T-cell therapeutics for hematologic malignancies: interpreting clinical outcomes to date. Blood 127, 3312-3320.
Porter, D. L., Hwang, W.-T., Frey, N. V., Lacey, S. F., Shaw, P. A., Loren, A. W., Bagg, A., Marcucci, K. T., Shen, A., Gonzalez, V., et al. (2015). Chimeric antigen receptor T cells persist and induce sustained remissions in relapsed refractory chronic lymphocytic leukemia. Sci. Transl. Med. 7, 303ra139.
Qasim W., P. J. Amrolia, S. Samarasinghe, S. Ghorashian, H. Zhan, S. Stafford, K. Butler, G. Ahsan, K. Gilmour, S. Adams, D. Pinner, R. Chiesa, S. Chatters, S. Swift, N. Goulden, K. Peggs, A. J. Thrasher, P. Veys, and M. Pule. 2046: First Clinical Application of Talen Engineered Universal CAR19 T Cells in B-ALL. Cellectis. Anonymous. Orlando, Fla., United States:57th Annual Meeting of the American Society of Hematology (ASH). 2015.
Qasim W., Universal CAR T cell Bridge to transplantation, American Society of Gene and Cell Therapy, 2016. Washington.
Qasim W., Ciocarlie O., Adams S., Inglott S., Murphy C., Rivat C., Wright G., et al. Preliminary Results of UCART19, an Allogeneic Anti-CD19 CAR T-Cell Product in a First-in-Human Trial (PALL) in Pediatric Patients with CD19+ Relapsed/Refractory B-Cell Acute Lymphoblastic Leukemia. American Society of Gene and Cell Therapy, 2017. Atlanta.
Roboz, G. J. (2012). Current treatment of acute myeloid leukemia. Curr. Opin. Oncol. 24, 711-719.
Roboz, G. J., Rosenblat, T., Arellano, M., Gobbi, M., Altman, J. K., Montesinos, P., O'Connell, C., Solomon, S. R., Pigneux, A., Vey, N., et al. (2014). International randomized phase III study of elacytarabine versus investigator choice in patients with relapsed/refractory acute myeloid leukemia. J. Clin. Oncol. Off. J. Am. Soc. Clin. Oncol. 32, 1919-1926.
Röllig, C., Bornhäuser, M., Thiede, C., Taube, F., Kramer, M., Mohr, B., Aulitzky, W., Bodenstein, H., Tischler, H.-J., Stuhlmann, R., et al. (2011). Long-term prognosis of acute myeloid leukemia according to the new genetic risk classification of the European LeukemiaNet recommendations: evaluation of the proposed reporting system. J. Clin. Oncol. Off. J. Am. Soc. Clin. Oncol. 29, 2758-2765.
Rücker, F. G., Schlenk, R. F., Bullinger, L., Kayser, S., Teleanu, V., Kett, H., Habdank, M., Kugler, C.-M., Holzmann, K., Gaidzik, V. I., et al. (2012). TP53 alterations in acute myeloid leukemia with complex karyotype correlate with specific copy number alterations, monosomal karyotype, and dismal outcome. Blood 119, 2114-2121.
Yanada, M., Yamamoto, Y., Iba, S., Okamoto, A., Inaguma, Y., Tokuda, M., Morishima, S., Kanie, T., Mizuta, S., Akatsuka, Y., et al. (2016). TP53 mutations in older adults with acute myeloid leukemia. Int. J. Hematol. 103, 429-435.
Yoshizato, T., Nannya, Y., Atsuta, Y., Shiozawa, Y., Iijima-Yamashita, Y., Yoshida, K., Shiraishi, Y., Suzuki, H., Nagata, Y., Sato, Y., et al. (2017). Genetic abnormalities in myelodysplasia and secondary acute myeloid leukemia: impact on outcome of stem cell transplantation. Blood 129, 2347-2358.

1. A method for treating a patient having an adverse genetic risk of AML by cell immunotherapy, said method comprising:
i) subjecting the patient to induction chemotherapy treatment to reduce blasts in bone marrow of the patient to lower than 20%; wherein minimal residual disease (MRD) is not achieved;
ii) subjecting the patient to lymphodepleting treatment to reduce the patient's own immune cells;
iii) subjecting the patient to immunotherapy treatment comprising administering a dose of engineered immune cells expressing a chimeric antigen receptor (CAR) or a recombinant TCR specific for a tumoral antigen marker at the surface membrane of said remaining blasts to achieve MRD; and
iv) optionally, administering a second dose of engineered immune cells expressing a chimeric antigen receptor (CAR) until reaching actual MRD;
v) optionally, treating the patient with a pre-conditioning regimen prior to bone marrow transplant; and/or
vi) optionally, proceeding to a bone marrow transplant.

2. The method according to claim 1, wherein the patient has at least one genetic marker selected from:
t(6;9)(p23;q34.1); DEK-NUP214;
t(v;11q23.3); KMT2A rearranged;
t(9;22)(q34.1;q11.2); BCR-ABL1;
inv(3)(q21.3q26.2) or t(3;3)(q21.3;q26.2); GATA2, MECOM(EVI1);
-5 or del(5q); -7; -17/abn(17p);
Complex karyotype comprising three or more unrelated chromosome abnormalities in the absence of the following recurring translocations or inversions: t(8;21), inv(16) or t(16;16), t(9;11), t(v;11)(v;q23.3), t(6;9), inv(3), or t(3;3), or AML with BCR-ABL1;
Monosomal karyotype presenting one single monosomy, excluding loss of X or Y, in association with at least one additional monosomy or structural chromosome abnormality, excluding core-binding factor AML;
Wild-type NPM1 and FLT3-ITD high;
Mutated RUNX1 that does not co-occur with a favorable-risk AML subtype;
Mutated ASXL1 that does not co-occur with a favorable-risk AML subtype; and
Mutated TP53.

3. The method according to claim 1, wherein said engineered immune cells express a CAR specific for a tumoral antigen selected from CD25, CD30, CD37, CD38, CD33, CD47, CD98, CD123, FLT3, CLL-1, CD56, CD117, CD133, CD157, c-kit, CD34, MUC1, CXCR4, VEGF, NKG2D_F, folate receptor beta (FR beta), hepatocyte growth factor (HGF), HLA-A2, and Lewis Y.

4. The method according to claim 1, wherein said engineered immune cells express a CAR specific for CD123 and/or CLL1 tumoral antigen(s).

5. (canceled)

6. The method according to claim 1, wherein at least 80% of said engineered immune cells are TCRαβ−_T-cells, anti-CD123 CAR+_TCRαβ−_T-cells, and/or anti-CLL-1 CAR+_TCRαβ−_T-cells.

7. The method according to claim 1, wherein said engineered immune cells have been genetically engineered using rare-cutting endonuclease(s) or TALE-nucleases.

8. The method according to claim 1, wherein said engineered immune cells comprise at least one of the following DNA modifications: an exogenous DNA sequence encoding a CAR inserted into the genome, an exogenous DNA sequence encoding a CAR inserted into the genome at a TCR locus, an exogenous DNA sequence encoding an NK inhibitor an exogenous DNA sequence encoding an HLA-E-peptide fusion peptide inserted into the genome, an alpha TCR KO gene, a B2M KO gene, a CD52 KO, and any combination thereof.

9. The method according to claim 1, wherein said engineered immune cells comprise more than 40% and up to 99% TCRalpha, beta negative and CD52 negative cells, or more than 40% and up to 99% of TCRalpha, beta negative and beta2microglobulin negative cells or more than 40% and up to 88% TCRalpha, beta negative, CD52 negative, beta2 microglobulin negative cells.

10. The method according to claim 1, wherein said engineered immune cells comprise more than 40% and up to 99% CAR+/HLA-E+ cells.

11. The method according to claim 1, wherein said engineered immune cells comprise less than 5% TCR-positive cells.

12. The method according to claim 1, wherein said induction chemotherapy treatment is selected from:
a combination of an anthracycline and cytarabine;
anti CD33 antibody;
a protein kinase inhibitor in combination with cytarabine and/or daunorubicin;
a combination of Venetoclax with azacytidine and/or decitabine and/or cytarabine; and
a combination of Glasdegib with cytarabine.

13. The method according to claim 1, wherein the induction chemotherapy treatment comprises a 3+7 regimen comprising 3 days of an IV combination of anthracycline, daunorubicin and idarubicin and/or mitoxantrone, and 7 days of continuous infusion of cytarabine; or a FLAG Ida regimen comprising Fludarabine, Cytarabine, Idarubicin and G-CSF.

14-15. (canceled)

16. The method according to claim 1, wherein said lymphodepleting treatment comprises fludarabine and Cyclophosphamide, wherein fludarabine is administered at a dose from about 20 to about 60 mg/m²/day and Cyclophosphamide is administered at a dose of from about 1 to about 2 g/m²/day.

17. The method according to claim 1, wherein said lymphodepleting treatment comprises an anti-CD52 drug.

18. The method according to claim 1, wherein the engineered immune cells are administered at a dose of about 10⁴ to about 10⁷ cells/kg.

19. The method according to claim 1, wherein at least two doses of engineered immune cells expressing a CAR specific for a tumoral antigen are administered after the lymphodepletion treatment.

20. The method according to claim 1, wherein haematopoietic stem cells used for the bone marrow transplant of step vi) are HLA matching to the engineered immune cells of step iii).

21. A method for achieving remission of a hematological cancer in a patient comprising:
a) identifying a patient with a hematological cancer with adverse cytogenetic risk;
b) measuring blasts content over total cells in a sample of the bone marrow of said patient; wherein if blast content is less than 20% over total cells in the bone marrow step (d) is performed;
c) wherein if blast content is more than 20% over total cells in the bone marrow, at least one debulking treatment(s) is administered to reach less than 20% blasts in the bone marrow;
d) lymphodepleting said patient and administering one dose of engineered immune cells expressing a CAR specific for a tumoral antigen marker at the cell surface membrane of said remaining blasts; and
e) measuring blasts in the bone marrow;

22. The method according to claim 21, further comprising:
f) if Minimal Residual Disease (MRD) is not achieved, the method comprises administering a second lymphodepleting treatment and administering a second dose of engineered immune cells;
g) if MRD is <0.1%, the method comprises transplanting bone marrow stem cells from a compatible donor.

23. A method for monitoring a patient being treated for adverse genetic risk AML using engineered CAR positive immune cells and a plurality of stem cells, wherein said method comprises the steps of:
a) vivo analyzing, ex vivo, blasts from the patient's bone marrow sample, said patient having been pre-treated by induction chemotherapy;
b) wherein if blast count is between 1 and 20% in the sample, the method comprises preparing ex-vivo engineered CAR positive immune cells directed against an antigen marker present on said blasts;
c) optionally, two weeks after treating the patient with the engineered CAR positive immune cells, analyzing the blasts ex vivo from patient's bone marrow sample by flow cytometry to determine whether MRD is reached if not, the method further comprises providing ex-vivo engineered CAR positive immune cells of step b) in view of a second round of treatment;
d) optionally, if MRD is reached in step c), the method comprises providing stem cells from a compatible donor in view of a transplant.

24-37. (canceled)

38. A medical kit comprising at least a first and second composition for sequential use for treating AML, wherein said first composition is used for induction chemotherapy to reduce or maintain blasts in bone marrow between 1 and 20%, and wherein said second composition comprises a dose of engineered immune cells expressing a chimeric antigen receptor (CAR) specific for a tumoral antigen at the cell surface membrane.

39. The medical kit according to claim 38, wherein said second composition is a therapeutic composition comprising a dose of engineered immune cells expressing a CAR+_TCRαβ−T-specific for a tumoral antigen selected from CD25, CD30, CD37, CD38, CD33, CD47, CD98, CD123, FLT3, CLL-1, CD56, CD117, CD133, CD157, c-kit, CD34, MUC1, CXCR4, VEGF, NKG2D_F, folate receptor beta (FR beta), hepatocyte growth factor (HGF), HLA-A2, and Lewis Y.


SEQUENCE LISTING (48 sequences)

SEQ ID NO: 1 - protein, 21 aa, Homo sapiens
MALPVTALLLPLALLLHAARP

SEQ ID NO: 2 - protein, 20 aa, Homo sapiens
METDTLLLWVLLLWVPGSTG

SEQ ID NO: 3 - protein, 16 aa, Homo sapiens
GLAVSTISSFFPPGYQ

SEQ ID NO: 4 - protein, 45 aa, Homo sapiens
TTTPAPRPPTPAPTIASQPLSLRPEACRPAAGGAVHTRGLDFACD

SEQ ID NO: 5 - protein, 231 aa, Homo sapiens
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, Homo sapiens
IYIWAPLAGTCGVLLLSLVITLYC

SEQ ID NO: 7 - protein, 27 aa, Homo sapiens
IISFFLALTSTALLFLLFFLTLRFSVV

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, Homo sapiens
GGGGSGGGGSGGGGS

SEQ ID NO: 11 - protein, 113 aa, artificial
  KLON43 VH
MADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKR

SEQ ID NO: 12 - protein, 127 aa, artificial
  KLON43 VL
EVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSS

SEQ ID NO: 13 - protein, 8 aa, artificial
  KLON 43 CDR1
GFTFTDYY

SEQ ID NO: 14 - protein, 9 aa, artificial
  KLON 43 CDR2
RSKADGYTT

SEQ ID NO: 15 - protein, 18 aa, artificial
  KLON 43 CDR3
ARDAAYYSYYSPEGAMDY

SEQ ID NO: 16 - protein, 6 aa, artificial
  KLON 43 CDR4
QNVDSA

SEQ ID NO: 17 - protein, 3 aa, artificial
  KLON 43 CDR5
SAS

SEQ ID NO: 18 - protein, 9 aa, artificial
  KLON 43 CDR6
QQYYSTPWT

SEQ ID NO: 19 - protein, 499 aa, artificial
  KLON43-CAR
MALPVTALLLPLALLLHAARPEVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSSGGGGSGGGGSGGGGSMADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 113 aa, artificial
  Humanized scFv Klon43 Variant VL1
MADYKDIVMTQSPSSVSASVGDRVTITCRASQNVDSAVAWYQQKPGKAPKALIYSASYRYSGVPSRFSGRGSGTDFTLTISSLQPEDFATYYCQQYYSTPWTFGQGTKVEIKR

SEQ ID NO: 21 - protein, 113 aa, artificial
  Humanized scFv Klon43 Variant VL2
MADYKDIQMTQSPSSVSASVGDRVTITCRASQNVDSAVAWYQQKPGKAPKALIYSASYRYSGVPSRFSGRGSGTDFTLTISSLQPEDFATYYCQQYYSTPWTFGQGTKVEIKR

SEQ ID NO: 22 - protein, 113 aa, artificial
  Humanized scFv Klon43 Variant VL3
MADYKDIQMTQSPSSVSASVGDRVTITCRASQNVDSAVAWYQQKPGKAPKALIYSASYRYSGVPSRFSGSGSGTDFTLTISSLQPEDFATYYCQQYYSTPWTFGQGTKVEIKR

SEQ ID NO: 23 - protein, 113 aa, artificial
  Humanized scFv Klon43 Variant VL4
MADYKDIQMTQSPSSVSASVGDRVTITCRASQNVDSAVAWYQQKPGKAPKLLIYSASYRYSGVPSRFSGSGSGTDFTLTISSLQPEDFATYYCQQYYSTPWTFGQGTKVEIKR

SEQ ID NO: 24 - protein, 113 aa, artificial
  Humanized scFv Klon43 Variant VL5
MADYKDIQMTQSPSSVSASVGDRVTITCRASQNVDSAVAWYQQKPGKAPKLLIYSASYRQSGVPSRFSGSGSGTDFTLTISSLQPEDFATYYCQQYYSTPWTFGQGTKVEIKR

SEQ ID NO: 25 - protein, 113 aa, artificial
  Humanized scFv Klon43 Variant VL6
MADYKDIQMTQSPSSVSASVGDRVTITCRASQNVDSAVAWYQQKPGKAPKLLIYSASYGQSGVPSRFSGSGSGTDFTLTISSLQPEDFATYYCQQYYSTPWTFGQGTKVEIKR

SEQ ID NO: 26 - protein, 127 aa, artificial
  Humanized scFv Klon43 Variant VH1
EVKLVESGGGLVQPGRSLRLSCTASGFTFTDYYMSWVRQAPGKGLEWVGLIRSKADGYTTEYSASVKGRFTISRDDSKSILYLQMNSLKTEDTAVYYCARDAAYYSYYSPEGAMDYWGQGTLVTVSS

SEQ ID NO: 27 - protein, 127 aa, artificial
  Humanized scFv Klon43 Variant VH2
EVQLVESGGGLVQPGRSLRLSCTASGFTFTDYYMSWVRQAPGKGLEWVGLIRSKADGYTTEYSASVKGRFTISRDDSKSILYLQMNSLKTEDTAVYYCARDAAYYSYYSPEGAMDYWGQGTLVTVSS

SEQ ID NO: 28 - protein, 127 aa, artificial
  Humanized scFv Klon43 Variant VH3
EVQLVESGGGLVQPGRSLRLSCTASGFTFTDYYMSWVRQAPGKGLEWVGLIRSKADGYTTEYSASVKGRFTISRDDSKSIAYLQMNSLKTEDTAVYYCARDAAYYSYYSPEGAMDYWGQGTLVTVSS

SEQ ID NO: 29 - protein, 127 aa, artificial
  Humanized scFv Klon43 Variant VH4
EVQLVESGGGLVQPGRSLRLSCTASGFTFTDYYMSWVRQAPGKGLEWVGFIRSKADGYTTEYSASVKGRFTISRDDSKSIAYLQMNSLKTEDTAVYYCARDAAYYSYYSPEGAMDYWGQGTLVTVSS

SEQ ID NO: 30 - protein, 127 aa, artificial
  Humanized scFv Klon43 Variant VH5
EVQLVESGGGLVQPGRSLRLSCTASGFTFTDYYMSWVRQAPGKGLEWVGFIRSKADGYTTEYAASVKGRFTISRDDSKSIAYLQMNSLKTEDTAVYYCARDAAYYSYYSPEGAMDYWGQGTLVTVSS

SEQ ID NO: 31 - protein, 127 aa, artificial
  Humanized scFv Klon43 Variant VH6
EVQLVESGGGLVQPGRSLRLSCTASGFTFTDYYMSWVRQAPGKGLEWVGLIRSKADGYTTEYAASVKGRFTISRDDSKSIAYLQMNSLKTEDTAVYYCARDAAYYSYYSPEGAMDYWGQGTLVTVSS

SEQ ID NO: 32 - protein, 127 aa, artificial
  Humanized scFv Klon43 Variant VH7
EVQLVESGGGLVQPGRSLRLSCTASGFTFTDYYMSWVRQAPGKGLEWVGFIRSKADGYTTEYAASVKGRFTISRDDSKSIAYLQMNSLKTEDTAVYYCTRDAAYYSYYSPEGAMDYWGQGTLVTVSS

SEQ ID NO: 33 - protein, 120 aa, artificial
  CLL1 VH
EVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSS

SEQ ID NO: 34 - protein, 108 aa, artificial
  CLL1 VL
DIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKR

SEQ ID NO: 35 - protein, 526 aa, artificial
  CLL1-CAR-R2
MALPVTALLLPLALLLHAARPEVQLQQSGPELVKPGASVKMSCKASGYTFTSYFIHWVKQKPGQGLEWIGFINPYNDGSKYNEKFKGKATLTSDKSSSTAYMELSSLTSEDSAVYYCTRDDGYYGYAMDYWGQGTSVTVSSGGGGSGGGGSGGGGSDIQMTQSPSSLSASLGERVSLTCRATQELSGYLSWLQQKPDGTIKRLIYAASTLDSGVPKRFSGNRSGSDYSLTISSLESEDFADYYCLQYAIYPYTFGGGTKLEIKSDPGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 500 aa, Homo sapiens
MSRSVALAVLALLSLSGLEAVMAPRTLILGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 37 - protein, 500 aa, Homo sapiens
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 38 - protein, 734 aa, Homo sapiens
MSRSVALAVLALLSLSGLEAVMAPRTLFLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSLGSGVKQTLNFDLLKLAGDVESNPGPMVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEADPPKTHVTHHPVFDYEATLRCWALGFYPAEIILTWQRDGEDQTQDVELVETRPAGDGTFQKWAAVVVPSGEEQRYTCHVQHEGLPEPLMLRWKQG

SEQ ID NO: 39 - protein, 554 aa, Homo sapiens
MERRRGTVPLGWVFFVLCLSASSSCAVDLGSKSSNSTCRLNVTELASIHPGETWTLHGMCISICYYENVTEDEIIGVAFTWQHNESVVDLWLYQNDTVIRNFSDITTNILQDGLKMRTVPVTKLYTSRMVTNLTVGRYDCLRCENGTTKIIERLYVRLGSLYPRPPGSGLAKHPSVSADEELSATLARDIVLVSAITLFFFLLALRIPQRLCQRLRIRLPHRYQRLRTEDEGRGSLLTCGDVEENPGPMRIEWVWWLFGYFVSSVGSERSLSYRYHLESNSSTNVVCNGNISVFVNGTLGVRYNITVGISSSLLIGHLTIQVLESWFTPWVQNKSYNKQPLGDTETLYNIDSENIHRVSQYFHTRWIKSLQENHTCDLTNSTPTYTYQVNVNNTNYLTLTSSGWQDRLNYTVINSTHFNLTESNITSIQKYLNTTCIERLRNYTLESVYTTTVPQNITTSQHATTTMHTIPPNTITIQNTTQSHTVQTPSFNDTHNVTKHTLNISYVLSQKTNNTTSPWIYAIPMGATATIGAGLYIGKHFTPVKFVYEVWRGQ

SEQ ID NO: 40 - protein, 925 aa, artificial
  TRAC_T01-L TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 41 - protein, 925 aa, artificial
  TRAC_T01-R TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 42 - protein, 925 aa, artificial
  CD52_T01-L TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 43 - protein, 925 aa, artificial
  CD52_T01-R TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 44 - protein, 925 aa, artificial
  B2M_T02-L4 TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 45 - protein, 925 aa, artificial
  B2M_T02-R TALEN
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 46 - DNA, 49 bp, Homo sapiens
ttgtcccacagatatccagaaccctgaccctgccgtgtaccagctgaga

SEQ ID NO: 47 - DNA, 49 bp, Homo sapiens
ttcctcctactcaccatcagcctcctggttatggtacaggtaagagcaa

SEQ ID NO: 48 - DNA, 47 bp, Homo sapiens
ttagctgtgctcgcgctactctctctttctggcctggaggctatcca